(19) United States
(12) Patent Application Publication
(10) Pub. No.: US 2024/0392035 A1
(43) Pub. Date: Nov. 28, 2024

(54) ENGINEERED ANTI-HER2 BISPECIFIC PROTEINS

(71) Applicant: DENALI THERAPEUTICS INC., South San Francisco, CA (US)

(72) Inventors: Abira BANDYOPADHYAY, Pleasanton, CA (US); Allisa Jayne CLEMENS, San Jose, CA (US); Do Jin KIM, Albany, CA (US); Michelle E. PIZZO, Pacifica, CA (US); Lu SHAN, Belmont, CA (US); Richard THÉOLIS, Jr., Santa Cruz, CA (US); Raymond Ka Hang TONG, South San Francisco, CA (US)

(21) Appl. No.: 18/685,583

(22) PCT Filed: Aug. 25, 2022
(86) PCT No.: PCT/US2022/041471

§ 371 Date: Feb. 22, 2024

Related U.S. Application Data
(60) Provisional application No. 63/237,071, filed on Aug. 25, 2021.

(51) Int. Cl. C07K 16/32; A61K 39/00; A61P 35/00

(52) U.S. Cl. CPC C07K 16/32; A61P 35/00; A61K 2039/505; C07K 2317/31; C07K 2317/52; C07K 2317/55; C07K 2317/622; C07K 2317/71

(57) ABSTRACT

In one aspect, bispecific proteins having the ability to specifically bind to both subdomain II of human HER2 and subdomain IV of human HER2 are provided. In another aspect, methods of treating a cancer or treating brain metastasis of a cancer using a bispecific protein that specifically binds to subdomain II and subdomain IV of human HER2 are provided.

### CROSS-REFERENCE TO RELATED APPLICATIONS

The present application claims priority to U.S. Provisional Patent Application No. 63/237,071, filed on Aug. 25, 2021, the disclosure of which is incorporated herein by reference in its entirety for all purposes.

### BACKGROUND

Treatment of brain metastases of cancers such as breast cancer currently poses a daunting clinical challenge. Among breast cancer patients, the incidence of brain metastases is as high as 50%. Clinical data indicate that there is a proclivity for HER2-positive breast cancers to metastasize to the brain. Notably, anti-HER2 therapies have proven useful for the control of extracranial tumors but not intracranial lesions. The failure of these therapies to control metastatic lesions such as brain metastases of HER2-positive breast cancer is mostly attributed to an inability of the therapeutic agents to cross the blood brain barrier (BBB) and access the brain parenchyma.

### SUMMARY

In one aspect, the disclosure provides a protein comprising:
(a) a first Fc polypeptide that is fused at the N-terminus to an Fd portion of a Fab;
(b) a second Fc polypeptide that is fused at the N-terminus to a single-chain variable fragment (scFv), wherein the first and second Fc polypeptides form an Fc dimer; and
(c) a light chain polypeptide that pairs with the Fd portion recited in (a) to form a Fab,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.

In some embodiments of this protein, the first Fc polypeptide and/or the second Fc polypeptide independently comprising the S239D and/or the I332E substitution is capable of enhancing HER2-mediated effector function.

In some embodiments of this protein, the first Fc polypeptide or the second Fc polypeptide comprises a S239D and/or a I332E substitution, according to EU numbering. In other embodiments, the first Fc polypeptide comprises a S239D and/or a I332E substitution and the second Fc polypeptide comprises a S239D and/or a I332E substitution, according to EU numbering.

In some embodiments of this protein,
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.

In certain embodiments of this protein,
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.

In particular embodiments of this protein,
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at 332.

In some embodiments of this protein, the Fab binds to subdomain II of human HER2 and the scFv binds to subdomain IV of human HER2. In other embodiments, the Fab binds to subdomain IV of human HER2 and the scFv binds to subdomain II of human HER2.

In some embodiments of this protein, the second Fc polypeptide is fused to the scFv via a first linker. The first linker can have a length from 1 to 20 amino acids, e.g., a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments of this protein, the scFv comprises a V_{L} region and a V_{H} region that are connected via a second linker. The second linker can have a length from 1 to 20 amino acids, e.g., a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments of this protein, the first Fc polypeptide and/or the second Fc polypeptide specifically binds to a transferrin receptor (TfR), e.g., contains any of the sequence modifications described herein that create a TfR-binding site. In some embodiments, the first Fc polypeptide and the second Fc polypeptide each comprises modifications that promote heterodimerization. In certain embodiments, the first Fc polypeptide comprises a T366W substitution and the second Fc polypeptide comprises T366S, L368A, and Y407V substitutions, according to EU numbering. In other embodiments, the first Fc polypeptide comprises T366S, L368A, and Y407V substitutions and the second Fc polypeptide comprises a T366W substitution, according to EU numbering.

In some embodiments of this protein, the first Fc polypeptide and/or the second Fc polypeptide independently comprises modifications that reduce TfR-mediated effector function. In certain embodiments, the modifications that reduce effector function are L234A and L235A substitutions, according to EU numbering. As one example, the first Fc polypeptide may specifically bind to TfR and comprise L234A and L235A substitutions, the first Fc polypeptide may further comprise a P329G or a P329S substitution, and the second Fc polypeptide may comprise Leu at positions 234 and 235 and a proline at position 329, according to EU numbering. As another example, the second Fc polypeptide may specifically bind to TfR and comprise L234A and L235A substitutions, the second Fc polypeptide may further comprise a P329G or a P329S substitution, and the first Fc polypeptide may comprise Leu at positions 234 and 235 and a proline at position 329, according to EU numbering.

In some embodiments of this protein, a hinge region or a portion thereof is linked to the N-terminus of the first Fc polypeptide and/or the second Fc polypeptide.

In some embodiments of this protein, the first Fc polypeptide and/or the second Fc polypeptide independently comprises a sequence having at least 90% (e.g., 91%, 92%, 93%, 94%, 95%, 96%, 97%, 98%, 99%, or 100%) identity to a sequence selected from the group consisting of SEQ ID NOS:131-149 and 183-196. In certain embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from SEQ ID NOS:133 and 183-185. In certain other embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from SEQ ID NOS:137 and 186-196.

In some embodiments of this protein, the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:135-139 and 186-196.

In some embodiments of this protein, the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to the sequence of SEQ ID NO:137, and the second Fc polypeptide comprises Ser at position 366, Ala at position 368, and Val at position 407, according to EU numbering, and a sequence having at least 90% (e.g., 91%, 92%, 93%, 94%, 95%, 96%, 97%, 98%, 99%, or 100%) identity to the sequence of SEQ ID NO:133.

In other embodiments of this protein, the first Fc polypeptide comprises Ser at position 366, Ala at position 368, and Val at position 407, according to EU numbering, and a sequence having at least 90% (e.g., 91%, 92%, 93%, 94%, 95%, 96%, 97%, 98%, 99%, or 100%) identity to the sequence of SEQ ID NO:133, and the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to the sequence of SEQ ID NO:137.

In another aspect, the disclosure provides a protein comprising:
(a) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:1, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:159, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(b) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:164, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:173, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(c) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:164, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:174, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(d) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:166, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:173, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(e) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:165, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:173, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(f) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:165, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:174, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(g) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:167, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:173, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(h) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:164, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:175, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(i) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:164, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:176, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(j) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:166, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:175, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(k) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:165, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:175, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(l) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:165, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:176, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25; or
(m) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:167, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:175, and (iii) a light chain polypeptide comprising a sequence having at least 90% (e.g., 91%, 92%, 93%, 94%, 95%, 96%, 97%, 98%, 99%, or 100%) identity to the sequence of SEQ ID NO:25.

In some embodiments of this protein, the first heavy chain polypeptide comprises a TfR-binding site, modifications that promote heterodimerization, modifications that enhance HER2-mediated effector function, and/or modifications that reduce TfR-mediated effector function present in a first heavy chain polypeptide sequence, and a sequence having at least 90% (e.g., 91%, 92%, 93%, 94%, 95%, 96%, 97%, 98%, 99%, or 100%) identity to the first heavy chain polypeptide sequence. In other embodiments of this protein, the second heavy chain polypeptide comprises a TfR-binding site, modifications that promote heterodimerization, modifications that enhance HER2-mediated effector function, and/or modifications that reduce TfR-mediated effector function present in a second heavy chain polypeptide sequence, and a sequence having at least 90% (e.g., 91%, 92%, 93%, 94%, 95%, 96%, 97%, 98%, 99%, or 100%) identity to the second heavy chain polypeptide sequence.

In yet another aspect, the disclosure provides a protein comprising:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab,
wherein the Fd portion in (a) and/or (b) is fused at the N-terminus to an scFv,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.

In some embodiments of this protein, the first Fc polypeptide and/or the second Fc polypeptide independently comprising the S239D and/or the I332E substitution is capable of enhancing HER2-mediated effector function.

In some embodiments of this protein, the first Fc polypeptide or the second Fc polypeptide comprises a S239D and/or a I332E substitution, according to EU numbering. In other embodiments, the first Fc polypeptide comprises a S239D and/or a I332E substitution and the second Fc polypeptide comprises a S239D and/or a I332E substitution, according to EU numbering.

In some embodiments of this protein,
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.

In certain embodiments of this protein,
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.

In particular embodiments of this protein,
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at 332.

In some embodiments of this protein, the Fab binds to subdomain II of human HER2 and the scFv binds to subdomain IV of human HER2. In other embodiments, the Fab binds to subdomain IV of human HER2 and the scFv binds to subdomain II of human HER2.

In some embodiments of this protein, the Fd portion in (a) or (b) is fused at the N-terminus to the scFv.

In some embodiments of this protein, the Fd portion in (a) and/or (b) is fused to the scFv via a first linker. In certain embodiments, the first linker has a length from 1 to 20 amino acids, e.g., a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments of this protein, the scFv comprises a V_{L} region and a V_{H} region that are connected via a second linker. In some embodiments, the second linker has a length from 1 to 20 amino acids, e.g., a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO: 123).

In some embodiments of this protein, the first Fc polypeptide and/or the second Fc polypeptide specifically binds to a transferrin receptor (TfR), e.g., contains any of the sequence modifications described herein that create a TfR-binding site. In some embodiments, the first Fc polypeptide and the second Fc polypeptide each comprises modifications that promote heterodimerization. In certain embodiments, the first Fc polypeptide comprises a T366W substitution and the second Fc polypeptide comprises T366S, L368A, and Y407V substitutions, according to EU numbering. In other embodiments, the first Fc polypeptide comprises T366S, L368A, and Y407V substitutions and the second Fc polypeptide comprises a T366W substitution, according to EU numbering.

In some embodiments of this protein, the first Fc polypeptide and/or the second Fc polypeptide independently comprises modifications that reduce TfR-mediated effector function. In certain embodiments, the modifications that reduce effector function are L234A and L235A substitutions, according to EU numbering. As one example, the first Fc polypeptide may specifically bind to TfR and comprise L234A and L235A substitutions, the first Fc polypeptide may further comprise a P329G or a P329S substitution, and the second Fc polypeptide may comprise Leu at positions 234 and 235 and a proline at position 329, according to EU numbering. As another example, the second Fc polypeptide may specifically bind to TfR and comprise L234A and L235A substitutions, the second Fc polypeptide may further comprise a P329G or a P329S substitution, and the first Fc polypeptide may comprise Leu at positions 234 and 235 and a proline at position 329, according to EU numbering.

In some embodiments of this protein, a hinge region or a portion thereof is linked to the N-terminus of the first Fc polypeptide and/or the second Fc polypeptide.

In some embodiments of this protein, the first Fc polypeptide and/or the second Fc polypeptide independently comprises a sequence having at least 90% (e.g., 91%, 92%, 93%, 94%, 95%, 96%, 97%, 98%, 99%, or 100%) identity to a sequence selected from the group consisting of SEQ ID NOS:131-149 and 183-196. In certain embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from SEQ ID NOS:133 and 183-185. In certain other embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from SEQ ID NOS:137 and 186-196.

In some embodiments of this protein, the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:135-139 and 186-196.

In some embodiments of this protein, the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to the sequence of SEQ ID NO:137, and the second Fc polypeptide comprises Ser at position 366, Ala at position 368, and Val at position 407, according to EU numbering, and a sequence having at least 90% (e.g., 91%, 92%, 93%, 94%, 95%, 96%, 97%, 98%, 99%, or 100%) identity to the sequence of SEQ ID NO:133.

In other embodiments of this protein, the first Fc polypeptide comprises Ser at position 366, Ala at position 368, and Val at position 407, according to EU numbering, and a sequence having at least 90% (e.g., 91%, 92%, 93%, 94%, 95%, 96%, 97%, 98%, 99%, or 100%) identity to the sequence of SEQ ID NO:133, and the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to the sequence of SEQ ID NO:137.

In a further aspect, the disclosure provides a protein comprising:
(a) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:6, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:160, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(b) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:6, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:161, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(c) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:6, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:162, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(d) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:177, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(e) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:178, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(f) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:171, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:177, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(g) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO: 170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:177, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(h) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:178, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(i) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:172, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:177, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(j) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:179, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(k) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:180, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(l) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:171, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:179, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(m) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:179, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(n) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:180, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(o) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:172, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:179, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(p) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:181, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(q) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:169, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:182, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(r) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:171, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:181, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(s) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:181, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(t) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:170, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:182, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26; or
(u) (i) a first heavy chain polypeptide comprising a sequence having at least 90% (e.g., 91%, 92%, 93%, 94%, 95%, 96%, 97%, 98%, 99%, or 100%) identity to the sequence of SEQ ID NO:172, (ii) a second heavy chain polypeptide comprising a sequence having at least 90% (e.g., 91%, 92%, 93%, 94%, 95%, 96%, 97%, 98%, 99%, or 100%) identity to the sequence of SEQ ID NO:181, and (iii) two light chain polypeptides each comprising a sequence having at least 90% (e.g., 91%, 92%, 93%, 94%, 95%, 96%, 97%, 98%, 99%, or 100%) identity to the sequence of SEQ ID NO:26.

In some embodiments of this protein, the first heavy chain polypeptide comprises a TfR-binding site, modifications that promote heterodimerization, modifications that enhance HER2-mediated effector function, and/or modifications that reduce TfR-mediated effector function present in a first heavy chain polypeptide sequence, and a sequence having at least 90% (e.g., 91%, 92%, 93%, 94%, 95%, 96%, 97%, 98%, 99%, or 100%) identity to the first heavy chain polypeptide sequence. In other embodiments of this protein, the second heavy chain polypeptide comprises a TfR-binding site, modifications that promote heterodimerization, modifications that enhance HER2-mediated effector function, and/or modifications that reduce TfR-mediated effector function present in a second heavy chain polypeptide sequence, and a sequence having at least 90% (e.g., 91%, 92%, 93%, 94%, 95%, 96%, 97%, 98%, 99%, or 100%) identity to the second heavy chain polypeptide sequence.

In another aspect of the disclosure, the disclosure provides a pharmaceutical composition comprising any of the proteins described herein and a pharmaceutically acceptable carrier.

In another aspect of the disclosure, the disclosure provides an isolated polynucleotide comprising a nucleotide sequence encoding a protein described herein.

In another aspect of the disclosure, the disclosure provides a vector comprising the polynucleotide of the previous aspect.

In another aspect of the disclosure, the disclosure provides a host cell comprising the polynucleotide or the vector.

In another aspect of the disclosure, the disclosure provides a method for treating a cancer or treating brain metastasis of a cancer in a subject, the method comprising administering to the subject a therapeutically effective amount of a protein described herein or a pharmaceutical composition thereof.

In some embodiments of the method, the protein is adminstered in combination with a chemotherapy or radiation therapy.

In some embodiments of the method, the cancer is a metastatic cancer. In some embodiments, the cancer is a breast cancer. In some embodiments, the cancer is a HER2-positive cancer.

### BRIEF DESCRIPTION OF THE DRAWINGS

FIG. 1A is a schematic drawing showing an exemplary bispecific protein having the “Fab-Fc polypeptide/scFv-Fc polypeptide” structure, in which the scFv is fused to the N-terminus of an Fc polypeptide having a TfR-binding site (starred) and a knob mutation via a hinge or a partial hinge region, while the other Fc polypeptide has a hole mutation.

FIG. 1B is a schematic drawing showing an exemplary bispecific protein having the “Fab-Fc polypeptide/scFv-Fc polypeptide” structure, in which the scFv is fused to the N-terminus of an Fc polypeptide a hole mutation via a hinge or a partial hinge region, while the other Fc polypeptide has a TfR-binding site (starred) and a knob mutation.

FIG. 2A is a schematic drawing showing an exemplary bispecific protein having the “mAb/N-terminal or C-terminal scFv on HC” structure, in which the scFv is fused to the C-terminus of an Fc polypeptide having a hole mutation, while the other Fc polypeptide has a TfR-binding site (starred) and a knob mutation.

FIG. 2B is a schematic drawing showing an exemplary bispecific protein having the “mAb/N-terminal or C-terminal scFv on HC” structure, in which the scFv is fused to the N-terminus of an Fd portion via a linker. The scFv is fused to the Fd portion of the heavy chain containing an Fc polypeptide having a hole mutation, while the other Fc polypeptide has a TfR-binding site (starred) and a knob mutation.

FIG. 2C is a schematic drawing showing an exemplary bispecific protein having the “mAb/N-terminal or C-terminal scFv on HC” structure, in which two scFvs are each fused to the N-terminus of the Fd portion of a heavy chain via a linker. The TfR-binding site on the Fc polypeptide is denoted by star.

FIG. 2D is a schematic drawing showing an exemplary bispecific protein having the “mAb/N-terminal or C-terminal scFv on HC” structure, in which two scFvs are each fused to the C-terminus of the Fc polypeptide of a heavy chain via a linker. The TfR-binding site on the Fc polypeptide is denoted by star.

FIG. 3A is a schematic drawing showing an exemplary bispecific protein having the “mAb/N-terminal or C-terminal scFv on LC” structure, in which the scFv is fused to the C-terminus of a light chain via a linker. The TfR-binding site on the Fc polypeptide is denoted by star.

FIG. 3B is a schematic drawing showing an exemplary bispecific protein having the “mAb/N-terminal or C-terminal scFv on LC” structure, in which the scFv is fused to the N-terminus of a light chain via a linker. The TfR-binding site on the Fc polypeptide is denoted by star.

FIG. 3C is a schematic drawing showing an exemplary bispecific protein having the “mAb/N-terminal or C-terminal scFv on LC” structure, in which two scFvs are each fused to the N-terminus of a light chain via a linker. The TfR-binding site on the Fc polypeptide is denoted by star.

FIG. 3D is a schematic drawing showing an exemplary bispecific protein having the “mAb/N-terminal or C-terminal scFv on LC” structure, in which two scFvs are each fused to the C-terminus of a light chain via a linker. The TfR-binding site on the Fc polypeptide is denoted by star.

FIG. 4 is a schematic drawing showing an exemplary bispecific protein having the “mAb/N-terminal V_{H} V_{L} on HC and LC” structure, in which two V_{H} regions are each fused to the N-terminus of the Fd portion of a heavy chain and two V_{L} regions are each fused to the N-terminus of a light chain. A V_{H} region and a V_{L} region form an Fv fragment. The TfR-binding site on the Fc polypeptide is denoted by star.

FIG. 5 is a schematic drawing showing an exemplary bispecific protein having the “mAb/C-terminal V_{H} V_{L} on HC” structure, in which a V_{H} region is fused to the C-terminus of an Fc polypeptide having a hole mutation and a V_{L} region is fused to the C-terminus of an Fc polypeptide having a TfR-binding site (starred) and a knob mutation. A V_{H} region and a V_{L} region form an Fv fragment.

FIGS. 6A and 6B show the inhibition of cancer cell proliferation by bispecific proteins having the “Fab-Fc polypeptide/scFv-Fc polypeptide” structure on Day 6 and Day 3 in a growth inhibition assay of BT474 and OE19 cells, respectively.

FIG. 7 is a plot showing the plasma PK profile of anti-HER2 bispecific proteins and anti-HER2 controls in C57/BL6 mice.

FIG. 8A is a plot showing the reticulocyte quantification in TfR^{mu/hu} KI mice intravenously administered anti-HER2 bispecific proteins or anti-HER2 controls.

FIG. 8B is a plot showing the plasma PK profile of anti-HER2 bispecific proteins and anti-HER2 controls in TfR^{mu/hu} KI mice.

FIG. 8C is a plot showing the brain PK profile of anti-HER2 bispecific proteins and anti-HER2 controls in TfR^{mu/hu} KI mice.

FIG. 8D is a plot showing the percentage of brain-to-plasma concentrations of anti-HER2 bispecific proteins and anti-HER2 controls in TfR^{mu/hu} KI mice.

FIGS. 9A-9F show growth inhibition assays of BT474 cells with (FIGS. 9A-9C) or without (FIGS. 9D-9F) NRG1 by anti-HER2 bispecific proteins and anti-HER2 controls.

FIGS. 9G-9I show a growth inhibition assay of OE19 cells by anti-HER2 bispecific proteins and anti-HER2 controls.

FIGS. 9J-9L show a growth inhibition assay of ZR75 cells by anti-HER2 bispecific proteins and anti-HER2 controls.

### DETAILED DESCRIPTION

### I. Introduction

In one aspect, bispecific proteins that can bind to both subdomain II of human HER2 and subdomain IV of human HER2 are provided. The bispecific proteins can, in general, be generated without light chain mispairing or steering. In some embodiments, the bispecific proteins bind to each target subdomain of human HER2 monovalently. In some embodiments, the bispecific proteins bind to one target subdomain of human HER2 monovalently and the other target subdomain of human HER2 bivalently (e.g., to subdomain II monovalently and to subdomain IV bivalently, or to subdomain IV monovalently and to subdomain II bivalently). In some embodiments, the bispecific proteins bind to each target subdomain of human HER2 bivalently. Various structures of the bispecific proteins are described in detail further herein.

In some embodiments, the bispecific protein comprises an scFv that binds to subdomain II (or subdomain IV) of human HER2 and a Fab that binds to subdomain IV (or subdomain II) of human HER2 (see, e.g., “Fab-Fc polypeptide/scFv-Fc polypeptide” structure in Section III). In some embodiments, the bispecific protein comprises one or more scFvs that are connected to the N- or C-terminus of the heavy chains of the bispecific protein, in which the scFv binds to subdomain II (or subdomain IV) of human HER2 and the Fab in the bispecific protein binds to subdomain IV (or subdomain II) of human HER2 (see, e.g., “mAb/N-terminal or C-terminal scFv on HUC” structure in Section III). In some embodiments, the bispecific protein comprises one or more scFvs that are connected to the N- or C-terminus of the light chains of the bispecific protein, in which the scFv binds to subdomain II (or subdomain IV) of human HER2 and the Fab in the bispecific protein binds to subdomain IV (or subdomain II) of human HER2 (see, e.g., “mAb/N-terminal or C-terminal scFv on HC mAb/N-terminal or C-terminal scFv on LC” structure in Section III). In some embodiments, the bispecific protein comprises a V_{H} region (or a V_{L} region) of an Fv fragment connected to the N-terminus of the heavy chains and a V_{L} region (or a V_{H} region) of the Fv fragment connected to the N-terminus of the light chains, in which the Fv fragment binds to subdomain II (or subdomain IV) of human HER2 and the Fab in the bispecific protein binds to subdomain IV (or subdomain II) of human HER2 (see, e.g., “mAb/N-terminal V_{H} V_{L} on HC and LC” structure in Section III). In yet other embodiments, the bispecific protein comprises a V_{H} region (or a V_{L} region) of an Fv fragment connected to the C-terminus of one of the two heavy chains in the bispecific protein and a V_{L} region (or a V_{H} region) of the Fv fragment connected to the C-terminus of the other of the two heavy chains, in which the Fv fragment binds to subdomain II (or subdomain IV) of human HER2 and the Fab in the bispecific protein binds to subdomain IV (or subdomain II) of human HER2 (see, e.g., “mAb/C-terminal V_{H} V_{L} on HUC” structure in Section III).

Previous therapies have failed to control brain metastases of HER2-positive breast cancer mostly because of the inability of the therapeutic agents to cross the blood brain barrier (BBB) and access the brain parenchyma. Thus, there is a need for new therapeutic agents that can cross the BBB and target HER2 in the brain parenchyma. We previously described the use of transferrin receptor (TfR)-binding as a method to enable BBB delivery across the brain endothelium, as the expression of TfR is highly expressed in brain endothelial cells and can enable BBB delivery via receptor-mediated transcytosis. Interestingly, TfR is highly expressed in various cancers, including HER2-positive breast cancers. The mechanism by which cancer cells acquire increased TfR expression likely relates to tumor cell proliferation and increased metabolic demand such as iron uptake. In fact, public microarray datasets demonstrated a correlation of TfR expression to breast cancer prognosis (Miller et al., Cancer Res. 71:6728, 2011). There have also been some reports on the use of TfR as a pharmacological target for various types of cancers.

In some embodiments, the bispecific protein comprises one or more modified Fc polypeptides that specifically bind to a BBB receptor, e.g., TfR (i.e., TfR-binding Fc polypeptides). In some embodiments, the bispecific protein is capable of being transported across the BBB. In some embodiments, the anti-HER2 bispecific proteins binding to both HER2 and TfR as described herein can provide additional anti-tumor benefits upon binding to HER2-positive tumor cells which also express high levels of TfR, compared to other therapeutic agents that bind to HER2 alone. Specifically, since these proteins can bind both the TfR and HER2 at the same time, this could enhance their potency and/or efficacy.

In some embodiments, the bispecific protein comprises a modified Fc polypeptide dimer that specifically binds TfR, has reduced effector function (e.g., ADCC or CDC) when bound to TfR, but retains or has enhanced effector function (e.g., ADCC or CDC) when bound to HER2.

### II. Definitions

As used herein, the singular forms “a,” “an,” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain antigen-binding specificity, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (C and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 or CDR-H3 is located in the variable region of the heavy chain of the antibody in which it is found, whereas a V_{L} CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol. 262:732-745, 1996. In some embodiments, CDRs are determined by a combination of Kabat, Chothia, and/or Contact CDR definitions.

The term “Fd portion” refers to an N-terminal portion of an immunoglobulin heavy chain. Typically, an Fd portion includes the heavy chain variable (V_{H}) region and a heavy chain constant (CH1) region.

The term “Fab” refers to an antigen-binding fragment consisting of a light chain variable region, a light chain constant region, a heavy chain variable region, and a heavy chain CH1 constant region.

The term “single-chain variable fragment” or “scFv” refers to an antigen-binding fragment consisting of a heavy chain variable region and a light chain variable region linked together via a peptide linker. An scFv lacks constant regions.

The term “Fv fragment” refers to an antigen-binding fragment consisting of a heavy chain variable region and a light chain variable region that together form a binding site for an antigen.

The term “epitope” refers to the area or region of an antigen to which a molecule, e.g., the CDRs of an antibody, specifically binds and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids (e.g., a linear epitope), or amino acids from different parts of the protein that are brought into proximity by protein folding (e.g., a discontinuous or conformational epitope).

As used herein, the phrase “recognizes an epitope,” as used with reference to an antibody, means that the antibody CDRs interact with or specifically bind to the antigen at that epitope or a portion of the antigen containing that epitope.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) variable domain(s), in which the CDR regions substantially correspond to those of the non-human immunoglobulin and the framework regions substantially correspond to those of a human immunoglobulin sequence. In some instances, certain framework region residues of a human immunoglobulin can be replaced with the corresponding residues from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., a Fab, an scFv, or a modified Fc polypeptide (or a target-binding portion thereof) that binds to an epitope or target with greater affinity, greater avidity, and/or greater duration to that epitope or target in a sample than it binds to another epitope or non-target compound (e.g., a structurally different antigen). In some embodiments, a Fab, scFv, or modified Fc polypeptide (or a target-binding portion thereof) that specifically binds to an epitope or target is a Fab, scFv, or modified Fc polypeptide (or a target-binding portion thereof) that binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻¹ M, 10⁻⁹ M, 10⁻¹⁰ M, 10⁻¹¹ M, or 10⁻¹² M. It will be recognized by one of skill that a Fab or scFv that specifically binds to a target from one species may also specifically bind to orthologs of that target.

The term “binding affinity” is used herein to refer to the strength of a non-covalent interaction between two molecules, e.g., between a Fab or scFv and an antigen, or between a modified Fc polypeptide (or a target-binding portion thereof) and a target. Thus, for example, the term may refer to 1:1 interactions between a Fab or scFv and an antigen or between a modified Fc polypeptide (or a target-binding portion thereof) and a target, unless otherwise indicated or clear from context. Binding affinity may be quantified by measuring an equilibrium dissociation constant (K_{D}), which refers to the dissociation rate constant (k_{d}, time⁻¹) divided by the association rate constant (k_{d}, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between a Fab or scFv and an antigen or between a modified Fc polypeptide (or a target-binding portion thereof) and a target, but also apparent affinities for which K_{D}'s are calculated that may reflect avid binding.

A “transferrin receptor” or “TfR,” as used herein, refers to transferrin receptor protein 1. The human transferrin receptor 1 polypeptide sequence is set forth in SEQ ID NO:150. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes L251, M252, I253, S254, R255, T256, M428, H433, N434, H435, and Y436. These positions correspond to positions 21 to 26, 198, and 203 to 206 of SEQ ID NO:130.

As used herein, a “native FcRn binding site” refers to a region of an Fc polypeptide that binds to FcRn and that has the same amino acid sequence as the region of a naturally occurring Fc polypeptide that binds to FcRn.

As used herein, the terms “CH3 domain” and “CH2 domain” refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:127).

The terms “wild-type,” “native,” and “naturally occurring,” as used with reference to a CH3 or CH2 domain, refer to a domain that has a sequence that occurs in nature.

As used herein, the term “mutant,” as used with reference to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “isolated,” as used with reference to a nucleic acid or protein, denotes that the nucleic acid or protein is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring are those encoded by the genetic code, as well as those amino acids that are later modified, e.g., hydroxyproline, γ-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “linker,” as used herein, refers to a moiety that links (e.g., covalently links) two peptides or polypeptides (e.g., between an Fc polypeptide and an scFv) to connect or fuse the peptides or polypeptides. In some embodiments, a linker comprises a chemical linkage. In some embodiments, a linker comprises a peptide having a length of one or more amino acid residues. Suitable linkers for connecting or fusing peptides or polypeptides can be selected based on the properties of the linkers, such as the length, hydrophobicity, flexibility, rigidity, or cleavability of the linker.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:130, when the residue aligns with the amino acid in SEQ ID NO:130 when optimally aligned to SEQ ID NO:130. The polypeptide that is aligned to the reference sequence need not be the same length as the reference sequence.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent is an amount of the agent (e.g., any of the proteins described herein) that treats a disease in a subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, a protein as described herein is administered intravenously.

### III. Anti-HER2 Bispecific Proteins

In one aspect, bispecific proteins that have the ability to specifically bind to both subdomain II of human HER2 and subdomain IV of human HER2 are provided. In some embodiments, one or both of the Fc polypeptides of the bispecific protein is a modified Fc polypeptide (e.g., modified to promote TfR binding and/or to enhance heterodimerization of the Fc polypeptides).

### Fab-Fc Polypeptide/scFv-Fc Polypeptide

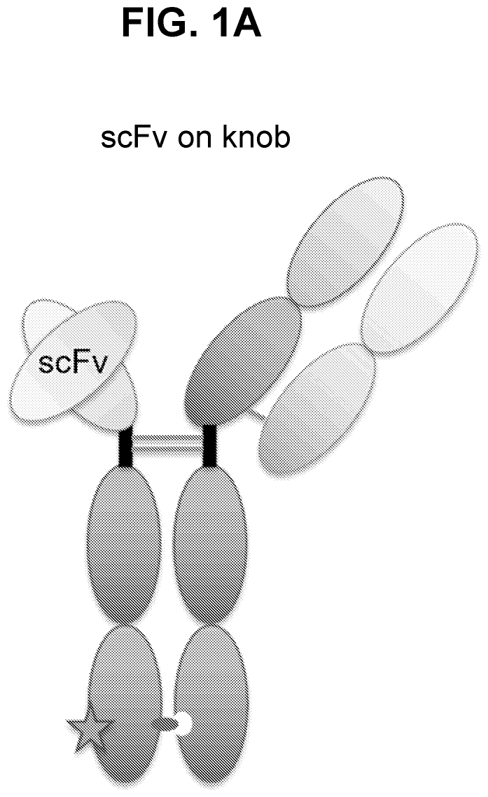

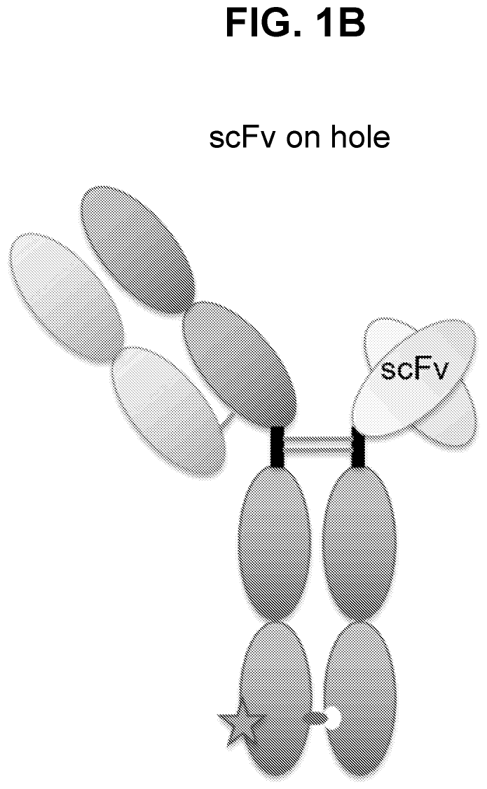

In some embodiments, a bispecific protein comprises Fc polypeptides that are fused to a portion of a Fab and an scFv. Schematic drawings of such a bispecific protein are shown in FIGS. 1A and 1B. In some embodiments, the bispecific protein comprises:
(a) a first Fc polypeptide that is fused at the N-terminus to an Fd portion of a Fab;
(b) a second Fc polypeptide that is fused at the N-terminus to a single-chain variable fragment (scFv), wherein the first and second Fc polypeptides form an Fc dimer; and
(c) a light chain polypeptide that pairs with the Fd portion recited in (a) to form a Fab,
wherein the Fab binds to subdomain II of human HER2 and the scFv binds to subdomain IV of human HER2, or wherein the Fab binds to subdomain IV of human HER2 and the scFv binds to subdomain II of human HER2. In some embodiments, the Fab in the protein binds to subdomain II of human HER2 and the scFv binds to subdomain IV of human HER2. In other embodiments, the Fab in the protein binds to subdomain IV of human HER2 and the scFv binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

The Fab is formed from the pairing of the Fd portion of the Fab, which is fused to the N-terminus of the first Fc polypeptide, with the light chain. In some embodiments, the Fab that specifically binds to the subdomain II of human HER2 comprises a V_{H} region having at least 90% (e.g., 91%, 92%, 93%, 94%, 95%, 96%, 97%, 98%, 99%, or 100%) identity to the sequence of SEQ ID NO:108. In some embodiments, the Fab that specifically binds to the subdomain IV of human HER2 comprises a V_{H} region having at least 90% (e.g., 91%, 92%, 93%, 94%, 95%, 96%, 97%, 98%, 99%, or 100%) identity to the sequence of SEQ ID NO:109.

In some embodiments, the second Fc polypeptide of the bispecific protein is fused at the N-terminus to an scFv fragment. In some embodiments, the second Fc polypeptide is fused at the N-terminus to the scFv fragment via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. Various linkers are described in detail further herein. In some embodiments, the first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the scFv in the bispecific protein comprises a V_{H} region and a V_{L} region that are connected via a second linker. In some embodiments, the orientation of the V_{L} region and the V_{H} region in the scFv is (N-terminus)-V_{L} region-V_{H} region-(C-terminus), in which the C-terminus of the scFv is joined to the N-terminus of the Fc polypeptide via the first linker. In other embodiments, the orientation of the V_{L} region and the V_{H} region in the scFv is (N-terminus)-V_{H} region-V_{L} region-(C-terminus), in which the C-terminus of the scFv is joined to N-terminus of the Fc polypeptide via the first linker.

In some embodiments, the V_{L} region and the V_{H} region of the scFv are connected via a second linker. In some embodiments, the second linker has a length from about 10 to about 25 amino acids, e.g., from about 10 to about 20, from about 12 to about 25, from about 12 to about 20, from about 14 to about 25, or from about 14 to about 20 amino acids. In some embodiments, the second linker has a length of about 10, 11, 12, 13, 14, 15, 16, 17, 18, 19, 20, 21, 22, 23, 24, or 25 amino acids. In some embodiments, the second linker comprises a flexible linker. Various linkers are described in detail further herein. In some embodiments, the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the V_{L} region and the V_{H} region of the scFv both contain Cys substitutions. In some embodiments, Cys substitutions in the V_{L} region and the V_{H} region can form a disulfide bond and help to stabilize the structure of the scFv. In some embodiments, the scFv comprises a cysteine at each of positions V_{H}44 and V_{L}100, according to Kabat variable domain numbering. In some embodiments, the scFv comprises a disulfide bond between the cysteines at positions V_{H}44 and V_{L}100.

For example, an anti-HER2DII V_{L} region can have a Gln to Cys substitution at position 100 of SEQ ID NO:110. In particular embodiments, the anti-HER2DII V_{L} region containing the Cys substitution can have the sequence of SEQ ID NO:114. In some embodiments, an anti-HER2DIV V_{L} region can have a Gln to Cys substitution at position 100 of SEQ ID NO:111. In particular embodiments, the anti-HER2DIV V_{L} region containing the Cys substitution can have the sequence of SEQ ID NO:115.

For example, an anti-HER2DII V_{H} region can have a Gly to Cys substitution at position 44 of SEQ ID NO:108. In particular embodiments, the anti-HER2DII V_{H} region containing the Cys substitution can have the sequence of SEQ ID NO:112. In some embodiments, an anti-HER2DIV V_{H} region can have a Gly to Cys substitution at position 44 of SEQ ID NO:109. In particular embodiments, the anti-HER2DIV V_{H} region containing the Cys substitution can have the sequence of SEQ ID NO:113.

In some embodiments, in part (a) of the bispecific protein having the structure “Fab-Fc polypeptide/scFv-Fc polypeptide,” the first Fc polypeptide is fused at the N-terminus to the Fd portion of a Fab via a hinge region or a partial hinge region. In some embodiments, in part (b) of the bispecific protein having the structure “Fab-Fc polypeptide/scFv-Fc polypeptide,” the second Fc polypeptide is fused at the N-terminus to the scFv via a hinge region or a partial hinge region. An illustrative hinge region sequence is the human IgG1 hinge sequence EPKSCDKTHTCPPCP (SEQ ID NO:127). A partial hinge region refers to a portion of the sequence of SEQ ID NO:127, for example, a partial hinge region having the sequence of DKTHTCPPCP (SEQ ID NO:128).

In further embodiments, in part (b) of the bispecific protein having the structure “Fab-Fc polypeptide/scFv-Fc polypeptide,” a hinge region (e.g., SEQ ID NO:127) or a partial hinge region (e.g., SEQ ID NO:128) is fused at N-terminus of the second Fc polypeptide. In certain embodiments, when a hinge region is fused at N-terminus of the second Fc polypeptide, the hinge region can contain a Cys to Ser mutation at position 5, relative to the sequence of SEQ ID NO:127. For example, a hinge region having the Cys to Ser mutation can have the sequence of EPKSSDKTHTCPPCP (SEQ ID NO:129).

In the bispecific protein having the structure “Fab-Fc polypeptide/scFv-Fc polypeptide,” the first Fc polypeptide and/or the second Fc polypeptide can specifically bind to a transferrin receptor (e.g., a TfR-binding Fc polypeptide). Different Fc polypeptides and the modifications thereof are described in detail further herein. In some embodiments, the first Fc polypeptide and the second Fc polypeptide can each comprise modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In particular embodiments of the bispecific protein having the structure “Fab-Fc polypeptide/scFv-Fc polypeptide,” the first Fc polypeptide (or the second Fc polypeptide) is a TfR-binding Fc polypeptide that comprises a T366W substitution, L234A and L235A substitutions (optionally including a P329G or a P329S substitution), and optionally a S239D and/or a I332E substitution, according to EU numbering, and the second Fc polypeptide (or the first Fc polypeptide) comprises T366S, L368A, and Y407V substitutions and optionally a S239D and/or a I332E substitution, according to EU numbering. For example, the first Fc polypeptide (or the second Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:137 and 186-196, and the second Fc polypeptide (or the first Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:133 and 183-185).

In certain embodiments, the first Fc polypeptide is a TfR-binding Fc polypeptide and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain embodiments, the first Fc polypeptide does not include the L234A or L325A substitutions (or the P329G or P329S substitution if present in the second Fc polypeptide) and the second Fc polypeptide is a TfR-binding Fc polypeptide and contains L234A and L235A substitutions (optionally including a P329G or a P329S substitution), according to EU numbering.

### Exemplary Bispecific “Fab-Fc Polypeptide/scFv-Fc Polypeptide” Proteins

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to a single-chain variable fragment (scFv) that binds to subdomain IV of human HER2, wherein the first and second Fc polypeptides form an Fc dimer; and
(c) a light chain polypeptide that pairs with the Fd portion recited in (a) to form a Fab.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:159, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:164, (b) comprises a sequence having at least 90% (e.g., 91%, 92%, 93%, 94%, 95%, 96%, 97%, 98%, 99%, or 100%) identity to the sequence of SEQ ID NO:173, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:164, (b) comprises a sequence having at least 90% (e.g., 91%, 92%, 93%, 94%, 95%, 96%, 97%, 98%, 99%, or 100%) identity to the sequence of SEQ ID NO:174, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:166, (b) comprises a sequence having at least 90% (e.g., 91%, 92%, 93%, 94%, 95%, 96%, 97%, 98%, 99%, or 100%) identity to the sequence of SEQ ID NO:173, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:165, (b) comprises a sequence having at least 90% (e.g., 91%, 92%, 93%, 94%, 95%, 96%, 97%, 98%, 99%, or 100%) identity to the sequence of SEQ ID NO:173, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:165, (b) comprises a sequence having at least 90% (e.g., 91%, 92%, 93%, 94%, 95%, 96%, 97%, 98%, 99%, or 100%) identity to the sequence of SEQ ID NO:174, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:167, (b) comprises a sequence having at least 90% (e.g., 91%, 92%, 93%, 94%, 95%, 96%, 97%, 98%, 99%, or 100%) identity to the sequence of SEQ ID NO:173, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:164, (b) comprises a sequence having at least 90% (e.g., 91%, 92%, 93%, 94%, 95%, 96%, 97%, 98%, 99%, or 100%) identity to the sequence of SEQ ID NO:175, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:164, (b) comprises a sequence having at least 90% (e.g., 91%, 92%, 93%, 94%, 95%, 96%, 97%, 98%, 99%, or 100%) identity to the sequence of SEQ ID NO:176, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:166, (b) comprises a sequence having at least 90% (e.g., 91%, 92%, 93%, 94%, 95%, 96%, 97%, 98%, 99%, or 100%) identity to the sequence of SEQ ID NO:175, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:165, (b) comprises a sequence having at least 90% (e.g., 91%, 92%, 93%, 94%, 95%, 96%, 97%, 98%, 99%, or 100%) identity to the sequence of SEQ ID NO:175, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:165, (b) comprises a sequence having at least 90% (e.g., 91%, 92%, 93%, 94%, 95%, 96%, 97%, 98%, 99%, or 100%) identity to the sequence of SEQ ID NO:176, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:167, (b) comprises a sequence having at least 90% (e.g., 91%, 92%, 93%, 94%, 95%, 96%, 97%, 98%, 99%, or 100%) identity to the sequence of SEQ ID NO:175, and (c) comprises a sequence having at least 90% (e.g., 91%, 92%, 93%, 94%, 95%, 96%, 97%, 98%, 99%, or 100%) identity to the sequence of SEQ ID NO:25.

In another example, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:21 or 22, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:20, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:3, (b) comprises a sequence having at least 90% (e.g., 91%, 92%, 93%, 94%, 95%, 96%, 97%, 98%, 99%, or 100%) identity to the sequence of SEQ ID NO:19, and (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:4 or 5, (b) comprises a sequence having at least 90% (e.g., 91%, 92%, 93%, 94%, 95%, 96%, 97%, 98%, 99%, or 100%) identity to the sequence of SEQ ID NO:18, and (c) comprises a sequence having at least 90% (e.g., 91%, 92%, 93%, 94%, 95%, 96%, 97%, 98%, 99%, or 100%) identity to the sequence of SEQ ID NO:25.

In yet another example, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:23, and (c) comprises a sequence having at least 90% (e.g., 91%, 92%, 93%, 94%, 95%, 96%, 97%, 98%, 99%, or 100%) identity to the sequence of SEQ ID NO:25. In yet another example, (a) comprises a sequence having at least 90% (e.g., 91%, 92%, 93%, 94%, 95%, 96%, 97%, 98%, 99%, or 100%) identity to the sequence of SEQ ID NO:5, (b) comprises a sequence having at least 90% (e.g., 91%, 92%, 93%, 94%, 95%, 96%, 97%, 98%, 99%, or 100%) identity to the sequence of SEQ ID NO:24, and (c) comprises a sequence having at least 90% (e.g., 91%, 92%, 93%, 94%, 95%, 96%, 97%, 98%, 99%, or 100%) identity to the sequence of SEQ ID NO:25. Note that in both of these examples, the scFv portion contains an anti-HER2DIV V_{L} region having the Gln to Cys substitution (SEQ ID NO:115) and an anti-HER2DIV V_{H} region having the Gly to Cys substitution (SEQ ID NO: 113). Also in both constructs, the hinge region in (b) has the Cys to Ser mutation at position 5 (EPKSSDKTHTCPPCP (SEQ ID NO:129)).

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to a single-chain variable fragment (scFv) that binds to subdomain II of human HER2, wherein the first and second Fc polypeptides form an Fc dimer; and
(c) a light chain polypeptide that pairs with the Fd portion recited in (a) to form a Fab.

In one embodiment, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:15, 16, or 17, and (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO: 14, and (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:8, (b) comprises a sequence having at least 90% (e.g., 91%, 92%, 93%, 94%, 95%, 96%, 97%, 98%, 99%, or 100%) identity to the sequence of SEQ ID NO:13, and (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:9 or 10, (b) comprises a sequence having at least 90% (e.g., 91%, 92%, 93%, 94%, 95%, 96%, 97%, 98%, 99%, or 100%) identity to the sequence of SEQ ID NO:11 or 12, and (c) comprises a sequence having at least 90% (e.g., 91%, 92%, 93%, 94%, 95%, 96%, 97%, 98%, 99%, or 100%) identity to the sequence of SEQ ID NO:26.

In some embodiments of the bispecific protein described above, the first Fc polypeptide comprises Leu at positions 234 and 235, according to EU numbering. In some embodiments, the second Fc polypeptide comprises Leu at positions 234 and 235, according to EU numbering. In certain embodiments, the protein includes the cis-LALA configuration described below.

In some embodiments of the bispecific protein described above, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the bispecific proteins.

mAb/N-Terminal or C-Terminal scFv on HC

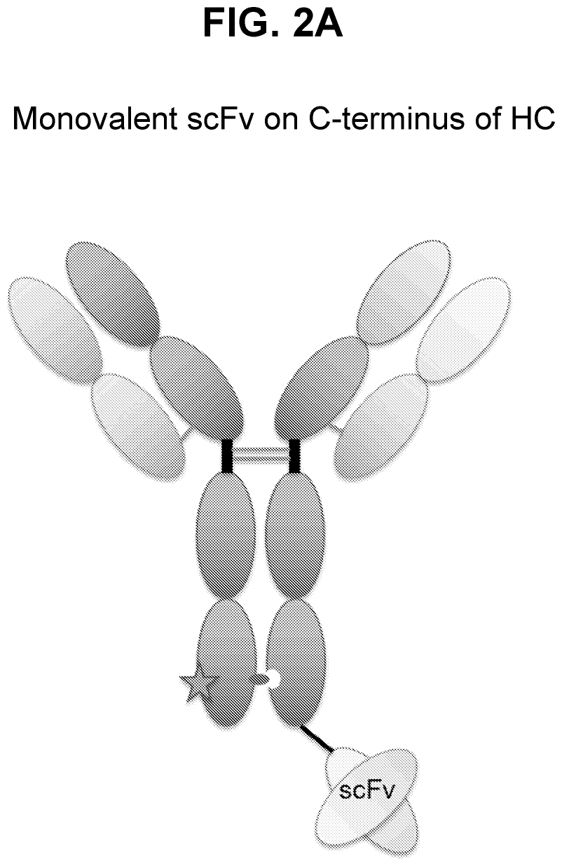

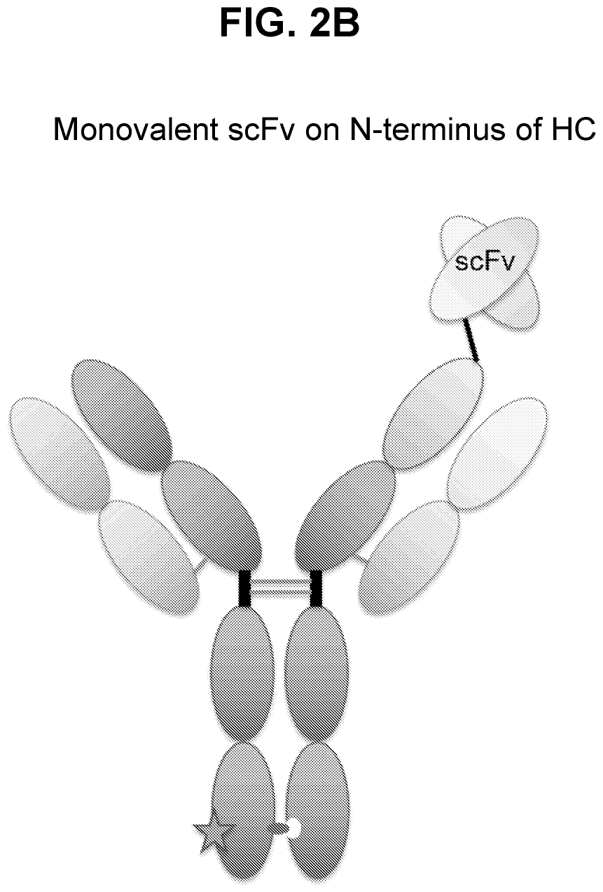

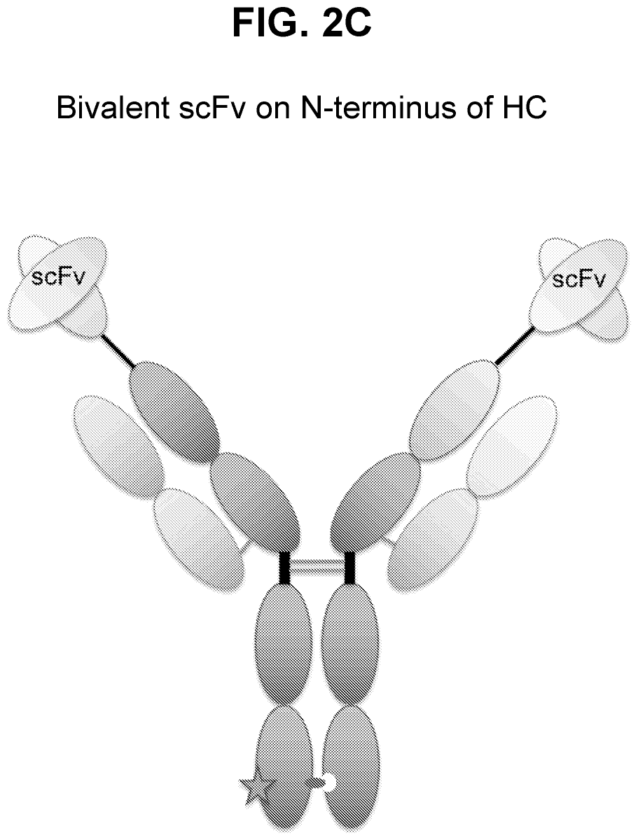

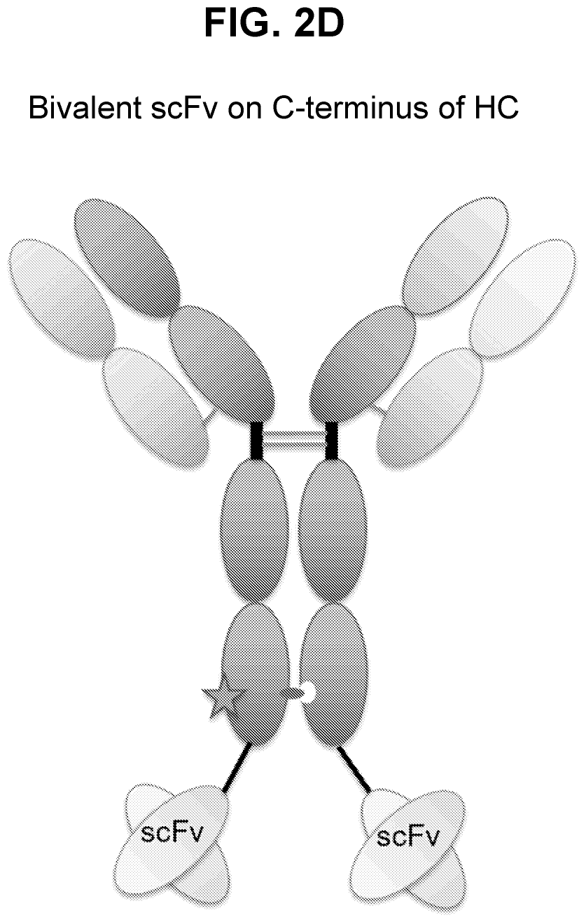

In some embodiments, a bispecific protein comprises Fc polypeptides that are fused at each N-terminus to a Fab that specifically binds to subdomain II or IV of human HER2 and one or both Fc polypeptides are fused at the C-terminus to an scFv that specifically binds to subdomain II or IV of human HER2. In some embodiments, a bispecific protein comprises Fc polypeptides that are fused at each N-terminus to a Fab that specifically binds to subdomain II or IV of human HER2 and one or both Fabs are fused at the N-terminus to an scFv that specifically binds to subdomain II or IV of human HER2. Schematic drawings of such a bispecific protein are shown in FIGS. 2A-2D. In some embodiments, a bispecific protein comprises:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab,
wherein the first Fc polypeptide and/or the second Fc polypeptide is fused at the C-terminus to an scFv, or
wherein the Fd portion in (a) and/or (b) is fused at the N-terminus to an scFv, or
wherein the first Fc polypeptide or the second Fc polypeptide is fused at the C-terminus to an scFv and the Fd portion in (a) or (b) is fused at the N-terminus to an scFv, and
wherein the Fab binds to subdomain II of human HER2 and the scFv binds to subdomain IV of human HER2, or wherein the Fab binds to subdomain IV of human HER2 and the scFv binds to subdomain II of human HER2. In some embodiments, the Fab binds to subdomain II of human HER2 and the scFv binds to subdomain IV of human HER2. In other embodiments, the Fab binds to subdomain IV of human HER2 and the scFv binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

In some embodiments, the Fab that specifically binds to the subdomain II of human HER2 comprises a V_{H} region having at least 90% (e.g., 91%, 92%, 93%, 94%, 95%, 96%, 97%, 98%, 99%, or 100%) identity to the sequence of SEQ ID NO: 108. In some embodiments, the Fab that specifically binds to the subdomain IV of human HER2 comprises a V_{H} region having at least 90% (e.g., 91%, 92%, 93%, 94%, 95%, 96%, 97%, 98%, 99%, or 100%) identity to the sequence of SEQ ID NO:109.

In certain embodiments, the first Fc polypeptide and/or the second Fc polypeptide is fused at the C-terminus to the scFv. In particular embodiments, the first Fc polypeptide or the second Fc polypeptide is fused at the C-terminus to the scFv. In particular embodiments, each of the first Fc polypeptide and the second Fc polypeptide is fused at the C-terminus to the scFv. In certain embodiments, the Fd portion in (a) and/or (b) is fused at the N-terminus to the scFv. In particular embodiments, the Fd portion in (a) or (b) is fused at the N-terminus to the scFv. In particular embodiments, each of the Fd portion in (a) and (b) is fused at the N-terminus to the scFv. In further embodiments, the first Fc polypeptide or the second Fc polypeptide is fused at the C-terminus to the scFv and the Fd portion in (a) or (b) is fused at the N-terminus to the scFv.

In some embodiments, when the first Fc polypeptide and the second Fc polypeptide are each fused at the C-terminus to the scFv, the two scFvs can comprise identical sequences. In some embodiments, when the Fd portion in (a) and the Fd portion in (b) are each fused at the N-terminus to the scFv, the two scFvs can comprise identical sequences. In some embodiments, when the first Fc polypeptide or the second Fc polypeptide is fused at the C-terminus to the scFv and the Fd portion in (a) or (b) is fused at the N-terminus to the scFv, the two scFvs can comprise identical sequences.

In some embodiments, the first Fc polypeptide and/or the second Fc polypeptide is fused at the C-terminus to the scFv via a first linker. In other embodiments, the Fd portion in (a) and/or the Fd portion in (b) is fused at the N-terminus to the scFv via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. Various linkers are described in detail further herein. In certain embodiments, the first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the scFv in the bispecific protein comprises a V_{H} region and a V_{L} region that are connected via a second linker. In some embodiments, the orientation of the V_{L} region and the V_{H} region in the scFv is (N-terminus)-V_{L} region-V_{H} region-(C-terminus), in which the C-terminus of the scFv is joined to the N-terminus of the Fd portion in (a) or (b) via the first linker. In other embodiments, the orientation of the V_{L} region and the V_{H} region in the scFv is (N-terminus)-V_{H} region-V_{L} region-(C-terminus), in which the C-terminus of the scFv is joined to the N-terminus of the Fd portion in (a) or (b) via the first linker. In some embodiments, the orientation of the V_{L} region and the V_{H} region in the scFv is (N-terminus)-V_{L} region-V_{H} region-(C-terminus), in which the N-terminus of the scFv is joined to the C-terminus of the first Fc polypeptide or the second Fc polypeptide via the first linker. In other embodiments, the orientation of the V_{L} region and the V_{H} region in the scFv is (N-terminus)-V_{H} region-V_{L} region-(C-terminus), in which the N-terminus of the scFv is joined to the C-terminus of the first Fc polypeptide or the second Fc polypeptide via the first linker.

In some embodiments, the second linker that connects the V_{L} and V_{H} regions in the scFv has a length from about 10 to about 25 amino acids, e.g., from about 10 to about 20, from about 12 to about 25, from about 12 to about 20, from about 14 to about 25, or from about 14 to about 20 amino acids. In some embodiments, the second linker has a length of about 10, 11, 12, 13, 14, 15, 16, 17, 18, 19, 20, 21, 22, 23, 24, or 25 amino acids. In some embodiments, the second linker comprises a flexible linker. Various linkers are described in detail further herein. In some embodiments, the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the V_{L} region and the V_{H} region of the scFv both contain Cys substitutions. In some embodiments, Cys substitutions in the V_{L} region and the V_{H} region can form a disulfide bond and help to stabilize the structure of the scFv. In some embodiments, the scFv comprises a cysteine at each of positions V_{H}44 and V_{L}100, according to Kabat variable domain numbering. In some embodiments, the scFv comprises a disulfide bond between the cysteines at positions V_{H}44 and V_{L}100.

For example, an anti-HER2DII V_{L} region can have a Gln to Cys substitution at position 100 of SEQ ID NO:110. In particular embodiments, the anti-HER2DII V_{L} region containing the Cys substitution can have the sequence of SEQ ID NO:114. In some embodiments, an anti-HER2DIV V_{L} region can have a Gln to Cys substitution at position 100 of SEQ ID NO:111. In particular embodiments, the anti-HER2DIV V_{L} region containing the Cys substitution can have the sequence of SEQ ID NO:115.

For example, an anti-HER2DII V_{H} region can have a Gly to Cys substitution at position 44 of SEQ ID NO:108. In particular embodiments, the anti-HER2DII V_{H} region containing the Cys substitution can have the sequence of SEQ ID NO:112. In some embodiments, an anti-HER2DIV V_{H} region can have a Gly to Cys substitution at position 44 of SEQ ID NO:109. In particular embodiments, the anti-HER2DIV V_{H} region containing the Cys substitution can have the sequence of SEQ ID NO:113.

In some embodiments, in part (a) of the bispecific protein having the structure “mAb/N-terminal or C-terminal scFv on HC,” the first Fc polypeptide is fused at the N-terminus to the Fd portion of a Fab via a hinge region or a partial hinge region. In some embodiments, in part (b) of the bispecific protein having the structure “mAb/N-terminal or C-terminal scFv on HC,” the second Fc polypeptide is fused at the N-terminus to the Fd portion of a Fab via a hinge region or a partial hinge region. An illustrative hinge region sequence is the human IgG1 hinge sequence EPKSCDKTHTCPPCP (SEQ ID NO:127). A partial hinge region refers to a portion of the sequence of SEQ ID NO:127, for example, a partial hinge region having the sequence of DKTHTCPPCP (SEQ ID NO:128).

In the bispecific protein having the structure “mAb/N-terminal or C-terminal scFv on HC,” the first Fc polypeptide and/or the second Fc polypeptide can specifically bind to a transferrin receptor (e.g., a TfR-binding Fc polypeptide). Different Fc polypeptides and the modifications thereof are described in detail further herein. In some embodiments, the first Fc polypeptide and the second Fc polypeptide can each comprise modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In particular embodiments of the bispecific protein having the structure “mAb/N-terminal or C-terminal scFv on HC,” the first Fc polypeptide (or the second Fc polypeptide) is a TfR-binding Fc polypeptide that comprises a T366W substitution, L234A and L235A substitutions (optionally including a P329G or a P329S substitution), and optionally a S239D and/or a I332E substitution, according to EU numbering, and the second Fc polypeptide (or the first Fc polypeptide) comprises T366S, L368A, and Y407V substitutions and optionally a S239D and/or a I332E substitution, according to EU numbering. For example, the first Fc polypeptide (or the second Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:137 and 186-196, and the second Fc polypeptide (or the first Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:133 and 183-185).

In certain embodiments, the first Fc polypeptide is a TfR-binding Fc polypeptide and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain embodiments, the first Fc polypeptide does not include the L234A or L325A substitutions (or the P329G or P329S substitution if present in the second Fc polypeptide) and the second Fc polypeptide is a TfR-binding Fc polypeptide and contains L234A and L235A substitutions (optionally including a P329G or a P329S substitution), according to EU numbering.

Exemplary Bispecific “mAb/N-Terminal or C-Terminal scFv on HC” Proteins

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein the first Fc polypeptide or the second Fc polypeptide is fused at the C-terminus to an scFv that binds to subdomain IV of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:27 or 28, (b) comprises a sequence having at least 90% (e.g., 91%, 92%, 93%, 94%, 95%, 96%, 97%, 98%, 99%, or 100%) identity to the sequence of SEQ ID NO:4 or 5, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:29, (b) comprises a sequence having at least 90% (e.g., 91%, 92%, 93%, 94%, 95%, 96%, 97%, 98%, 99%, or 100%) identity to the sequence of SEQ ID NO:3, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:30, (b) comprises a sequence having at least 90% (e.g., 91%, 92%, 93%, 94%, 95%, 96%, 97%, 98%, 99%, or 100%) identity to the sequence of SEQ ID NO:2, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In yet another example, (a) comprises a sequence having at least 90% (e.g., 91%, 92%, 93%, 94%, 95%, 96%, 97%, 98%, 99%, or 100%) identity to the sequence of SEQ ID NO:31 or 32, (b) comprises a sequence having at least 90% (e.g., 91%, 92%, 93%, 94%, 95%, 96%, 97%, 98%, 99%, or 100%) identity to the sequence of SEQ ID NO:1, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fe dimer; and
(c) two light chain polypeptides that each pairs with each of the Fd portions recited in (a) and (b) to form the Fab,
   wherein the first Fc polypeptide or the second Fc polypeptide is fused at the C-terminus to an scFv that binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:33 or 34, (b) comprises a sequence having at least 90% (e.g., 91%, 92%, 93%, 94%, 95%, 96%, 97%, 98%, 99%, or 100%) identity to the sequence of SEQ ID NO:9 or 10, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:35, (b) comprises a sequence having at least 90% (e.g., 91%, 92%, 93%, 94%, 95%, 96%, 97%, 98%, 99%, or 100%) identity to the sequence of SEQ ID NO:8, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:36, (b) comprises a sequence having at least 90% (e.g., 91%, 92%, 93%, 94%, 95%, 96%, 97%, 98%, 99%, or 100%) identity to the sequence of SEQ ID NO:7, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:37, 38, or 39, (b) comprises a sequence having at least 90% (e.g., 91%, 92%, 93%, 94%, 95%, 96%, 97%, 98%, 99%, or 100%) identity to the sequence of SEQ ID NO:6, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein each of the first Fc polypeptide and the second Fc polypeptide is fused at the C-terminus to an scFv that binds to subdomain IV of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:27 or 28, (b) comprises a sequence having at least 90% (e.g., 91%, 92%, 93%, 94%, 95%, 96%, 97%, 98%, 99%, or 100%) identity to the sequence of SEQ ID NO:31 or 32, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:29, (b) comprises a sequence having at least 90% (e.g., 91%, 92%, 93%, 94%, 95%, 96%, 97%, 98%, 99%, or 100%) identity to the sequence of SEQ ID NO:30, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein each of the first Fc polypeptide and the second Fc polypeptide is fused at the C-terminus to an scFv that binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:33 or 34, (b) comprises a sequence having at least 90% (e.g., 91%, 92%, 93%, 94%, 95%, 96%, 97%, 98%, 99%, or 100%) identity to the sequence of SEQ ID NO:37, 38, or 39, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:35, (b) comprises a sequence having at least 90% (e.g., 91%, 92%, 93%, 94%, 95%, 96%, 97%, 98%, 99%, or 100%) identity to the sequence of SEQ ID NO:36, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein the Fd portion in (a) or (b) is fused at the N-terminus to an scFv that binds to subdomain IV of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:40, (b) comprises a sequence having at least 90% (e.g., 91%, 92%, 93%, 94%, 95%, 96%, 97%, 98%, 99%, or 100%) identity to the sequence of SEQ ID NO:4 or 5, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:41, (b) comprises a sequence having at least 90% (e.g., 91%, 92%, 93%, 94%, 95%, 96%, 97%, 98%, 99%, or 100%) identity to the sequence of SEQ ID NO:3, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:42, (b) comprises a sequence having at least 90% (e.g., 91%, 92%, 93%, 94%, 95%, 96%, 97%, 98%, 99%, or 100%) identity to the sequence of SEQ ID NO:2, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In yet another example, (a) comprises a sequence having at least 90% (e.g., 91%, 92%, 93%, 94%, 95%, 96%, 97%, 98%, 99%, or 100%) identity to the sequence of SEQ ID NO:43 or 44, (b) comprises a sequence having at least 90% (e.g., 91%, 92%, 93%, 94%, 95%, 96%, 97%, 98%, 99%, or 100%) identity to the sequence of SEQ ID NO:1, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein the Fd portion in (a) or (b) is fused at the N-terminus to an scFv that binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:160, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:161, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:162, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:177, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:178, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:171, (b) comprises a sequence having at least 90% (e.g., 91%, 92%, 93%, 94%, 95%, 96%, 97%, 98%, 99%, or 100%) identity to the sequence of SEQ ID NO:177, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:177, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:178, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:172, (b) comprises a sequence having at least 90% (e.g., 91%, 92%, 93%, 94%, 95%, 96%, 97%, 98%, 99%, or 100%) identity to the sequence of SEQ ID NO:177, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:179, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:180, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:171, (b) comprises a sequence having at least 90% (e.g., 91%, 92%, 93%, 94%, 95%, 96%, 97%, 98%, 99%, or 100%) identity to the sequence of SEQ ID NO:179, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:179, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:180, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:172, (b) comprises a sequence having at least 90% (e.g., 91%, 92%, 93%, 94%, 95%, 96%, 97%, 98%, 99%, or 100%) identity to the sequence of SEQ ID NO:179, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:181, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:169, (b) comprises a sequence having at least 90% (e.g., 91%, 92%, 93%, 94%, 95%, 96%, 97%, 98%, 99%, or 100%) identity to the sequence of SEQ ID NO:182, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:171, (b) comprises a sequence having at least 90% (e.g., 91%, 92%, 93%, 94%, 95%, 96%, 97%, 98%, 99%, or 100%) identity to the sequence of SEQ ID NO:181, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:181, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:170, (b) comprises a sequence having at least 90% (e.g., 91%, 92%, 93%, 94%, 95%, 96%, 97%, 98%, 99%, or 100%) identity to the sequence of SEQ ID NO:182, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:172, (b) comprises a sequence having at least 90% (e.g., 91%, 92%, 93%, 94%, 95%, 96%, 97%, 98%, 99%, or 100%) identity to the sequence of SEQ ID NO:181, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In another example of the bispecific protein, (a) comprises a sequence having at least 90% (e.g., 91%, 92%, 93%, 94%, 95%, 96%, 97%, 98%, 99%, or 100%) identity to the sequence of SEQ ID NO:45 or 46, (b) comprises a sequence having at least 90% (e.g., 91%, 92%, 93%, 94%, 95%, 96%, 97%, 98%, 99%, or 100%) identity to the sequence of SEQ ID NO:9 or 10, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:47, (b) comprises a sequence having at least 90% (e.g., 91%, 92%, 93%, 94%, 95%, 96%, 97%, 98%, 99%, or 100%) identity to the sequence of SEQ ID NO:8, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:48, (b) comprises a sequence having at least 90% (e.g., 91%, 92%, 93%, 94%, 95%, 96%, 97%, 98%, 99%, or 100%) identity to the sequence of SEQ ID NO:7, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:49 or 50, (b) comprises a sequence having at least 90% (e.g., 91%, 92%, 93%, 94%, 95%, 96%, 97%, 98%, 99%, or 100%) identity to the sequence of SEQ ID NO:6, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein each of the Fd portion in (a) and (b) is fused at the N-terminus to an scFv that binds to subdomain IV of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:40, (b) comprises a sequence having at least 90% (e.g., 91%, 92%, 93%, 94%, 95%, 96%, 97%, 98%, 99%, or 100%) identity to the sequence of SEQ ID NO:43 or 44, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:41, (b) comprises a sequence having at least 90% (e.g., 91%, 92%, 93%, 94%, 95%, 96%, 97%, 98%, 99%, or 100%) identity to the sequence of SEQ ID NO:42, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein each of the Fd portion in (a) and (b) is fused at the N-terminus to an scFv that binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:45, (b) comprises a sequence having at least 90% (e.g., 91%, 92%, 93%, 94%, 95%, 96%, 97%, 98%, 99%, or 100%) identity to the sequence of SEQ ID NO:49 or 50, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:47, (b) comprises a sequence having at least 90% (e.g., 91%, 92%, 93%, 94%, 95%, 96%, 97%, 98%, 99%, or 100%) identity to the sequence of SEQ ID NO:48, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein the first Fc polypeptide or the second Fc polypeptide is fused at the C-terminus to an scFv that binds to subdomain IV of human HER2 and the Fd portion in (a) or (b) is fused at the N-terminus to the scFv.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:40, (b) comprises a sequence having at least 90% (e.g., 91%, 92%, 93%, 94%, 95%, 96%, 97%, 98%, 99%, or 100%) identity to the sequence of SEQ ID NO:31 or 32, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:41, (b) comprises a sequence having at least 90% (e.g., 91%, 92%, 93%, 94%, 95%, 96%, 97%, 98%, 99%, or 100%) identity to the sequence of SEQ ID NO:30, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:42, (b) comprises a sequence having at least 90% (e.g., 91%, 92%, 93%, 94%, 95%, 96%, 97%, 98%, 99%, or 100%) identity to the sequence of SEQ ID NO:29, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:43 or 44, (b) comprises a sequence having at least 90% (e.g., 91%, 92%, 93%, 94%, 95%, 96%, 97%, 98%, 99%, or 100%) identity to the sequence of SEQ ID NO:27 or 28, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab,
   wherein the first Fc polypeptide or the second Fc polypeptide is fused at the C-terminus to an scFv that binds to subdomain II of human HER2 and the Fd portion in (a) or (b) is fused at the N-terminus to the scFv.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:45, (b) comprises a sequence having at least 90% (e.g., 91%, 92%, 93%, 94%, 95%, 96%, 97%, 98%, 99%, or 100%) identity to the sequence of SEQ ID NO:37, 38, or 39, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:47, (b) comprises a sequence having at least 90% (e.g., 91%, 92%, 93%, 94%, 95%, 96%, 97%, 98%, 99%, or 100%) identity to the sequence of SEQ ID NO:36, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:48, (b) comprises a sequence having at least 90% (e.g., 91%, 92%, 93%, 94%, 95%, 96%, 97%, 98%, 99%, or 100%) identity to the sequence of SEQ ID NO:35, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:49 or 50, (b) comprises a sequence having at least 90% (e.g., 91%, 92%, 93%, 94%, 95%, 96%, 97%, 98%, 99%, or 100%) identity to the sequence of SEQ ID NO:33 or 34, and each of the two light chain polypeptides in (c) comprises a sequence having at least 90% (e.g., 91%, 92%, 93%, 94%, 95%, 96%, 97%, 98%, 99%, or 100%) identity to the sequence of SEQ ID NO:26.

In some embodiments of the bispecific protein described above, the first Fc polypeptide comprises Leu at positions 234 and 235, according to EU numbering. In some embodiments, the second Fc polypeptide comprises Leu at positions 234 and 235, according to EU numbering. In certain embodiments, the protein includes the cis-LALA configuration described below.

In some embodiments of the bispecific protein described above, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the bispecific proteins.

mAb/N-Terminal or C-Terminal scFv on LC

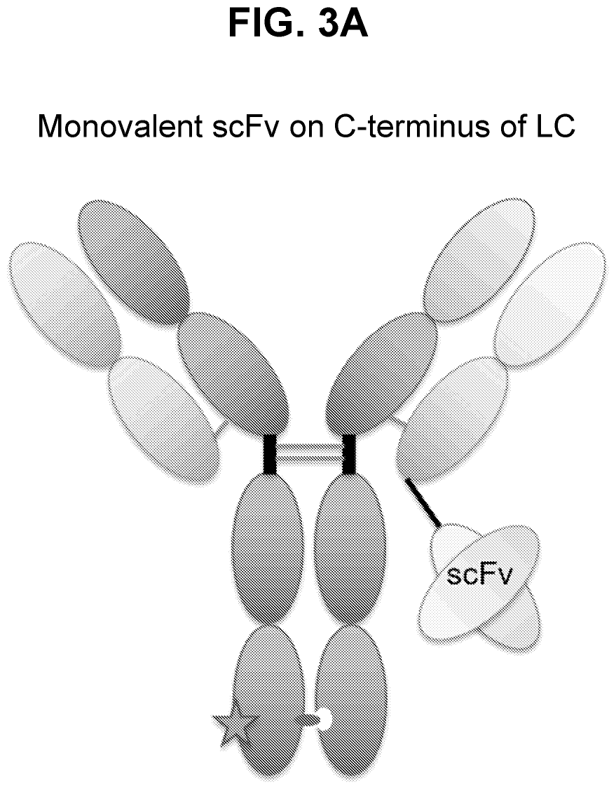

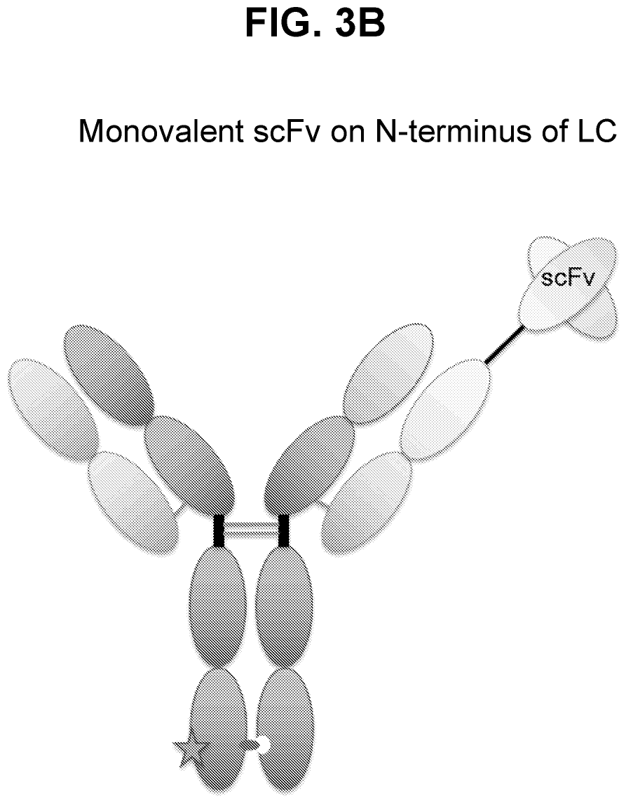

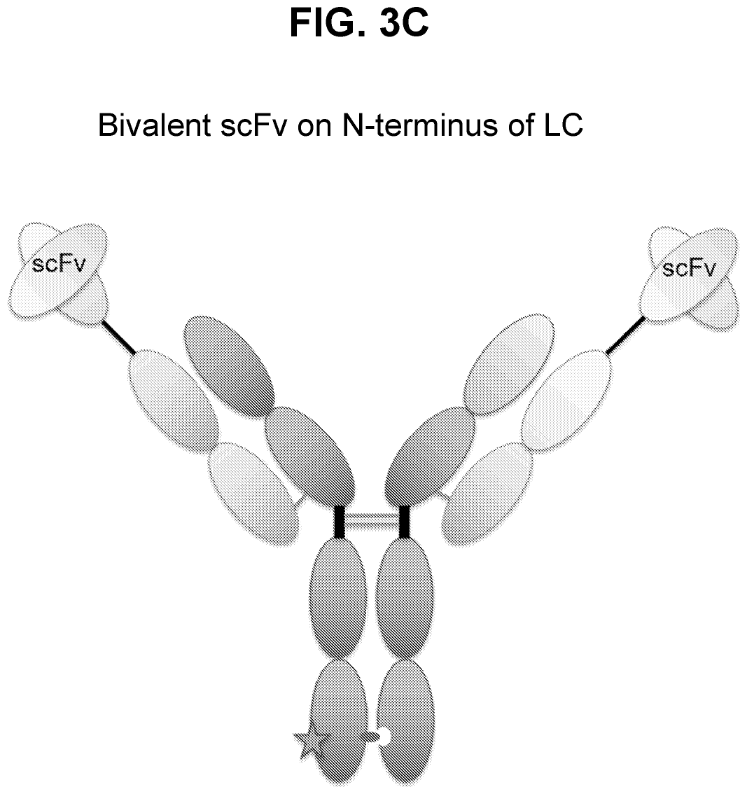

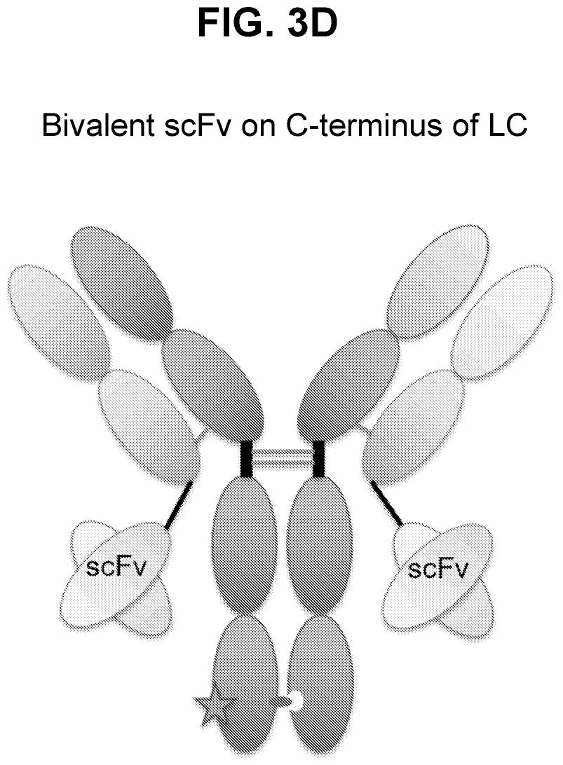

In some embodiments, a bispecific protein comprises light chains that are fused at the N-terminus and/or the C-terminus to an scFv that specifically binds to subdomain II or IV of human HER2. Schematic drawings of such a bispecific protein are shown in FIGS. 3A-3D. In some embodiments, a bispecific protein comprises:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab;
wherein one or both of the light chain polypeptides are fused at the N-terminus to an scFv, or
wherein one or both of the light chain polypeptides are fused at the C-terminus to an scFv, or
wherein a first light chain polypeptide is fused at the N-terminus to an scFv and the second light chain polypeptide is fused at the C-terminus to an scFv, and
wherein the Fab binds to subdomain II of human HER2 and the scFv binds to subdomain IV of human HER2, or wherein Fab binds to subdomain IV of human HER2 and the scFv binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

In some embodiments, the Fab that specifically binds to the subdomain II of human HER2 comprises a V_{H} region having at least 90% (e.g., 91%, 92%, 93%, 94%, 95%, 96%, 97%, 98%, 99%, or 100%) identity to the sequence of SEQ ID NO:108. In some embodiments, the Fab that specifically binds to the subdomain IV of human HER2 comprises a V_{H} region having at least 90% (e.g., 91%, 92%, 93%, 94%, 95%, 96%, 97%, 98%, 99%, or 100%) identity to the sequence of SEQ ID NO:109.

In certain embodiments, one or both of the light chain polypeptides are fused at the N-terminus to the scFv. In particular embodiments, one of the light chain polypeptides is fused at the N-terminus to the scFv. In particular embodiments, each of the two light chain polypeptides is fused at the N-terminus to the scFv. In certain embodiments, one or both of the light chain polypeptides are fused at the C-terminus to the scFv. In particular embodiments, one of the light chain polypeptides is fused at the C-terminus to the scFv. In particular embodiments, each of the two light chain polypeptides is fused at the C-terminus to the scFv. In further embodiments, a first light chain polypeptide is fused at the N-terminus to the scFv and a second light chain polypeptide is fused at the C-terminus to the scFv.

In some embodiments, when both light chain polypeptides are each fused at the C-terminus to the scFv, the two scFvs can comprise identical sequences. In some embodiments, when both light chain polypeptides are each fused at the N-terminus to the scFv, the two scFvs can comprise identical sequences.

In some embodiments, the one or both of the light chain polypeptides are fused to the scFv via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. Various linkers are described in detail further herein. In certain embodiments, the first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the scFv in the bispecific protein comprises a V_{H} region and a V_{L} region that are connected via a second linker. In some embodiments, the orientation of the V_{L} region and the V_{H} region in the scFv is (N-terminus)-V_{L} region-V_{H} region-(C-terminus), in which the C-terminus of the scFv is joined to the N-terminus of the light chain polypeptides via the first linker. In other embodiments, the orientation of the V_{L} region and the V_{H} region in the scFv is (N-terminus)-V_{H} region-V_{L} region-(C-terminus), in which the C-terminus of the scFv is joined to the N-terminus of the light chain polypeptides via the first linker. In some embodiments, the orientation of the V_{L} region and the V_{H} region in the scFv is (N-terminus)-V_{L} region-V_{H} region-(C-terminus), in which the N-terminus of the scFv is joined to the C-terminus of the light chain polypeptides via the first linker. In other embodiments, the orientation of the V_{L} region and the V_{H} region in the scFv is (N-terminus)-V_{H} region-V_{L} region-(C-terminus), in which the N-terminus of the scFv is joined to the C-terminus of the light chain polypeptides via the first linker.

In some embodiments, the second linker that connects the V_{L} and V_{H} regions in the scFv has a length from about 10 to about 25 amino acids, e.g., from about 10 to about 20, from about 12 to about 25, from about 12 to about 20, from about 14 to about 25, or from about 14 to about 20 amino acids. In some embodiments, the second linker has a length of about 10, 11, 12, 13, 14, 15, 16, 17, 18, 19, 20, 21, 22, 23, 24, or 25 amino acids. In some embodiments, the second linker comprises a flexible linker. Various linkers are described in detail further herein. In some embodiments, the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, the V_{L} region and the V_{H} region of the scFv both contain Cys substitutions. In some embodiments, Cys substitutions in the V_{L} region and the V_{H} region can form a disulfide bond and help to stabilize the structure of the scFv. In some embodiments, the scFv comprises a cysteine at each of positions V_{H}44 and V_{L}100, according to Kabat variable domain numbering. In some embodiments, the scFv comprises a disulfide bond between the cysteines at positions V_{H}44 and V_{L}100.

For example, an anti-HER2DII V_{L} region can have a Gln to Cys substitution at position 100 of SEQ ID NO:110. In particular embodiments, the anti-HER2DII V_{L} region containing the Cys substitution can have the sequence of SEQ ID NO:114. In some embodiments, an anti-HER2DIV V_{L} region can have a Gln to Cys substitution at position 100 of SEQ ID NO:111. In particular embodiments, the anti-HER2DIV V_{L} region containing the Cys substitution can have the sequence of SEQ ID NO:115.

For example, an anti-HER2DII V_{H} region can have a Gly to Cys substitution at position 44 of SEQ ID NO:108. In particular embodiments, the anti-HER2DII V_{H} region containing the Cys substitution can have the sequence of SEQ ID NO:112. In some embodiments, an anti-HER2DIV V_{H} region can have a Gly to Cys substitution at position 44 of SEQ ID NO:109. In particular embodiments, the anti-HER2DIV V_{H} region containing the Cys substitution can have the sequence of SEQ ID NO:113.

In some embodiments, in part (a) of the bispecific protein having the structure “mAb/N-terminal or C-terminal scFv on LC,” the first Fc polypeptide is fused at the N-terminus to the Fd portion of a Fab via a hinge region or a partial hinge region. In some embodiments, in part (b) of the bispecific protein having the structure “mAb/N-terminal or C-terminal scFv on LC,” the second Fc polypeptide is fused at the N-terminus to the Fd portion of a Fab via a hinge region or a partial hinge region. An illustrative hinge region sequence is the human IgG1 hinge sequence EPKSCDKTHTCPPCP (SEQ ID NO:127). A partial hinge region refers to a portion of the sequence of SEQ ID NO:127, for example, a partial hinge region having the sequence of DKTHTCPPCP (SEQ ID NO:128).

In the bispecific protein having the structure “mAb/N-terminal or C-terminal scFv on LC,” the first Fc polypeptide and/or the second Fc polypeptide can specifically bind to a transferrin receptor (e.g., a TfR-binding Fc polypeptide). Different Fc polypeptides and the modifications thereof are described in detail further herein. In some embodiments, the first Fc polypeptide and the second Fc polypeptide can each comprise modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In particular embodiments of the bispecific protein having the structure “mAb/N-terminal or C-terminal scFv on HC,” the first Fc polypeptide (or the second Fc polypeptide) is a TfR-binding Fc polypeptide that comprises a T366W substitution, L234A and L235A substitutions (optionally including a P329G or a P329S substitution), and optionally a S239D and/or a I332E substitution, according to EU numbering, and the second Fc polypeptide (or the first Fc polypeptide) comprises T366S, L368A, and Y407V substitutions and optionally a S239D and/or a I332E substitution, according to EU numbering. For example, the first Fc polypeptide (or the second Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:137 and 186-196, and the second Fc polypeptide (or the first Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:133 and 183-185).

In certain embodiments, the first Fc polypeptide is a TfR-binding Fc polypeptide and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain embodiments, the first Fc polypeptide does not include the L234A or L325A substitutions (or the P329G or P329S substitution if present in the second Fc polypeptide) and the second Fc polypeptide is a TfR-binding Fc polypeptide and contains L234A and L235A substitutions (optionally including a P329G or a P329S substitution), according to EU numbering.

Exemplary Bispecific “mAb/N-Terminal or C-Terminal scFv on LC” Proteins

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fe dimer; and
(c) two light chain polypeptides that each pairs with each of the Fd portions recited in (a) and (b) to form the Fab;
wherein one of the light chain polypeptides is fused at the C-terminus to an scFv that binds to subdomain IV of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:4 or 5, a first light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52, and a second light chain polypeptide comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:3, a first light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52, and a second light chain polypeptide comprises a sequence having at least 90% (e.g., 91%, 92%, 93%, 94%, 95%, 96%, 97%, 98%, 99%, or 100%) identity to the sequence of SEQ ID NO:25.

In some embodiments, a bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein one of the light chain polypeptides is fused at the C-terminus to an scFv that binds to subdomain II of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:9 or 10, a first light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54, and a second light chain polypeptide comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO:8, a first light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54, and a second light chain polypeptide comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the two light chain polypeptides is fused at the C-terminus to an scFv that binds to subdomain IV of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:4 or 5, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:3, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52.

In some embodiments, the bispecific protein comprises:
some embodiments, a bispecific protein comprises:
   (a) a first Fc polypeptide that is fused at the N-terminus to an Fd portion of a Fab that binds to subdomain IV of human HER2;
   (b) a second Fc polypeptide that is fused at the N-terminus to an Fd portion of the Fab, wherein the first and second Fc polypeptides form an Fc dimer; and
   (c) two light chain polypeptides that each pairs with each of the Fd portions recited in (a) and (b) to form the Fab;
   wherein each of the two light chain polypeptides is fused at the C-terminus to an scFv that binds to subdomain II of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:9 or 10, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54. In yet another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO:8, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein one of the light chain polypeptides is fused at the N-terminus to an scFv that binds to subdomain IV of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:4 or 5, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:55, and a second light chain polypeptide comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:3, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:55, and a second light chain polypeptide comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fe dimer; and
(c) two light chain polypeptides that each pairs with each of the Fd portions recited in (a) and (b) to form the Fab;
wherein one of the light chain polypeptides is fused at the N-terminus to an scFv that binds to subdomain II of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:9 or 10, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57, and a second light chain polypeptide comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO:8, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57, and a second light chain polypeptide comprises a sequence having at least 90% (e.g., 91%, 92%, 93%, 94%, 95%, 96%, 97%, 98%, 99%, or 100%) identity to the sequence of SEQ ID NO:26.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the two light chain polypeptides is fused at the N-terminus to an scFv that binds to subdomain IV of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:4 or 5, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:55. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:3, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:55.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the two light chain polypeptides is fused at the N-terminus to an scFv that binds to subdomain II of human HER2.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:9 or 10, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57. In another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO:8, and each of the two light chain polypeptides is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein a first light chain polypeptide is fused at the N-terminus to an scFv that binds to subdomain IV of human HER2 and the second light chain polypeptide is fused at the C-terminus to the scFv.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:1, (b) comprises a sequence having at least 90% (e.g., 91%, 92%, 93%, 94%, 95%, 96%, 97%, 98%, 99%, or 100%) identity to the sequence of SEQ ID NO:4 or 5, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:55, and a second light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52. In another example, (a) comprises a sequence having at least 90% (e.g., 91%, 92%, 93%, 94%, 95%, 96%, 97%, 98%, 99%, or 100%) identity to the sequence of SEQ ID NO:2, (b) comprises a sequence having at least 90% (e.g., 91%, 92%, 93%, 94%, 95%, 96%, 97%, 98%, 99%, or 100%) identity to the sequence of SEQ ID NO:3, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:55, and a second light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:52.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein a first light chain polypeptide is fused at the N-terminus to an scFv that binds to subdomain II of human HER2 and the second light chain polypeptide is fused at the C-terminus to the scFv.

In some embodiments, the first Fc polypeptide and/or the second Fc polypeptide independently comprises a S239D and/or I332E a substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:6, (b) comprises a sequence having at least 90% (e.g., 91%, 92%, 93%, 94%, 95%, 96%, 97%, 98%, 99%, or 100%) identity to the sequence of SEQ ID NO:9 or 10, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57, and a second light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54 comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:7, (b) comprises a sequence having at least 90% (e.g., 91%, 92%, 93%, 94%, 95%, 96%, 97%, 98%, 99%, or 100%) identity to the sequence of SEQ ID NO:8, a first light chain polypeptide is fused at the N-terminus to the scFv and comprises a sequence having at least 90% (e.g., 91%, 92%, 93%, 94%, 95%, 96%, 97%, 98%, 99%, or 100%) identity to the sequence of SEQ ID NO:56 or 57, and a second light chain polypeptide is fused at the C-terminus to the scFv and comprises a sequence having at least 90% (e.g., 91%, 92%, 93%, 94%, 95%, 96%, 97%, 98%, 99%, or 100%) identity to the sequence of SEQ ID NO:53 or 54 comprises a sequence having at least 90% (e.g., 91%, 92%, 93%, 94%, 95%, 96%, 97%, 98%, 99%, or 100%) identity to the sequence of SEQ ID NO:26.

In some embodiments of the bispecific protein described above, the first Fc polypeptide comprises Leu at positions 234 and 235, according to EU numbering. In some embodiments, the second Fc polypeptide comprises Leu at positions 234 and 235, according to EU numbering. In certain embodiments, the protein includes the cis-LALA configuration described below.

In some embodiments of the bispecific protein described above, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the bispecific proteins.

mAb/N-Terminal V_{H} V_{L} on HC and LC

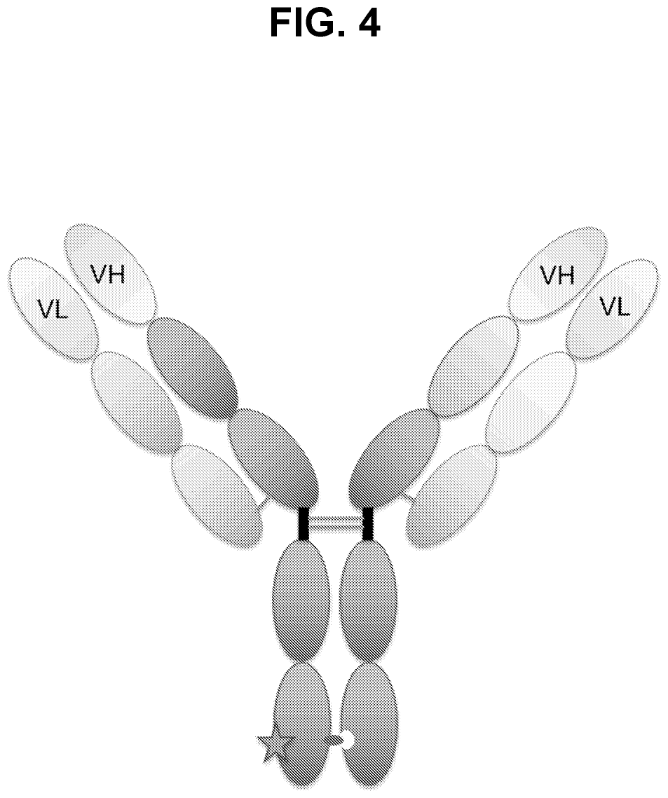

In some embodiments, a bispecific protein comprises a V_{H} region (or a V_{L} region) fused at the N-terminus of each of the two heavy chains and a V_{L} region (or a V_{H} region) fused at the N-terminus of each of the two light chains. A schematic drawing of such a bispecific protein is shown in FIG. 4. In some embodiments, a bispecific protein comprises:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab;
wherein each of the Fd portions in (a) and (b) is fused at the N-terminus to a V_{H} region or a V_{L} region of an Fv fragment, and
wherein each of the two light chain polypeptides is fused at the N-terminus to the other of a V_{H} region or a V_{L} region of the Fv fragment, and
wherein the V_{H} region and the V_{L} region together form the Fv fragment, and
wherein the Fab binds to subdomain II of human HER2 and the Fv fragment binds to subdomain IV of human HER2, or wherein the Fab binds to subdomain IV of human HER2 and the Fv fragment binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

In some embodiments of the bispecific protein, each of the Fd portions in (a) and (b) is fused at the N-terminus to the V_{H} region of an Fv fragment, and each of the two light chain polypeptides is fused at the N-terminus to the V_{L} region of the Fv fragment. In other embodiments, each of the Fd portions in (a) and (b) is fused at the N-terminus to the V_{L} region of an Fv fragment, and each of the two light chain polypeptides is fused at the N-terminus to the V_{H} region of the Fv fragment.

In some embodiments, the Fab that specifically binds to the subdomain II of human HER2 comprises a V_{H} region having at least 90% (e.g., 91%, 92%, 93%, 94%, 95%, 96%, 97%, 98%, 99%, or 100%) identity to the sequence of SEQ ID NO:108. In some embodiments, the Fab that specifically binds to the subdomain IV of human HER2 comprises a V_{H} region having at least 90% (e.g., 91%, 92%, 93%, 94%, 95%, 96%, 97%, 98%, 99%, or 100%) identity to the sequence of SEQ ID NO:109.

In some embodiments, a first linker connects a V_{H} region or a V_{L} region to the N-terminus of each of the Fd portions in (a) and (b). In some embodiments, a second linker connects a V_{H} region or a V_{L} region to the N-terminus of each of the two light chain polypeptides. In some embodiments, the first linker or the second linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. Various linkers are described in detail further herein. In certain embodiments, the first linker comprises a sequence of ASTKGPSVF (SEQ ID NO:125). In certain embodiments, the second linker comprises a sequence of RTVAAPSVFI (SEQ ID NO:126).

In some embodiments, in part (a) of the bispecific protein having the structure “mAb/N-terminal V_{H} V_{L} on HC and LC,” the first Fc polypeptide is fused at the N-terminus to the Fd portion of a Fab via a hinge region or a partial hinge region. In some embodiments, in part (b) of the bispecific protein having the structure “mAb/N-terminal V_{H} V_{L} on HC and LC,” the second Fc polypeptide is fused at the N-terminus to the Fd portion of a Fab via a hinge region or a partial hinge region. An illustrative hinge region sequence is the human IgG1 hinge sequence EPKSCDKTHTCPPCP (SEQ ID NO:127). A partial hinge region refers to a portion of the sequence of SEQ ID NO:127, for example, a partial hinge region having the sequence of DKTHTCPPCP (SEQ ID NO:128).

In the bispecific protein having the structure “mAb/N-terminal V_{H} V_{L} on HC and LC,” the first Fc polypeptide and/or the second Fc polypeptide can specifically bind to a transferrin receptor (e.g., a TfR-binding Fc polypeptide). Different Fc polypeptides and the modifications thereof are described in detail further herein. In some embodiments, the first Fc polypeptide and the second Fc polypeptide can each comprise modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In particular embodiments of the bispecific protein having the structure “mAb/N-terminal V_{H} V_{L} on HC and LC,” the first Fc polypeptide (or the second Fc polypeptide) is a TfR-binding Fc polypeptide that comprises a T366W substitution, L234A and L235A substitutions (optionally including a P329G or a P329S substitution), and optionally a S239D and/or a I332E substitution, according to EU numbering, and the second Fc polypeptide (or the first Fc polypeptide) comprises T366S, L368A, and Y407V substitutions and optionally a S239D and/or a I332E substitution, according to EU numbering. For example, the first Fc polypeptide (or the second Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:137 and 186-196, and the second Fc polypeptide (or the first Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:133 and 183-185).

In certain embodiments, the first Fc polypeptide is a TfR-binding Fc polypeptide and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain embodiments, the first Fc polypeptide does not include the L234A or L325A substitutions (or the P329G or P329S substitution if present in the second Fc polypeptide) and the second Fc polypeptide is a TfR-binding Fc polypeptide and contains L234A and L235A substitutions (optionally including a P329G or a P329S substitution), according to EU numbering.

Exemplary Bispecific “mAb/N-Terminal V_{H} V_{L} on HC and LC” Proteins

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the Fd portions in (a) and (b) is fused at the N-terminus to a V_{H} region of an Fv fragment that binds to subdomain IV of human HER2, and
wherein each of the two light chain polypeptides is fused at the N-terminus to the other of a V_{L} region of the Fv fragment, and
wherein the V_{H} region and the V_{L} region together form the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, the bispecific protein comprises a first polypeptide comprising (a) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:58, a second polypeptide comprising (b) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:61 or 62, and third and fourth polypeptides each comprising a light chain polypeptide fused at the N-terminus to the V_{L} region of the Fv fragment and each comprising a sequence having at least 90% (e.g., 91%, 92%, 93%, 94%, 95%, 96%, 97%, 98%, 99%, or 100%) identity to the sequence of SEQ ID NO:78. In another example, the bispecific protein comprises a first polypeptide comprising (a) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:59, a second polypeptide comprising (b) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:60, and third and fourth polypeptides each comprising a light chain polypeptide fused at the N-terminus to the V_{L} region of the Fv fragment and each comprising a sequence having at least 90% (e.g., 91%, 92%, 93%, 94%, 95%, 96%, 97%, 98%, 99%, or 100%) identity to the sequence of SEQ ID NO:78.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the Fd portions in (a) and (b) is fused at the N-terminus to a V_{H} region of an Fv fragment that binds to subdomain II of human HER2, and
wherein each of the two light chain polypeptides is fused at the N-terminus to the other of a V_{L} region of the Fv fragment, and
wherein the V_{H} region and the V_{L} region together form the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, the bispecific protein comprises a first polypeptide comprising (a) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:63, a second polypeptide comprising (b) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:66 or 67, and third and fourth polypeptides each comprising a light chain polypeptide fused at the N-terminus to the V_{L} region of the Fv fragment and each comprising a sequence having at least 90% (e.g., 91%, 92%, 93%, 94%, 95%, 96%, 97%, 98%, 99%, or 100%) identity to the sequence of SEQ ID NO:79. In another example, a first polypeptide comprising (a) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:64, a second polypeptide comprising (b) fused at the N-terminus to the V_{H} region of the Fv fragment and a sequence having at least 90% (e.g., 91%, 92%, 93%, 94%, 95%, 96%, 97%, 98%, 99%, or 100%) identity to the sequence of SEQ ID NO:65, and third and fourth polypeptides each comprising a light chain polypeptide fused at the N-terminus to the V_{L} region of the Fv fragment and each comprising a sequence having at least 90% (e.g., 91%, 92%, 93%, 94%, 95%, 96%, 97%, 98%, 99%, or 100%) identity to the sequence of SEQ ID NO:79.

In some embodiments, the bispecific protein comprises:
(a) a first Fc polypeptide that is fused at the N-terminus to an Fd portion of a Fab that binds to subdomain II of human HER2;
(b) a second Fc polypeptide that is fused at the N-terminus to an Fd portion of the Fab, wherein the first and second Fc polypeptides form an Fc dimer; and
(c) two light chain polypeptides that each pairs with each of the Fd portions recited in (a) and (b) to form the Fab;
wherein each of the Fd portions in (a) and (b) is fused at the N-terminus to a V_{L} region of an Fv fragment that binds to subdomain IV of human HER2, and
wherein each of the two light chain polypeptides is fused at the N-terminus to the other of a V_{H} region of the Fv fragment, and
wherein the V_{H} region and the V_{L} region together form the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, the bispecific protein comprises a first polypeptide comprising (a) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:68, a second polypeptide comprising (b) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:71 or 72, and third and fourth polypeptides each comprising a light chain polypeptide fused at the N-terminus to the V_{H} region of the Fv fragment and each comprising a sequence having at least 90% (e.g., 91%, 92%, 93%, 94%, 95%, 96%, 97%, 98%, 99%, or 100%) identity to the sequence of SEQ ID NO:80. In another example, the bispecific protein comprises a first polypeptide comprising (a) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:69, a second polypeptide comprising (b) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:70, and third and fourth polypeptides each comprising a light chain polypeptide fused at the N-terminus to the V_{H} region of the Fv fragment and each comprising a sequence having at least 90% (e.g., 91%, 92%, 93%, 94%, 95%, 96%, 97%, 98%, 99%, or 100%) identity to the sequence of SEQ ID NO:80.

In some embodiments, the bispecific protein comprises:
(a) a first Fc polypeptide that is fused at the N-terminus to an Fd portion of a Fab that binds to subdomain IV of human HER2;
(b) a second Fc polypeptide that is fused at the N-terminus to an Fd portion of the Fab, wherein the first and second Fc polypeptides form an Fe dimer; and
(c) two light chain polypeptides that each pairs with each of the Fd portions recited in (a) and (b) to form the Fab;
wherein each of the Fd portions in (a) and (b) is fused at the N-terminus to a V_{L} region of an Fv fragment that binds to subdomain II of human HER2, and
wherein each of the two light chain polypeptides is fused at the N-terminus to the other of a V_{H} region of the Fv fragment, and
wherein the V_{H} region and the V_{L} region together form the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example, the bispecific protein comprises a first polypeptide comprising (a) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:73, a second polypeptide comprising (b) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:76 or 77, and third and fourth polypeptides each comprising a light chain polypeptide fused at the N-terminus to the V_{H} region of the Fv fragment and each comprising a sequence having at least 90% (e.g., 91%, 92%, 93%, 94%, 95%, 96%, 97%, 98%, 99%, or 100%) identity to the sequence of SEQ ID NO:81. In another example, the bispecific protein comprises a first polypeptide comprising (a) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:74, a second polypeptide comprising (b) fused at the N-terminus to the V_{L} region of the Fv fragment and a sequence having at least 90% (e.g., 91%, 92%, 93%, 94%, 95%, 96%, 97%, 98%, 99%, or 100%) identity to the sequence of SEQ ID NO:75, and third and fourth polypeptides each comprising a light chain polypeptide fused at the N-terminus to the V_{H} region of the Fv fragment and each comprising a sequence having at least 90% (e.g., 91%, 92%, 93%, 94%, 95%, 96%, 97%, 98%, 99%, or 100%) identity to the sequence of SEQ ID NO:81.

In some embodiments of the bispecific protein described above, the first Fc polypeptide comprises Leu at positions 234 and 235, according to EU numbering. In some embodiments, the second Fc polypeptide comprises Leu at positions 234 and 235, according to EU numbering. In certain embodiments, the protein includes the cis-LALA configuration described below.

In some embodiments of the bispecific protein described above, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the bispecific proteins.

mAb/C-Terminal V_{H} V_{L} on HC

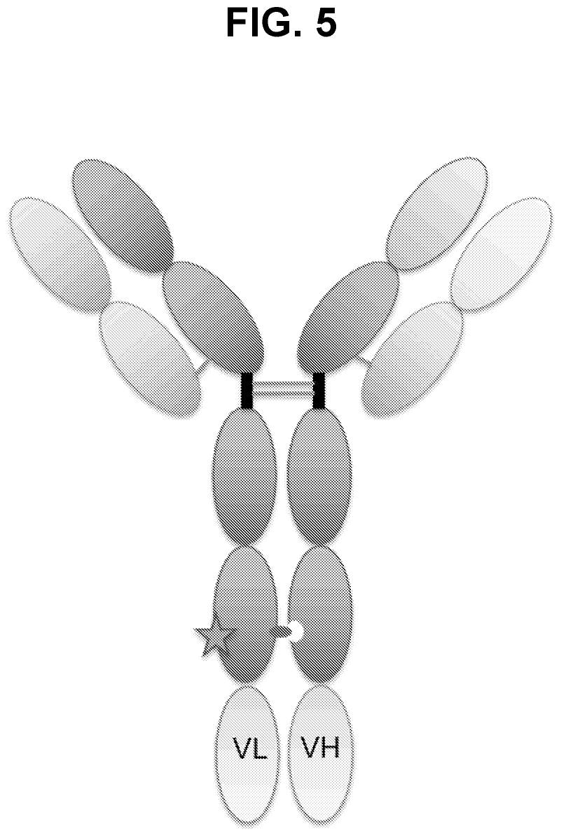

In some embodiments, a bispecific protein comprises a V_{H} region (or a V_{L} region) fused at the C-terminus of one of the two Fc polypeptides and a V_{L} region (or a V_{H} region) fused at the C-terminus of the other of the two Fc polypeptides. A schematic drawing of such a bispecific protein is shown in FIG. 5. In some embodiments, a bispecific protein comprises:
(a) a first Fc polypeptide that is fused at the N-terminus to an Fd portion of a Fab, and is fused at the C-terminus to a V_{H} region or a V_{L} region of an Fv fragment;
(b) a second Fc polypeptide that is fused at the N-terminus to an Fd portion of a Fab, and is fused at the C-terminus to the other of a V_{H} region or a V_{L} region recited in (a),
wherein the V_{H} region and the V_{L} region together form the Fv fragment, and wherein the first and second Fc polypeptides form an Fc dimer; and
(c) two light chain polypeptides that each pairs with each Fd portion recited in (a) and (b) to form a Fab;
wherein the Fab binds to subdomain II of human HER2 and the Fv fragment binds to subdomain IV of human HER2, or wherein the Fab binds to subdomain IV of human HER2 and the Fv fragment binds to subdomain II of human HER2.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

In some embodiments of the bispecific protein, the first Fc polypeptide is fused to the V_{H} region of the Fv fragment and the second Fc polypeptide is fused to the V_{L} region of the Fv fragment. In some embodiments, the first Fc polypeptide is fused to the V_{L} region of the Fv fragment and the second Fc polypeptide is fused to the V_{H} region of the Fv fragment.

In some embodiments, the Fab that specifically binds to the subdomain II of human HER2 comprises a V_{H} region having at least 90% (e.g., 91%, 92%, 93%, 94%, 95%, 96%, 97%, 98%, 99%, or 100%) identity to the sequence of SEQ ID NO:108. In some embodiments, the Fab that specifically binds to the subdomain IV of human HER2 comprises a V_{H} region having at least 90% (e.g., 91%, 92%, 93%, 94%, 95%, 96%, 97%, 98%, 99%, or 100%) identity to the sequence of SEQ ID NO:109.

In some embodiments, a first linker connects the V_{H} region or the V_{L} region to the C-terminus of the Fc polypeptide. In some embodiments, the first linker or the second linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. Various linkers are described in detail further herein. In some embodiments, a first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, in part (a) of the bispecific protein having the structure “mAb/C-terminal V_{H} V_{L} on HC,” the first Fc polypeptide is fused at the N-terminus to the Fd portion of a Fab via a hinge region or a partial hinge region. In some embodiments, in part (b) of the bispecific protein having the structure “mAb/C-terminal V_{H} V_{L} on HC,” the second Fc polypeptide is fused at the N-terminus to the Fd portion of a Fab via a hinge region or a partial hinge region. An illustrative hinge region sequence is the human IgG1 hinge sequence EPKSCDKTHTCPPCP (SEQ ID NO:127). A partial hinge region refers to a portion of the sequence of SEQ ID NO:127, for example, a partial hinge region having the sequence of DKTHTCPPCP (SEQ ID NO:128).

In the bispecific protein having the structure “mAb/C-terminal V_{H} V_{L} on HC,” the first Fc polypeptide and/or the second Fc polypeptide can specifically bind to a transferrin receptor (e.g., a TfR-binding Fc polypeptide). Different Fc polypeptides and the modifications thereof are described in detail further herein. In some embodiments, the first Fc polypeptide and the second Fc polypeptide can each comprise modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In particular embodiments of the bispecific protein having the structure “mAb/N-terminal V_{H} V_{L} on HC,” the first Fc polypeptide (or the second Fc polypeptide) is a TfR-binding Fc polypeptide that comprises a T366W substitution, L234A and L235A substitutions (optionally including a P329G or a P329S substitution), and optionally a S239D and/or a I332E substitution, according to EU numbering, and the second Fc polypeptide (or the first Fc polypeptide) comprises T366S, L368A, and Y407V substitutions and optionally a S239D and/or a I332E substitution, according to EU numbering. For example, the first Fc polypeptide (or the second Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:137 and 186-196, and the second Fc polypeptide (or the first Fc polypeptide) can comprise a sequence having at least 90% (e.g., 91%, 92%, 93%, 94%, 95%, 96%, 97%, 98%, 99%, or 100%) identity to the sequence of any one of SEQ ID NOS:133 and 183-185).

In certain embodiments, the first Fc polypeptide is a TfR-binding Fc polypeptide and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain embodiments, the first Fc polypeptide does not include the L234A or L325A substitutions (or the P329G or P329S substitution if present in the second Fc polypeptide) and the second Fc polypeptide is a TfR-binding Fc polypeptide and contains L234A and L235A substitutions (optionally including a P329G or a P329S substitution), according to EU numbering.

Exemplary Bispecific “mAb/C-Terminal V_{H} V_{L} on HC” Proteins

In some embodiments, the bispecific protein comprises:
(a) a first Fc polypeptide that is fused at the N-terminus to an Fd portion of a Fab that binds to subdomain II of human HER2, and is fused at the C-terminus to a V_{H} region of an Fv fragment that binds to subdomain IV of human HER2;
(b) a second Fc polypeptide that is fused at the N-terminus to an Fd portion of the Fab, and is fused at the C-terminus to a V_{L} region of the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:82 or 83, (b) comprises a sequence having at least 90% (e.g., 91%, 92%, 93%, 94%, 95%, 96%, 97%, 98%, 99%, or 100%) identity to the sequence of SEQ ID NO:99, 100, or 101, and each of the two light chains in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:84, (b) comprises a sequence having at least 90% (e.g., 91%, 92%, 93%, 94%, 95%, 96%, 97%, 98%, 99%, or 100%) identity to the sequence of SEQ ID NO:98, and each of the two light chains in (c) comprises a sequence having at least 90% (e.g., 91%, 92%, 93%, 94%, 95%, 96%, 97%, 98%, 99%, or 100%) identity to the sequence of SEQ ID NO:25. In another example, (a) comprises a sequence having at least 90% (e.g., 91%, 92%, 93%, 94%, 95%, 96%, 97%, 98%, 99%, or 100%) identity to the sequence of SEQ ID NO:85, (b) comprises a sequence having at least 90% (e.g., 91%, 92%, 93%, 94%, 95%, 96%, 97%, 98%, 99%, or 100%) identity to the sequence of SEQ ID NO:97, and each of the two light chains in (c) comprises a sequence having at least 90% (e.g., 91%, 92%, 93%, 94%, 95%, 96%, 97%, 98%, 99%, or 100%) identity to the sequence of SEQ ID NO:25. In yet another example, (a) comprises a sequence having at least 90% (e.g., 91%, 92%, 93%, 94%, 95%, 96%, 97%, 98%, 99%, or 100%) identity to the sequence of SEQ ID NO:86, 87, or 88, (b) comprises a sequence having at least 90% (e.g., 91%, 92%, 93%, 94%, 95%, 96%, 97%, 98%, 99%, or 100%) identity to the sequence of SEQ ID NO:95 or 96, and each of the two light chains in (c) comprises a sequence having at least 90% (e.g., 91%, 92%, 93%, 94%, 95%, 96%, 97%, 98%, 99%, or 100%) identity to the sequence of SEQ ID NO:25.

In some embodiments, the bispecific protein comprises:
(a) a first Fc polypeptide that is fused at the N-terminus to an Fd portion of a Fab that binds to subdomain IV of human HER2, and is fused at the C-terminus to a V_{H} region of an Fv fragment that binds to subdomain II of human HER2;
(b) a second Fc polypeptide that is fused at the N-terminus to an Fd portion of the Fab, and is fused at the C-terminus to a V_{L} region of the Fv fragment.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution, according to EU numbering.

In one example of the bispecific protein, (a) comprises a sequence having at least 90% (e.g., 91%, 92%, 93%, 94%, 95%, 96%, 97%, 98%, 99%, or 100%) identity to the sequence of SEQ ID NO:89 or 90, (b) comprises a sequence having at least 90% (e.g., 91%, 92%, 93%, 94%, 95%, 96%, 97%, 98%, 99%, or 100%) identity to the sequence of SEQ ID NO:105, 106, or 107, and each of the two light chains in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:91, (b) comprises a sequence having at least 90% (e.g., 91%, 92%, 93%, 94%, 95%, 96%, 97%, 98%, 99%, or 100%) identity to the sequence of SEQ ID NO:104, and each of the two light chains in (c) comprises a sequence having at least 90% (e.g., 91%, 92%, 93%, 94%, 95%, 96%, 97%, 98%, 99%, or 100%) identity to the sequence of SEQ ID NO:26. In another example, (a) comprises a sequence having at least 90% (e.g., 91%, 92%, 93%, 94%, 95%, 96%, 97%, 98%, 99%, or 100%) identity to the sequence of SEQ ID NO:92, (b) comprises a sequence having at least 90% (e.g., 91%, 92%, 93%, 94%, 95%, 96%, 97%, 98%, 99%, or 100%) identity to the sequence of SEQ ID NO:103, and each of the two light chains in (c) comprises a sequence having at least 90% (e.g., 91%, 92%, 93%, 94%, 95%, 96%, 97%, 98%, 99%, or 100%) identity to the sequence of SEQ ID NO:26. In yet another example, (a) comprises a sequence having at least 90% (e.g., 91%, 92%, 93%, 94%, 95%, 96%, 97%, 98%, 99%, or 100%) identity to the sequence of SEQ ID NO:93 or 94, (b) comprises a sequence having at least 90% (e.g., 91%, 92%, 93%, 94%, 95%, 96%, 97%, 98%, 99%, or 100%) identity to the sequence of SEQ ID NO:102, and each of the two light chains in (c) comprises a sequence having at least 90% (e.g., 91%, 92%, 93%, 94%, 95%, 96%, 97%, 98%, 99%, or 100%) identity to the sequence of SEQ ID NO:26.

In some embodiments of the bispecific protein described above, the first Fc polypeptide comprises Leu at positions 234 and 235, according to EU numbering. In some embodiments, the second Fc polypeptide comprises Leu at positions 234 and 235, according to EU numbering. In certain embodiments, the protein includes the cis-LALA configuration described below.

In some embodiments of the bispecific protein described above, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the bispecific proteins.

### IV. Fc Polypeptides and Modifications Thereof

In some aspects, any of the anti-HER2 bispecific proteins described herein comprises an Fc polypeptide dimer in which either one or both Fc polypeptides in the dimer contain amino acid modifications relative to a wild-type Fc polypeptide. In some embodiments, the amino acid modifications in an Fc polypeptide (e.g., a modified Fc polypeptide) can result in binding of the Fc polypeptide dimer to a BBB receptor (e.g., a TfR), promote heterodimerization of the two Fc polypeptides in the dimer, modulate effector function, extend serum half-life, influence glycosylation, and/or reduce immunogenicity in humans. In some embodiments, the Fc polypeptides present in the bispecific protein independently have an amino acid sequence identity of at least about 85%, 90%, 95%, 96%, 97%, 98%, or 99% to a corresponding wild-type Fc polypeptide (e.g., a human IgG1, IgG2, IgG3, or IgG4 Fc polypeptide). Examples and descriptions of modified Fc polypeptides (e.g., TfR-binding Fc polypeptides) can be found, e.g., in International Patent Publication No. WO 2018/152326, which is incorporated herein by reference in its entirety.

### Fc Polypeptide Modifications for BBB Receptor Binding

Provided herein are anti-HER2 bispecific proteins that are capable of being transported across the BBB. Such a protein comprises a modified Fc polypeptide that binds to a BBB receptor. BBB receptors are expressed on BBB endothelia, as well as other cell and tissue types. In some embodiments, the BBB receptor is a TfR. A modified Fc polypeptide that binds to TfR is also referred to as having a TfR-binding site.

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein. In some embodiments, the domain that is modified for BBB (e.g., TfR) receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%).

In some embodiments, a BBB (e.g., TfR) receptor-binding Fc polypeptide present in a bispecific protein described herein comprises one or more at least one, two, or three substitutions; and in some embodiments, at least four, five, six, seven, eight, nine, or ten substitutions at amino acid positions comprising 266, 267, 268, 269, 270, 271, 295, 297, 298, and 299, according to the EU numbering scheme. In some embodiments, a BBB (e.g., TfR) receptor-binding Fc polypeptide present in a bispecific protein described herein comprises at least one, two, or three substitutions; and in some embodiments, at least four, five, six, seven, eight, or nine substitutions at amino acid positions comprising 274, 276, 283, 285, 286, 287, 288, 289, and 290, according to the EU numbering scheme. In some embodiments, a BBB (e.g., TfR) receptor-binding Fc polypeptide present in a bispecific protein described herein comprises at least one, two, or three substitutions; and in some embodiments, at least four, five, six, seven, eight, nine, or ten substitutions at amino acid positions comprising 268, 269, 270, 271, 272, 292, 293, 294, 296, and 300, according to the EU numbering scheme. In some embodiments, a BBB (e.g., TfR) receptor-binding Fc polypeptide present in a bispecific protein described herein comprises at least one, two, or three substitutions; and in some embodiments, at least four, five, six, seven, eight, or nine substitutions at amino acid positions comprising 272, 274, 276, 322, 324, 326, 329, 330, and 331, according to the EU numbering scheme. In some embodiments, a BBB (e.g., TfR) receptor-binding Fc polypeptide present in a bispecific protein described herein comprises at least one, two, or three substitutions; and in some embodiments, at least four, five, six, or seven substitutions at amino acid positions comprising 345, 346, 347, 349, 437, 438, 439, and 440, according to the EU numbering scheme.

In some embodiments, a BBB (e.g., TfR) receptor-binding Fc polypeptide present in a bispecific protein described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

In some embodiments, a BBB (e.g., TfR) receptor-binding Fc polypeptide comprises at least one position having a substitution, relative to SEQ ID NO:130, as follows: Leu, Tyr, Met, or Val at position 384; Leu, Thr, His, or Pro at position 386; Val, Pro, or an acidic amino acid at position 387; an aromatic amino acid, e.g., Trp or Gly (e.g., Trp) at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, a BBB (e.g., TfR) receptor-binding Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four five, six, seven, or all eight of positions 384, 386, 387, 388, 389, 413, 416, and 421 have an amino acid substitution as specified in this paragraph.

In some embodiments, a Fc polypeptide having modifications in amino acid positions 384, 386, 387, 388, 389, 390, 413, 416, and/or 421 comprises a native Asn at position 390. In some embodiments, the Fc polypeptide comprises Gly, His, Gln, Leu, Lys, Val, Phe, Ser, Ala, or Asp at position 390. In some embodiments, the Fc polypeptide further comprises one, two, three, or four substitutions at positions comprising 380, 391, 392, and 415. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the Fc polypeptide comprises two, three, four, five, six, seven, eight nine, or ten positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the BBB (e.g., TfR) receptor-binding Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the Fc polypeptide further comprises Ser, Thr, Gln, or Phe at position 391. In some embodiments, the Fc polypeptide further comprises Trp, Tyr, Leu, or Gln at position 380 and/or Gln, Phe, or His at position 392. In some embodiments, Trp is present at position 380 and/or Gln is present at position 392. In some embodiments, a BBB (e.g., TfR) receptor-binding Fc polypeptide does not have a Trp at position 380.

In other embodiments, a BBB (e.g., TfR) receptor-binding Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the BBB (e.g., TfR) receptor-binding Fc polypeptide comprises a native Asn at position 390. In certain embodiments, the Fc polypeptide further comprises Trp, Tyr, Leu, or Gln at position 380; and/or Glu at position 415. In some embodiments, the Fc polypeptide further comprises Trp at position 380 and/or Glu at position 415.

In some embodiments, the BBB (e.g., TfR) receptor-binding Fc polypeptide comprises one or more of the following substitutions: Trp at position 380; Thr at position 386; Trp at position 388; Val at position 389; Ser or Thr at position 413; Glu at position 415; and/or Phe at position 421.

In additional embodiments, the BBB (e.g., TfR) receptor-binding Fc polypeptide further comprises one, two, or three positions selected from the following: position 414 is Lys, Arg, Gly, or Pro; position 424 is Ser, Thr, Glu, or Lys; and position 426 is Ser, Trp, or Gly.

In some embodiments, the BBB (e.g., TfR) receptor-binding Fc polypeptide has the sequence of SEQ ID NO:135. In some embodiments of the bispecific proteins described herein, one of the two Fc polypeptides in the Fc polypeptide dimer can be a BBB (e.g., TfR) receptor-binding Fc polypeptide having the sequence of SEQ ID NO:135, while the other Fc polypeptide in the Fc polypeptide dimer can have the sequence of a wild-type Fc polypeptide (e.g., SEQ ID NO:130). In other embodiments of the bispecific proteins described herein, both Fc polypeptides in the Fc polypeptide dimer can be a BBB (e.g., TfR) receptor-binding Fc polypeptide having the sequence of SEQ ID NO:135.

In some embodiments of the bispecific proteins described herein, the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:135-139 and 186-196.

In some embodiments of the bispecific proteins described herein, one of the two Fc polypeptides in the Fc polypeptide dimer can be a BBB (e.g., TfR) receptor-binding Fc polypeptide comprising Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:135, while the other Fc polypeptide in the Fc polypeptide dimer can have the sequence of a wild-type Fc polypeptide (e.g., SEQ ID NO:130).

In some embodiments of the bispecific proteins described herein, the first Fc polypeptide and/or the second Fc polypeptide independently comprises Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:140-144.

In some embodiments of the bispecific proteins described herein, one of the two Fc polypeptides in the Fc polypeptide dimer can be a BBB (e.g., TfR) receptor-binding Fc polypeptide comprising Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:140, while the other Fc polypeptide in the Fc polypeptide dimer can have the sequence of a wild-type Fc polypeptide (e.g., SEQ ID NO:130).

In some embodiments, the BBB (e.g., TfR) receptor-binding Fc polypeptide has the sequence of SEQ ID NO:140. In some embodiments of the bispecific proteins described herein, one of the two Fc polypeptides in the Fc polypeptide dimer can be a BBB (e.g., TfR) receptor-binding Fc polypeptide having the sequence of SEQ ID NO:140, while the other Fc polypeptide in the Fc polypeptide dimer can have the sequence of a wild-type Fc polypeptide (e.g., SEQ ID NO:130). In other embodiments of the bispecific proteins described herein, both Fc polypeptides in the Fc polypeptide dimer can be a BBB (e.g., TfR) receptor-binding Fc polypeptide having the sequence of SEQ ID NO:140.

In some embodiments, the BBB (e.g., TfR) receptor-binding Fc polypeptide has the sequence of SEQ ID NO:145. In some embodiments of the bispecific proteins described herein, one of the two Fc polypeptides in the Fc polypeptide dimer can be a BBB (e.g., TfR) receptor-binding Fc polypeptide having the sequence of SEQ ID NO:145, while the other Fc polypeptide in the Fc polypeptide dimer can have the sequence of a wild-type Fc polypeptide (e.g., SEQ ID NO:130). In other embodiments of the bispecific proteins described herein, both Fc polypeptides in the Fc polypeptide dimer can be a BBB (e.g., TfR) receptor-binding Fc polypeptide having the sequence of SEQ ID NO:145.

### Fc Polypeptide Modifications for Heterodimerization

In some embodiments, the Fc polypeptides present in any bispecific protein described herein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the bispecific protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a bispecific protein described herein has the T366W knob mutation and the other Fc polypeptide has the Y407V mutation, which is typically accompanied by the T366S and L368A hole mutations.

In some embodiments, one or both Fc polypeptides present in a bispecific protein described herein may also be engineered to contain other modifications for heterodimerization, e.g., electrostatic engineering of contact residues within a CH3-CH3 interface that are naturally charged or hydrophobic patch modifications.

For example, in some embodiments, a bispecific protein described herein can contain an Fc polypeptide dimer that has one Fc polypeptide having the T366W knob mutation and at least 90% (e.g., 91%, 92%, 93%, 94%, 95%, 96%, 97%, 98%, or 99%) identity to the sequence of SEQ ID NO:131 and the other Fc polypeptide having the T366S, L368A, and Y407V hole mutations and at least 90% identity to the sequence of SEQ ID NO:133. In certain embodiments, one or both Fc polypeptides in the Fc polypeptide dimer can be a TfR-binding Fc polypeptide. In particular embodiments, a bispecific protein described herein can contain an Fc polypeptide dimer that has (i) a first Fc polypeptide having the sequence of SEQ ID NO:133, and (ii) a second Fc polypeptide having the sequence of SEQ ID NO:136. In particular embodiments, a bispecific protein described herein can contain an Fc polypeptide dimer that has (i) a first Fc polypeptide having the sequence of SEQ ID NO:133, and (ii) a second Fc polypeptide having the sequence of SEQ ID NO:141. In particular embodiments, a bispecific protein described herein can contain an Fc polypeptide dimer that has (i) a first Fc polypeptide having at least 90% identity to the sequence of SEQ ID NO:133, and (ii) a second Fc polypeptide having the sequence of SEQ ID NO:146.

### Fc Polypeptide Modifications for Modulating Effector Function

In some embodiments, one or both Fc polypeptides present in any bispecific protein described herein may comprise modifications that reduce TfR-mediated effector function upon TfR binding, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a bispecific protein described herein may comprise modifications that reduce or eliminate TfR-mediated effector function. Illustrative Fc polypeptide mutations that reduce TfR-mediated effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (also referred to as “LALA” herein) substitutions.

Additional Fc polypeptide mutations that modulate an effector function include, but are not limited to, the following: position 329 may have a mutation in which proline is substituted with a glycine, alanine, serine, or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme. In some embodiments, one or both Fc polypeptides may have L234A, L235A, and P329G or P329S substitutions, according to the EU numbering scheme.

In some embodiments, one or both Fc polypeptides present in a bispecific protein described herein may comprise modifications that are capable of enhancing HER2-mediated effector function upon HER2 binding, i.e., enhancing the ability to induce certain biological functions upon binding to an Fc receptor expressed on an effector cell that mediates the effector function. Examples of antibody effector functions are described above. Illustrative Fc polypeptide mutations that are capable of enhancing HER2-mediated effector function include, but are not limited to, substitutions in a CH2 domain, e.g., at positions 239 and/or 332, according to the EU numbering scheme. For example, in some embodiments, one or both Fc polypeptides can comprise aspartic acid at position 239 and/or glutamic acid at position 332. Thus, one or both Fc polypeptides may have a S239D and/or a I332E substitution, according to EU numbering.

“cis-LALA” Configuration

In some embodiments of any bispecific protein described herein, only one of the two Fc polypeptides (but not both Fc polypeptides) of the two Fc polypeptides in the bispecific protein is modified to reduce TfR-mediated effector function upon TfR binding. The other Fc polypeptide does not contain a TfR-binding site or any modifications that reduce effector function. The Fc polypeptide dimer in the bispecific protein that has only one of the two Fc polypeptides containing both the TfR-binding site and modifications that reduce FcγR binding (e.g., LALA substitutions) when bound to TfR, while the other Fc polypeptide does not contain a TfR-binding site or any modifications that reduce FcγR binding, is referred to as having the cis-LALA configuration.

For example, in some embodiments, a bispecific protein described herein can contain an Fc polypeptide dimer having the cis-LALA configuration that has (i) a first Fc polypeptide having the sequence of SEQ ID NO:137, which has both a TfR-binding site and LALA substitutions, as well as a knob modification, and (ii) a second Fc polypeptide having at least 90% identity to the sequence of SEQ ID NO:133, which only has a hole modification. In some embodiments, a bispecific protein described herein can contain an Fc polypeptide dimer having the cis-LALA configuration that has (i) a first Fc polypeptide having the sequence of SEQ ID NO:142, which has both a TfR-binding site and LALA substitutions, as well as a knob modification, and (ii) a second Fc polypeptide having at least 90% identity to the sequence of SEQ ID NO:133, which only has a hole modification. In some embodiments, a bispecific protein described herein can contain an Fc polypeptide dimer having the cis-LALA configuration that has (i) a first Fc polypeptide having the sequence of SEQ ID NO:147, which has both a TfR-binding site and LALA substitutions, as well as a knob modification, and (ii) a second Fc polypeptide having at least 90% identity to the sequence of SEQ ID NO:133, which only has a hole modification.

In particular embodiments, a bispecific protein described herein can contain an Fc polypeptide dimer having the cis-LALA configuration that has (i) a first Fc polypeptide comprising Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137, and (ii) a second Fc polypeptide comprising Ser at position 366, Ala at position 368, and Val at position 407, according to EU numbering, and a sequence having at least 90% identity to the sequence of SEQ ID NO:133.

In particular embodiments, a bispecific protein described herein can contain an Fc polypeptide dimer having the cis-LALA configuration that has (i) a first Fc polypeptide comprises Ser at position 366, Ala at position 368, and Val at position 407, according to EU numbering, and a sequence having at least 90% identity to the sequence of SEQ ID NO:133, and (ii) a second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137.

In particular embodiments, a bispecific protein described herein can contain an Fc polypeptide dimer having the cis-LALA configuration that has (i) a first Fc polypeptide comprising Ala at position 234, Ala at position 235, Trp at position 366, Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:142, and (ii) a second Fc polypeptide comprising Ser at position 366, Ala at position 368, and Val at position 407, according to EU numbering, and a sequence having at least 90% identity to the sequence of SEQ ID NO:133.

In particular embodiments, a bispecific protein described herein can contain an Fc polypeptide dimer having the cis-LALA configuration that has (i) a first Fc polypeptide comprising Ser at position 366, Ala at position 368, and Val at position 407, according to EU numbering, and a sequence having at least 90% identity to the sequence of SEQ ID NO:133, and (ii) a second Fc polypeptide comprising Ala at position 234, Ala at position 235, Trp at position 366, Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:142.

### Fc Polypeptide Modifications for Extending Serum Half-Life

In some embodiments, modifications to enhance serum half-life may be introduced into any bispecific protein described herein. For example, in some embodiments, one or both Fc polypeptides present in a bispecific protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

Fc Polypeptide with C-Terminal Lysine Residue Removed

In some embodiments of the bispecific proteins described herein, one or both of the Fc polypeptides can have its C-terminal lysine removed (e.g., the Lys residue at position 447 of the Fc polypeptide, according to EU numbering). The C-terminal lysine residue is highly conserved in immunoglobulins across many species and may be fully or partially removed by the cellular machinery during protein production. In some embodiments, removal of the C-terminal lysines in the Fc polypeptides can improve the stability of the bispecific proteins.

### V. Linkers

As described herein, a bispecific protein can contain one or more linkers. A linker refers to a linkage between two elements (i.e., between a V_{H} region and a V_{L} region, between an scFv and an Fc polypeptide, between an scFv and an Fd portion, between an scFv and a light chain, between a V_{H} region or a V_{L} region and an Fd portion, between a V_{H} region or a V_{L} region and a light chain, or between a V_{H} region or a V_{L} region and an Fc polypeptide) in the bispecific protein. In some embodiments, a linker can be a peptide linker that can link two elements in the bispecific protein to provide space and/or flexibility.

In some embodiments, a linker can include 1-100 amino acids (e.g., 1-90, 1-80, 1-70, 1-90, 1-60, 1-50, 1-40, 1-30, 1-20, 1-10, 1-8, 1-6, 1-4, 5-100, 10-100, 15-100, 20-100, 30-100, 40-100, 50-100, 60-100, 70-100, 80-100, 90-100, 10-90, 10-80, 10-70, 10-60, 10-50, 10-40, 10-30, 10-25, 10-20, or 10-15 amino acids). In some embodiments, a linker between two Fc domain monomers is an amino acid spacer containing 1-30 amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids). Linkers can contain natural amino acids, unnatural amino acids, or a combination thereof. In some embodiments, the linker can be a flexible linker, e.g., containing amino acids such as Gly, Asn, Ser, Thr, Ala, and the like. Such linkers can be designed using known parameters and may be of any length and contain any number of repeat units of any length (e.g., repeat units of Gly and Ser residues). For example, the linker may have repeats, such as two, three, four, five, or more GGGGS (SEQ ID NO:117), GGSG (SEQ ID NO:151), GSGG (SEQ ID NO:152), or SGGG (SEQ ID NO:153) repeats or a single GGGGS (SEQ ID NO:117), GGSG (SEQ ID NO:151), GSGG (SEQ ID NO:152), or SGGG (SEQ ID NO:153). Examples of flexible linkers containing Gly and Ser residues include, but are not limited to, GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

In some embodiments, a linker can also contain amino acids other than glycine and serine, e.g., GGGGSEPKSS (SEQ ID NO:124), ASTKGPSVF (SEQ ID NO:125), or RTVAAPSVFI (SEQ ID NO:126). Example of other linkers are also described in the art, see, e.g., Chen et al. Adv. Drug Deliv Rev. 65(10):1357-1369, 2013.

### VI. Preparation of Bispecific Proteins

For preparing a bispecific protein described herein, many techniques known in the art can be used. In some embodiments, the genes encoding the heavy and light chains of an antibody of interest can be cloned from a cell, e.g, from a hybridoma. Gene libraries encoding heavy and light chains of monoclonal antibodies can also be made from hybridoma or plasma cells. Alternatively, phage or yeast display technology can be used to identify antibodies and Fab fragments that specifically bind to selected antigens.

Bispecific proteins can be produced using any number of expression systems, including prokaryotic and eukaryotic expression systems. In some embodiments, the expression system is a mammalian cell expression system, such as a hybridoma, or a CHO cell expression system. Many such systems are widely available from commercial suppliers. In some embodiments, the polynucleotides encoding the polypeptides that comprise the bispecific protein may be expressed using a single vector, e.g., in a di-cistronic expression unit, or under the control of different promoters. In other embodiments, the polynucleotides encoding the polypeptides that comprise the bispecific protein may be expressed using separate vectors.

In some aspects, the disclosure provides isolated nucleic acids comprising a nucleic acid sequence encoding any of the polypeptides comprising bispecific proteins as described herein; vectors comprising such nucleic acids; and host cells into which the nucleic acids are introduced that are used to replicate the nucleic acids and/or to express the bispecific proteins.

In some embodiments, a polynucleotide (e.g., an isolated polynucleotide) comprises a nucleotide sequence encoding a polypeptide that comprises the bispecific protein as disclosed herein (e.g., as described in Section III above). In some embodiments, the polynucleotide comprises a nucleotide sequence encoding one or more amino acid sequences (e.g., heavy chain, light chain, and/or Fc polypeptide sequences) disclosed in the Informal Sequence Listing below. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence disclosed in the Informal Sequence Listing below. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making a bispecific protein as described herein are provided. In some embodiments, the method includes culturing a host cell as described herein (e.g., a host cell expressing a polynucleotide or vector as described herein) under conditions suitable for expression of the bispecific protein. In some embodiments, the bispecific protein is subsequently recovered from the host cell (or host cell culture medium). In some embodiments, the bispecific protein is purified, e.g., by chromatography.

### VII. Therapeutic Methods

In some aspects, provided herein are methods for treating a cancer (e.g., a HER2-positive cancer) or treating brain metastasis of a cancer (e.g., a HER2-positive cancer) in a subject by administering to the subject a therapeutically effective amount of any bispecific protein described herein or a pharmaceutical composition containing thereof. Also provided herein are methods of transcytosis of an antibody variable region that is capable of binding HER2 (e.g., human HER2), or an antigen-binding fragment thereof, across an endothelium. In some embodiments, the methods comprise contacting the endothelium with a composition comprising a bispecific protein described herein. In some embodiments, the endothelium is the blood brain barrier (BBB).

Non-limiting examples of HER2-positive cancers that can be treated according to the methods provided herein include HER2-positive breast, ovarian, bladder, salivary gland, endometrial, pancreatic, and non-small-cell lung cancer (NSCLC), as well as HER2-positive gastric adenocarcinoma and/or a HER2-positive gastroesophageal junction adnocarcinoma. In some embodiments, the HER2-positive cancer is a HER2-positive breast cancer. In some embodiments, the HER2-positive cancer is a HER2-positive gastric adenocarcinoma and/or a HER2-positive gastroesophageal junction adnocarcinoma. In some embodiments, the HER2-positive cancer is a metastatic cancer.

In still other aspects, provided herein are methods for treating metastasis of a cancer (e.g., a HER2-positive cancer). In some embodiments, the methods comprise administering to the subject a therapeutically effective amount of an anti-HER2 bispecific protein described herein. In some embodiments, the metastasis is a brain metastasis of a HER2-positive cancer described above. In some embodiments, the metastasis is a brain metastasis of a HER2-positive breast cancer. In some embodiments, the metastasis is a brain metastasis of a HER2-positive gastric adenocarcinoma and/or a HER2-positive gastroesophageal junction adnocarcinoma.

In some embodiments, the therapeutic benefit can comprise a decrease in or slowing of tumor growth, a decrease in tumor size (e.g., volume), a decrease in tumor cell viability, a decrease in the number of metastatic lesions, amelioration in one or more signs or symptoms of a cancer (e.g., HER2-positive cancer), and/or an increase in patient survival. In some embodiments, tumor cell survival, tumor growth, tumor size, and/or the number of metastatic lesions is decreased by at least about 10%, 20%, 30%, 40%, 50%, 60%, 70%, 80%, 90%, or more.

In some embodiments, the anti-HER2 bispecific protein antagonizes HER2 activity. In some embodiments, HER2 activity is inhibited (e.g., by at least about 10%, 20%, 30%, 40%, 50%, 60%, 70%, 80%, 90%, or more).

The route of administration of an anti-HER2 bispecific protein described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the anti-HER2 bispecific protein is administered orally, intravenously, or intraperitoneally.

### VIII. Pharmaceutical Compositions and Kits

In other aspects, pharmaceutical compositions and kits comprising a anti-HER2 bispecific protein in accordance with the disclosure are provided.

### Pharmaceutical Compositions

Guidance for preparing formulations for use in the disclosure can be found in any number of handbooks for pharmaceutical preparation and formulation that are known to those of skill in the art.

In some embodiments, a pharmaceutical composition comprises an anti-HER2 bispecific protein as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

In some embodiments, the bispecific protein can be formulated for parenteral administration by injection. Typically, a pharmaceutical composition for use in in vivo administration is sterile, e.g., heat sterilization, steam sterilization, sterile filtration, or irradiation.

Dosages and desired drug concentration of pharmaceutical compositions described herein may vary depending on the particular use envisioned.

### Kits

In some embodiments, a kit for use in treating a cancer (e.g., a HER2-positive cancer) comprising a bispecific protein described herein is provided. In some embodiments, the kit further comprises one or more additional therapeutic agents. For example, in some embodiments, the kit comprises a bispecific protein as described herein and further comprises one or more additional therapeutic agents for use in the treatment of cancer. In some embodiments, the kit further comprises instructional materials containing directions (i.e., protocols) for the practice of the methods described herein (e.g., instructions for using the kit for administering a bispecific protein). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### IX. Examples

The present invention will be described in greater detail by way of specific examples. The following examples are offered for illustrative purposes only, and are not intended to limit the invention in any manner.

### Example 1. Generation of Bispecific Proteins

Engineered proteins having the structure of a bispecific protein as described herein and shown in FIGS. 1-5 were generated to target subdomain II of human HER2 and subdomain IV of human HER2. In some constructs, the C-terminal lysine of the Fc polypeptide was removed. In some constructs, the Fd portions (V_{H}+CH1) were cloned into expression vectors comprising a sequence encoding an Fc polypeptide. In some embodiments, the Fc polypeptide was engineered to have a TfR-binding site. In some embodiments of the constructs, one of the Fc polypeptides contained a TfR-binding site and a “knob” (T366W) mutation (e.g., SEQ ID NO:137), while the other Fc polypeptide contained “hole” (T366S/L368A/Y407V) mutations (e.g., SEQ ID NO:133). Additionally, one or both Fc polypeptides also contained mutations L234A/L235A, which attenuate FcγR binding.

Vectors were co-transfected to ExpiCHO or Expi293 cells along with the corresponding light chain vector in the ratio knob:hole:light chain of 1:1:2. The expressed protein was purified from conditioned media by loading the supernatant over a Protein A column. The column was washed with 10 column volumes of PBS, pH 7.4. The proteins were eluted with 50 mM sodium citrate, pH 3.0 containing 150 mM NaCl, and immediately neutralized with 200 mM arginine, 137 mM succinic acid, pH 5.0. The proteins were further purified by size-exclusion chromatography (SEC) (GE Superdex200) using 200 mM arginine, 137 mM succinic acid, pH 5.0 as running buffer. The purified proteins were confirmed by intact mass LC/MS, and purity of >95% was confirmed by SDS-PAGE and analytical HPLC-SEC. Anti-HER2 bispecific proteins containing unmodified human IgG1 constant regions were generated. Table 1 below provides the sequences of the anti-HER2 bispecific proteins and two control anti-HER2 antibodies.

TABLE 1

| | Heavy Chain 1 | Heavy Chain 2 | Light Chain |
|---|---|---|---|
| Bispecific Protein #1 | SEQ ID NO: 6 | SEQ ID NO: 10 | SEQ ID NO: 26 |
| Bispecific Protein #2 | SEQ ID NO: 1 | SEQ ID NO: 22 | SEQ ID NO: 25 |
| Bispecific Protein #3 | SEQ ID NO: 18 | SEQ ID NO: 5 | SEQ ID NO: 25 |
| Bispecific Protein #4 | SEQ ID NO: 6 | SEQ ID NO: 37 | SEQ ID NO: 26 |
| Bispecific Protein #5 | SEQ ID NO: 33 | SEQ ID NO: 38 | SEQ ID NO: 26 |
| Bispecific Protein #6 | SEQ ID NO: 63 | SEQ ID NO: 66 | SEQ ID NO: 79 |
| Bispecific Protein #7 | SEQ ID NO: 82 | SEQ ID NO: 99 | SEQ ID NO: 25 |
| Bispecific Protein #8 | SEQ ID NO: 90 | SEQ ID NO: 107 | SEQ ID NO: 26 |
| Bispecific Protein #9 | SEQ ID NO: 1 | SEQ ID NO: 44 | SEQ ID NO: 25 |
| Bispecific Protein #10 | SEQ ID NO: 6 | SEQ ID NO: 50 | SEQ ID NO: 26 |
| Bispecific Protein #11 | SEQ ID NO: 6 | SEQ ID NO: 156 | SEQ ID NO: 26 |
| Bispecific Protein #12 | SEQ ID NO: 45 | SEQ ID NO: 50 | SEQ ID NO: 26 |
| Bispecific Protein #13 | SEQ ID NO: 46 | SEQ ID NO: 156 | SEQ ID NO: 26 |
| Bispecific Protein #14 | SEQ ID NO: 6 | SEQ ID NO: 157 | SEQ ID NO: 26 |
| Bispecific Protein #15 | SEQ ID NO: 63 | SEQ ID NO: 67 | SEQ ID NO: 79 |
| Bispecific Protein #16 | SEQ ID NO: 82 | SEQ ID NO: 100 | SEQ ID NO: 25 |
| Bispecific Protein #17 | SEQ ID NO: 90 | SEQ ID NO: 158 | SEQ ID NO: 26 |
| Bispecific Protein #18 | SEQ ID NO: 1 | SEQ ID NO: 23 | SEQ ID NO: 25 |
| Bispecific Protein #19 | SEQ ID NO: 24 | SEQ ID NO: 5 | SEQ ID NO: 25 |
| Anti-HER2DIV control | SEQ ID NO: 154 | SEQ ID NO: 154 | SEQ ID NO: 26 |
| Anti-HER2DII control | SEQ ID NO: 155 | SEQ ID NO: 155 | SEQ ID NO: 25 |

### Example 2. Biacore Assessment of Bispecific Proteins

Affinities of the bispecific proteins were measured by SPR using a Biacore T200 or a Biacore 8K. Biacore™ Series S CM5 sensor chips were immobilized with monoclonal mouse anti-human IgG (Fc) antibody for HER2 affinity measurements or mouse anti-human Fab for TfR affinity measurements (human antibody or Fab capture kit from GE Healthcare). Serial 3-fold dilutions of analyte (recombinant HER2 extracellular domain or recombinant TfR apical domain) were injected at a flow rate of 30 L/min. Each sample was analyzed using a 3-minute association and a 10-minute dissociation for HER2 extracellular domain binding and a 40-second association and a 3-minute dissociation for human TfR apical domain binding. After each injection, the chip was regenerated using 3 M MgCl₂ or 50 mM glycine at pH 2.0. Binding response was corrected by subtracting the RU from a flow cell capturing an irrelevant IgG at similar density. A 1:1 Languir model of simultaneous fitting of kon and koff was used for kinetics analysis. The K_{D} for construct “anti-HER2_DIV/DII_scFv_1” is 2.3 nM and the K_{D} for construct “anti-HER2_DIV/DII_scFv_2” is 1.9 nM. The two constructs are described below.

Both constructs “anti-HER2_DIV/DII_scFv_1” and “anti-HER2_DIV/DII_scFv_2” have the “Fab-Fc polypeptide/scFv-Fc polypeptide” structure. In the construct “anti-HER2_DIV/DII_scFv_1,” (a) comprises the sequence of SEQ ID NO:1 (Anti-HER2DII Fab fused to the N-terminus of CH3C.35.23.4 with knob LALA via the hinge region), (b) comprises the sequence of SEQ ID NO:23 (Anti-HER2DIV scFv fused to the N-terminus of Fc polypeptide with hole mutation via a hinge region), and (c) comprises the sequence of SEQ ID NO:25. In the construct “anti-HER2_DIV/DII_scFv_2,” (a) comprises the sequence of SEQ ID NO:5 (Anti-HER2DII Fab fused to the N-terminus of Fe polypeptide with hole mutation via a hinge region), (b) comprises the sequence of SEQ TD NO:24 (Anti-HER2DIV scFv fused to the N-terminus of CH3C.35.23.4 with knob LALA via the hinge region), and (c) comprises the sequence of SEQ ID NO:25. In both constructs, the scFv portion contains an anti-TER2DIV V_{L} region having a Gin to Cys substitution at position 100 (SEQ ID NO:115) and an anti-HER2DIV V_{H} region having a Gly to Cys substitution at position 44 (SEQ ID NO:113). Also in both constructs, the hinge region in (b) has a Cys to Ser mutation at position 5 (EPKSSDKTHTCPPCP (SEQ ID NO: 129)).

Table 2 below further shows the K_{D} for HER2 binding and K_{D} for TfR binding of anti-HER2 bispecific proteins.

TABLE 2

| | HER2 binding | TfR binding |
|---|---|---|
| | K_{D} (nM) | K_{D} (nM) |
| Anti-HER2DIV control | 3.0 | NA |
| Anti-HER2DII control | 2.9 | NA |
| Bispecific Protein #1 | 3.0 | 460 |
| Bispecific Protein #2 | 0.024 | 480 |
| Bispecific Protein #3 | 0.027 | 450 |
| Bispecific Protein #4 | 0.019 | 470 |
| Bispecific Protein #5 | 0.031 | 540 |
| Bispecific Protein #6 | 0.0074 | 490 |
| Bispecific Protein #7 | 0.0038 | 350 |
| Bispecific Protein #8 | 0.0031 | 290 |
| Bispecific Protein #9 | 0.0033 | 500 |
| Bispecific Protein #10 | 0.0017 | 520 |
| Bispecific Protein #11 | 0.0028 | 470 |
| Bispecific Protein #12 | 0.0006 | 560 |
| Bispecific Protein #13 | 0.0008 | 600 |

### Example 3. Co-Targeting TfR and HER2 Subdomains II and IV

Many tumor cells and tumor cell lines, such as BT474 and OE19, express both HER2 and TfR. While it is well established that antibodies targeting HER2 subdomain IV are capable of inhibiting tumor cell growth and reducing tumor cell viability in some HER2⁺ cell lines, we sought to understand whether co-targeting HER2 subdomain IV and TfR would lead to enhanced cell killing.

Two constructs having the “Fab-Fc polypeptide/scFv-Fc polypeptide” structure were used. The construct “anti-HER2_DIV/DII_scFv_1” and the construct “anti-HER2_DIV/DII_scFv_2” are described in the previous example.

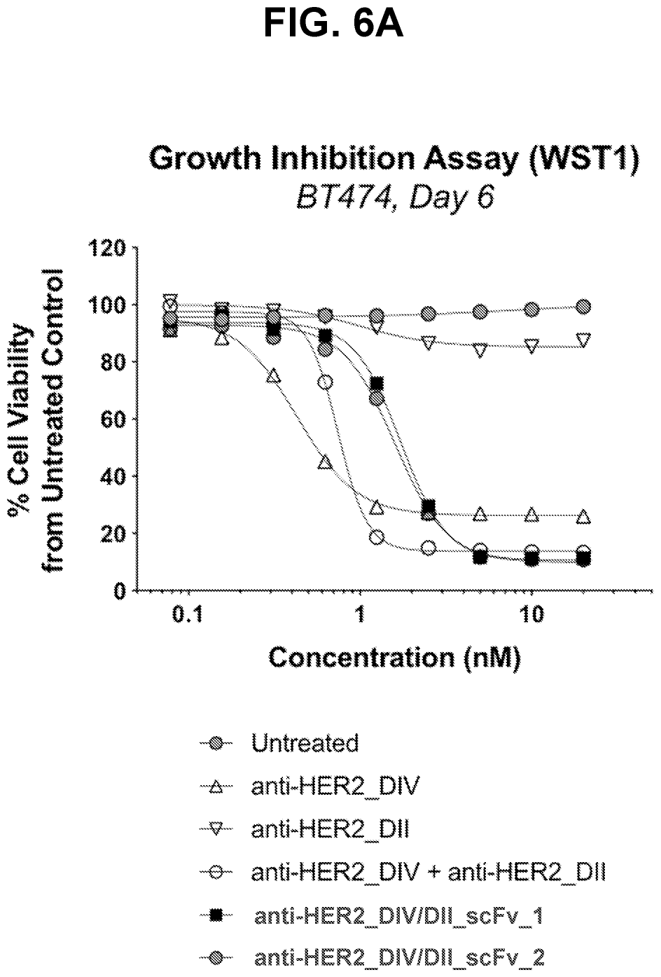

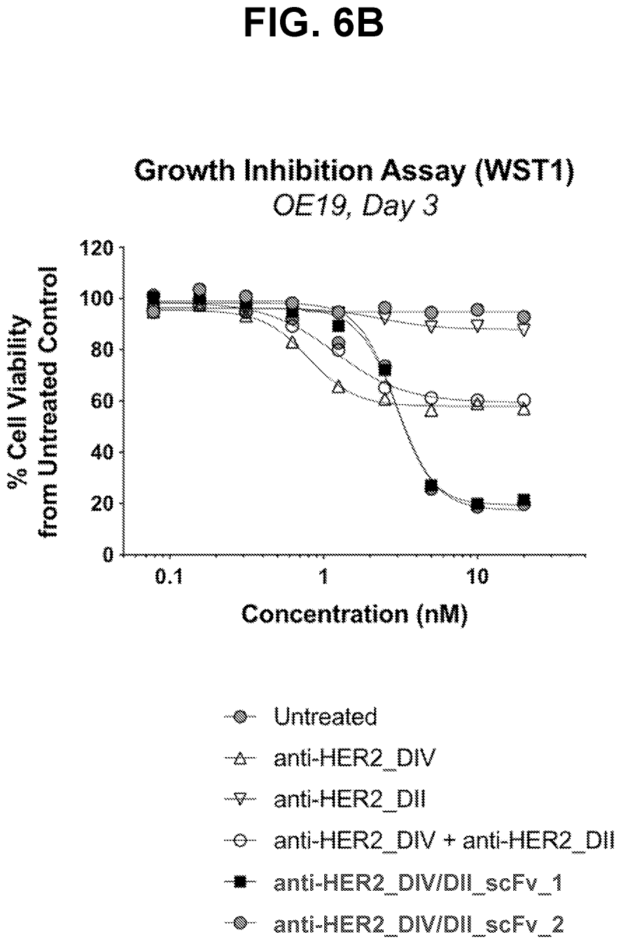

We first compared both constructs and control monoclonal anti-HER2 antibodies in a growth inhibition assay with a HER2⁺ tumor cell line BT474, which is sensitive to anti-HER2 therapies. BT474 cells were plated overnight at 10,000 cells/well in a 96-well plate, treated with 60 μL of 1:3 serial dilution of molecules of interest beginning at 166 nM (25,000 ng/mL). Culture media (RPMI) and drugs were replenished on Day 3. On Day 6, cell growth was determined using 5 μL of WST-1 reagent (Sigma Aldrich) in 50 μL of growth media. The plate was incubated for 4 hours in the presence of WST-1 reagent, and absorbance was determined at 440 nm. The percent of growth inhibition/proliferation was calculated based on A440 nM and was normalized to the untreated control. As shown in FIG. 6A, combination of anti-HER2-DIV and anti-HER2-DII reduced BT474 cell viability relative to the control with a maximum inhibition of about 87%. Similarly, anti-HER2_DIV/DII_scFv_1 and anti-HER2_DIV/DII_scFv_2 showed similar maximum growth inhibition compared to anti-HER2-DIV and anti-HER2-DII (FIG. 6A).

Next, we compared the two constructs and control monoclonal anti-HER2 antibodies with another HER2⁺ cancer cell line OE19 that is resistant to anti-HER2 treatment. As shown in FIG. 6B, unlike BT474, in which there was no difference between the maximum growth inhibition effects of the two anti-HER2_DIV/DII_scFv constructs and combination of anti-HER2-DIV and anti-HER2-DII, OE19 cell line had a maximum inhibition of about 80% upon anti-HER2_DIV/DII_scFv_1 and anti-HER2_DIV/DII_scFv_2 treatments while the cells were minimally responsive to combination of anti-HER2-DIV and anti-HER2-DII. These results indicate that targeting HER2 subdomain IV, HER2 subdomain II, and TfR could result in enhanced cell growth inhibition in an anti-HER2-resistant cell line as compared to targeting only the HER2 subdomains. Of note, any enhancement could be masked if the cell line is already sensitive to anti-HER2 therapy.

### Example 4. In Vivo Pharmacokinetic Properties of Anti-HER2 Bispecific Proteins

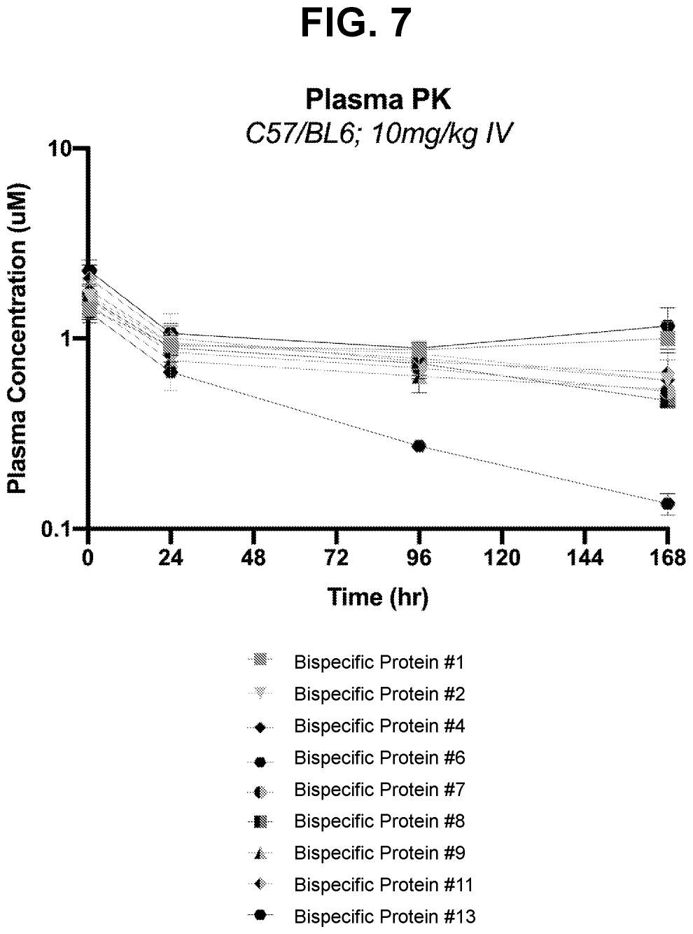

To evaluate the in vivo plasma pharmacokinetics of anti-HER2 bispecific proteins in the absence of TfR binding, C57BL/6 mice were intravenously administered 10 mg/kg of anti-HER2 bispecific proteins. Blood was collected at 30 min, 1 d, 4 d, and 7 d following a single dose, processed to plasma, and stored at −80° C. until analysis. The total therapeutic concentrations of the bispecific proteins in the plasma were quantified using an anti-human IgG sandwich ELISA (FIG. 7) and using the appropriate dosing solutions as a standard. Plates were coated overnight with an anti-human IgG then washed 3× with wash buffer. Standards and relevantly diluted samples were incubated with agitation for 2 hrs at room temperature. After incubation, plates were washed 3× with wash buffer. The detection antibody was diluted in blocking buffer and plates were incubated with agitation for 1 hr at room temperature. After a final 3× wash, plates were developed by adding TMB substrate and incubated for 5-10 minutes. Reaction was quenched and read using 450 nM absorbance (Biotek plate reader). All anti-HER2 bispecific proteins showed clearance values within the range of normal IgGs.

### Example 5. In Vivo Brain Uptake of Anti-HER2 Bispecific Proteins

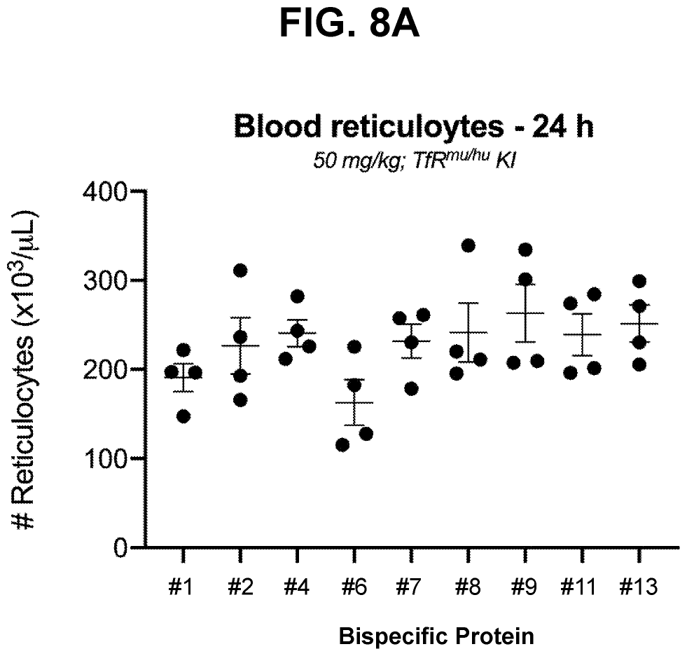

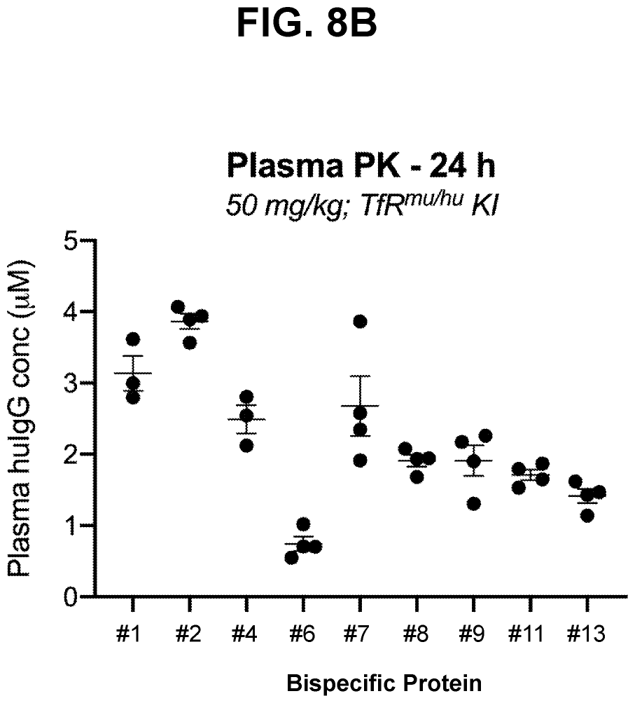

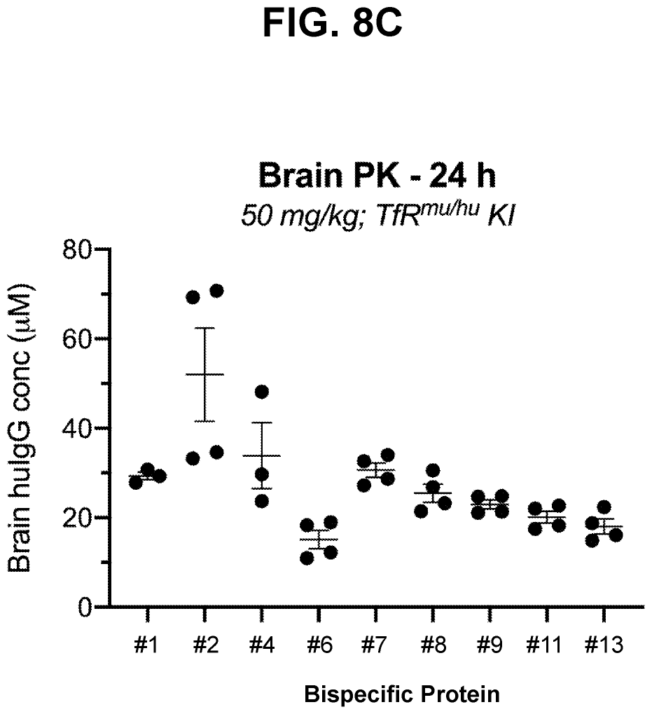

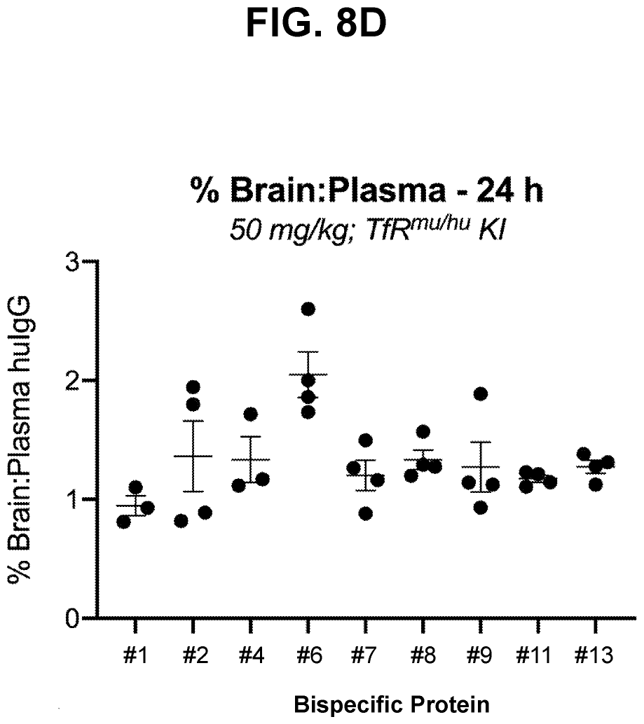

To evaluate the in vivo uptake of anti-HER2 bispecific proteins into the brain, TfR^{mu/hu} KI mice were intravenously administered 50 mg/kg of anti-HER2 bispecific proteins. Approximately 24 hours after dosing, whole blood was collected into EDTA coated tubes via cardiac puncture, then animals were perfused with ice-cold PBS. Clinical blood chemistry and reticulocyte quantification (FIG. 8A) were evaluated using fresh whole blood and a separate aliquot was processed to plasma. Most anti-HER2 bispecific proteins showed reticulocytes values within the normal range compared to control treated mice.

The plasma and fresh brain were snap-frozen on dry ice and stored at −80° C. Brains were homogenized with 10× volume by tissue weight 1% NP40 buffer in PBS with protease and phosphatase inhibitors using a Qiagen Tissue Lyser II; supernatant was stored at −80° C. Plasma (FIG. 8B) and brain lysate (FIG. 8C) concentrations of anti-HER2 bispecific proteins were quantified using a sandwich ELISA. Plasma concentrations exhibited expected TfR-mediated drug disposition; brain concentrations of anti-HER2 bispecific proteins ranged from approximately 15 nM to 52 nM in the brain, demonstrating the ability of these molecules to bind to TfR on the blood-brain barrier and be transported into the brain. The resulting percentage of brain-to-plasma concentrations were between approximately 1-2% (FIG. 8D), approximately ten-fold higher than expected for antibodies lacking TfR or other receptor-mediated transcytosis targeting. This data demonstrates and supports that numerous anti-HER2 bispecific proteins of different architectures as described herein are brain-penetrant.

### Example 6. In Vitro Growth Inhibition of Anti-HER2 Bispecific Proteins

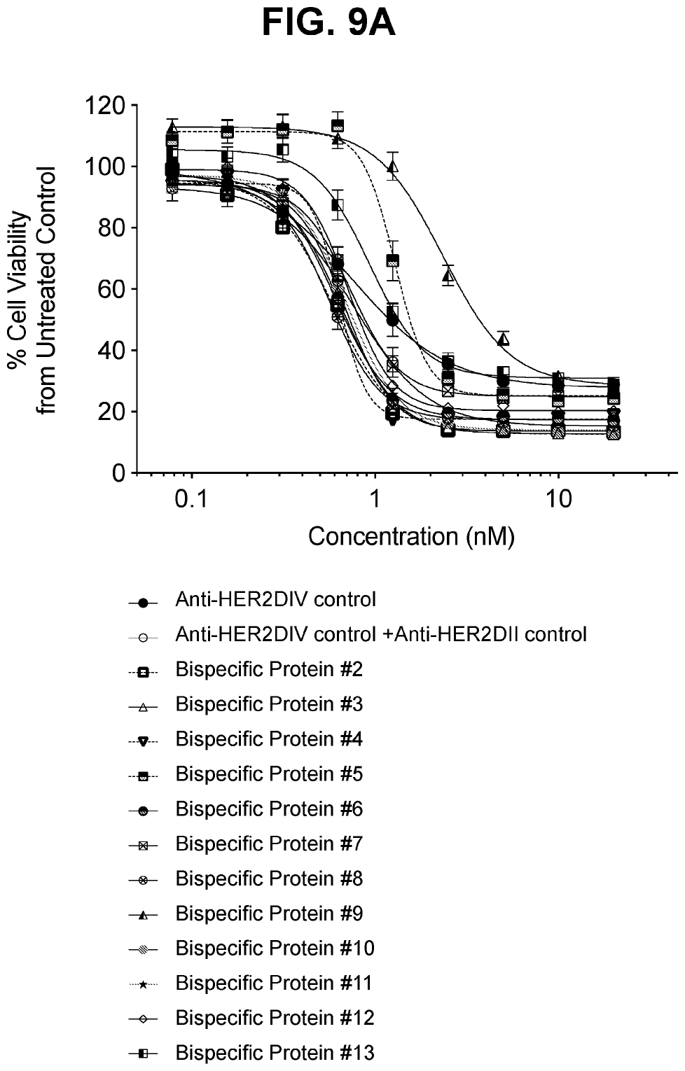

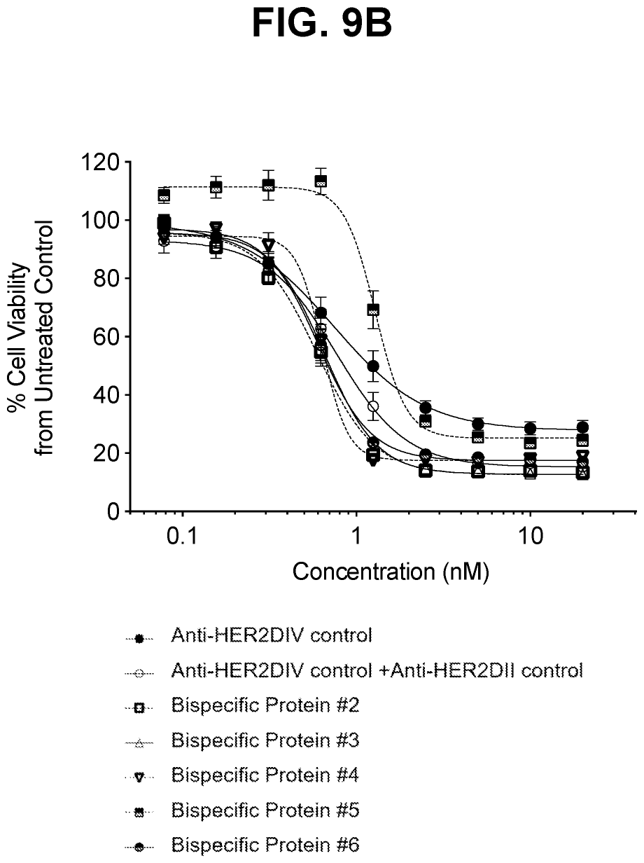

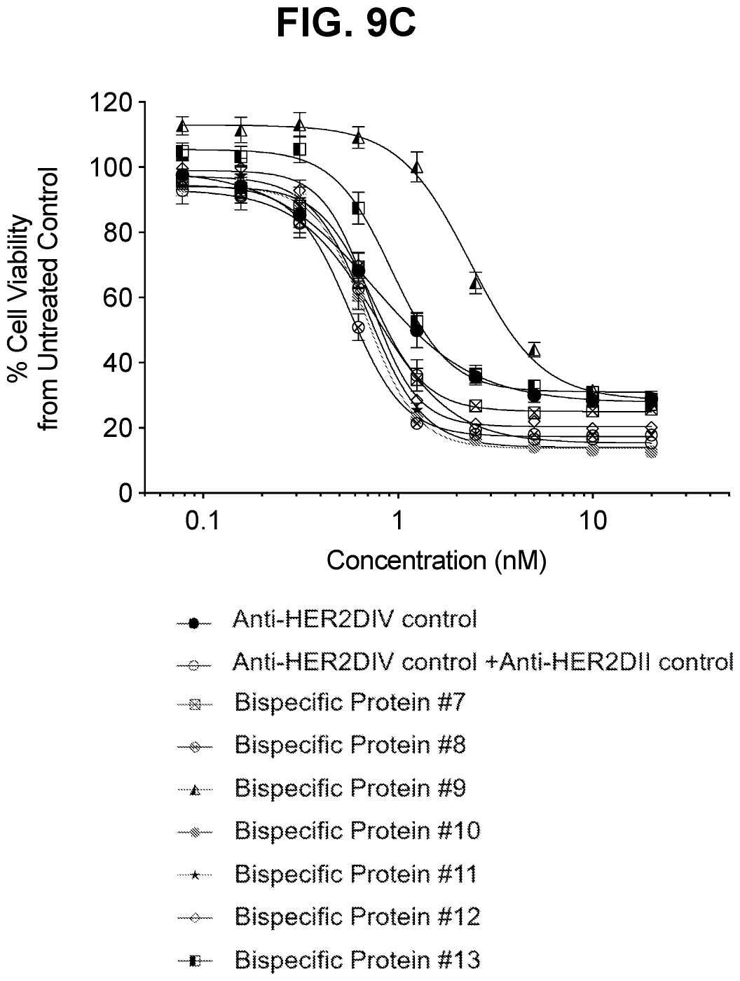

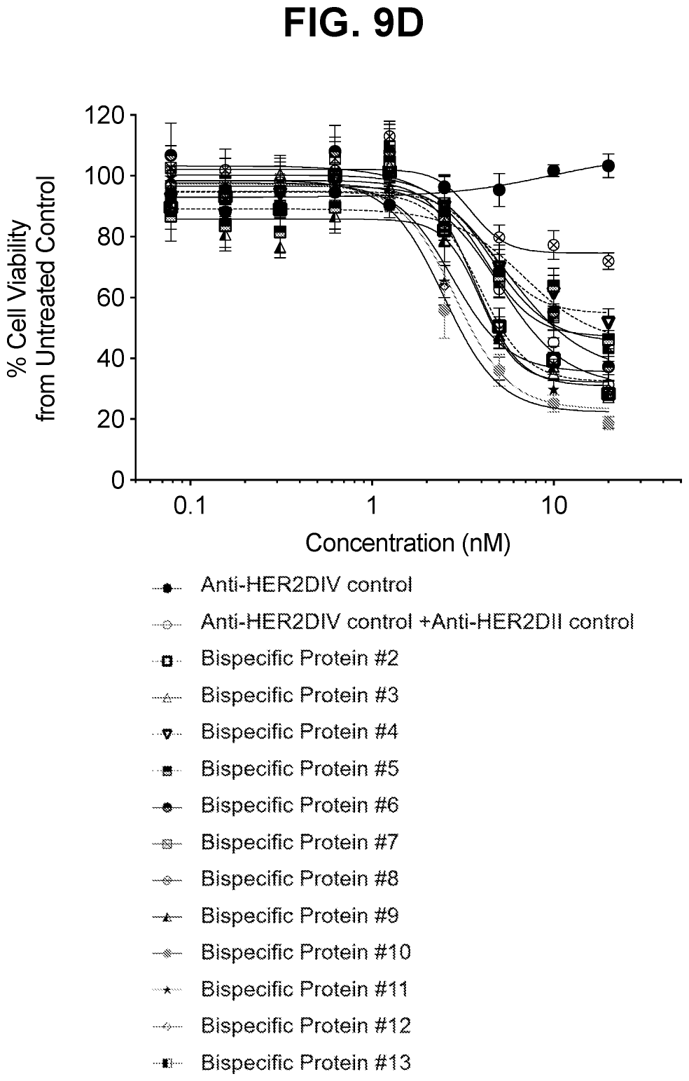

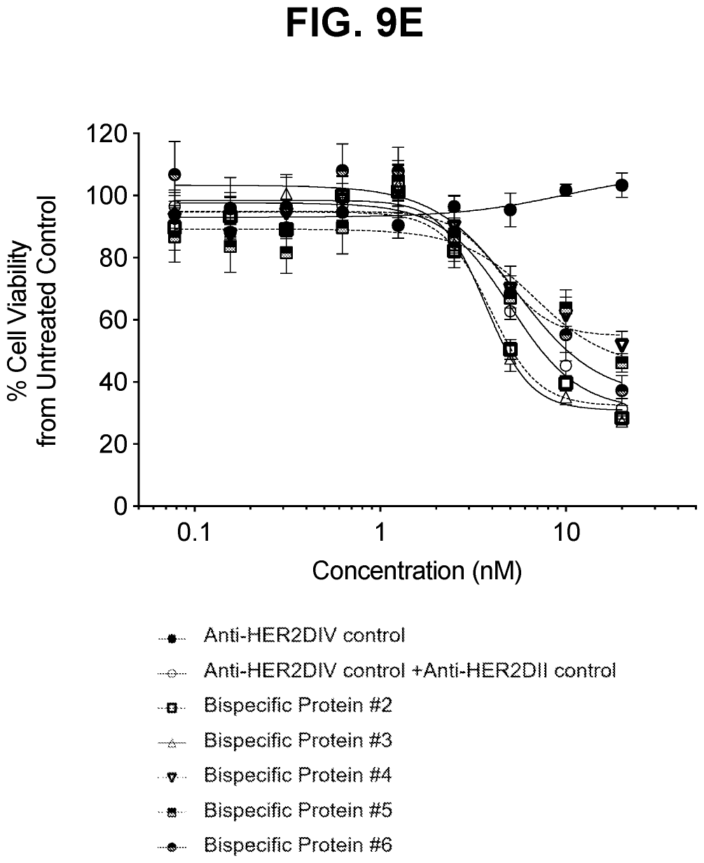

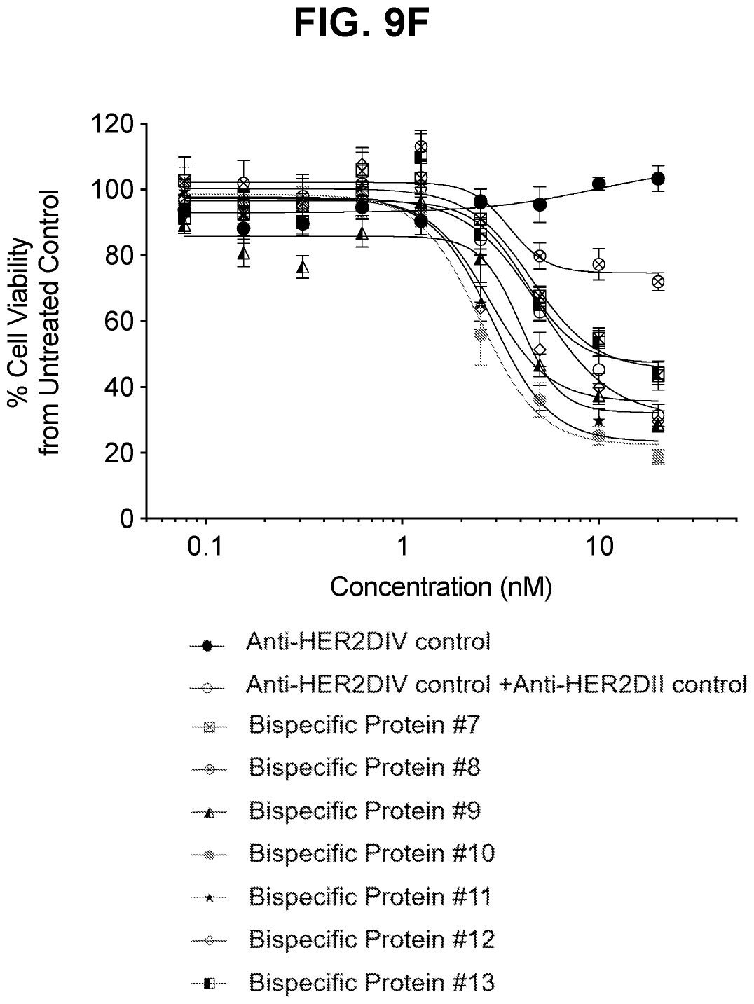

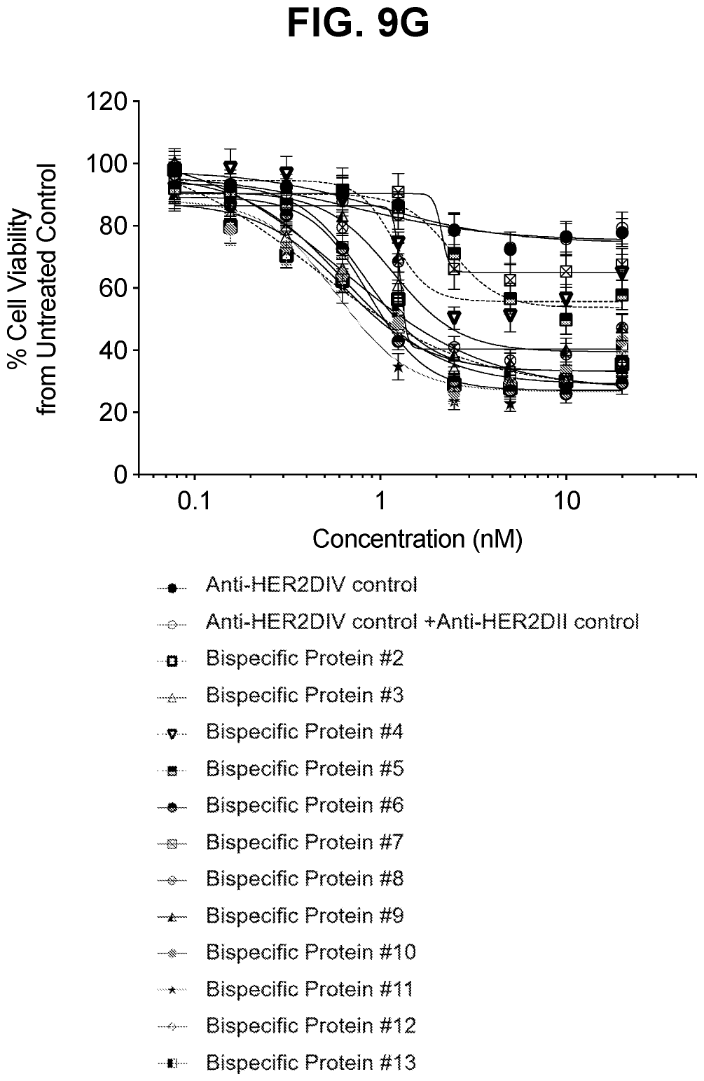

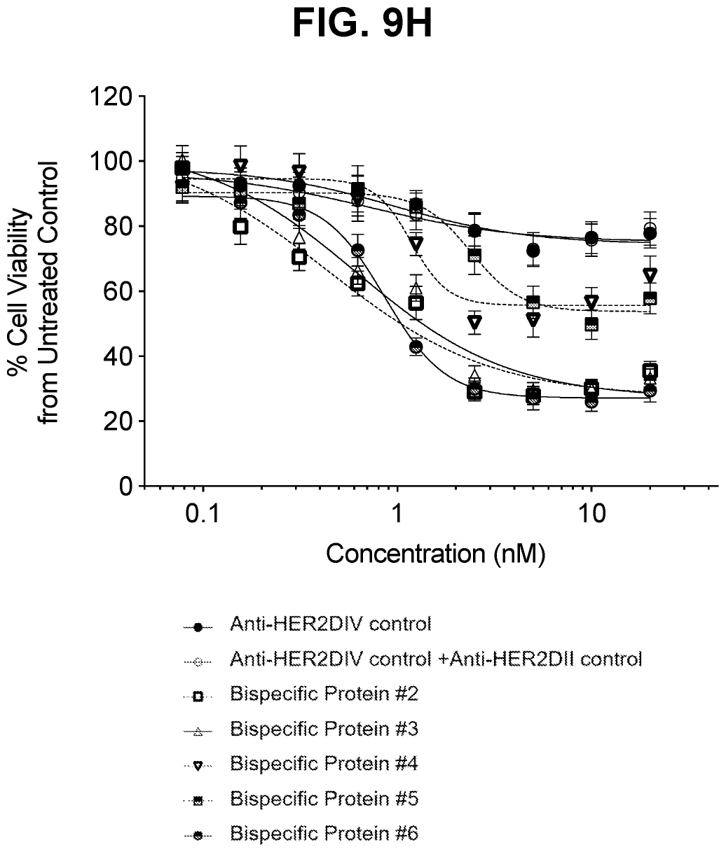

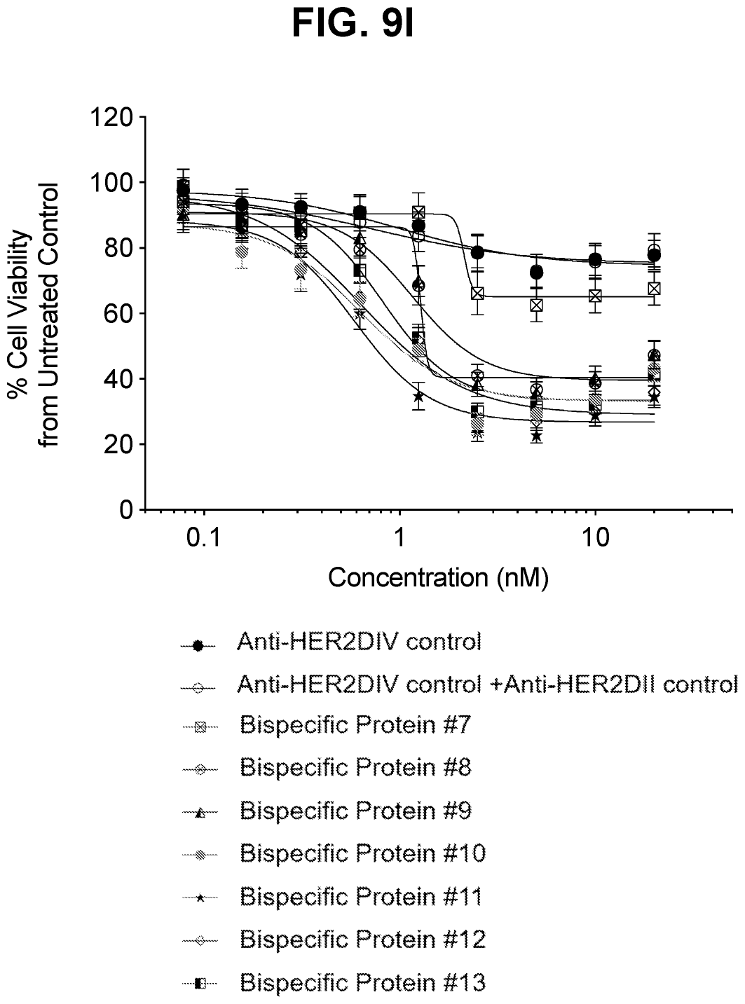

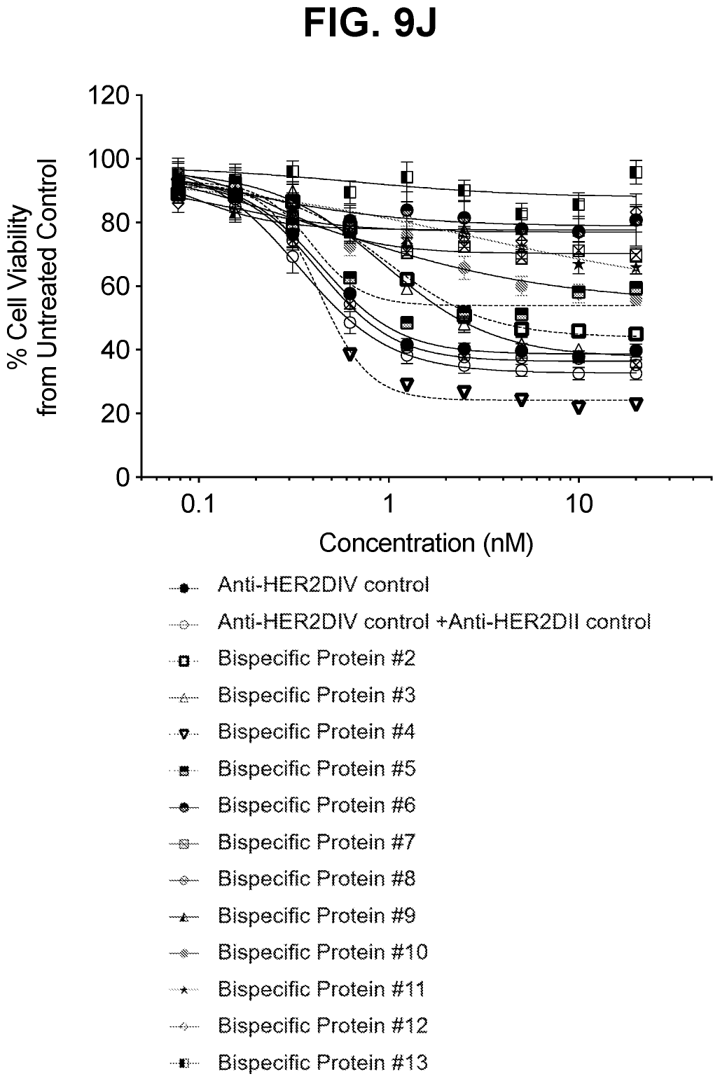

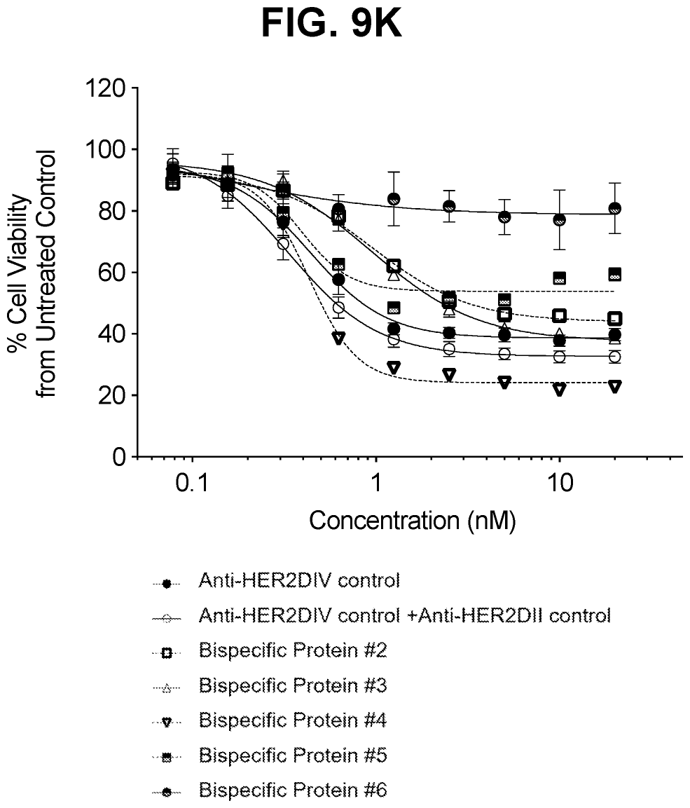

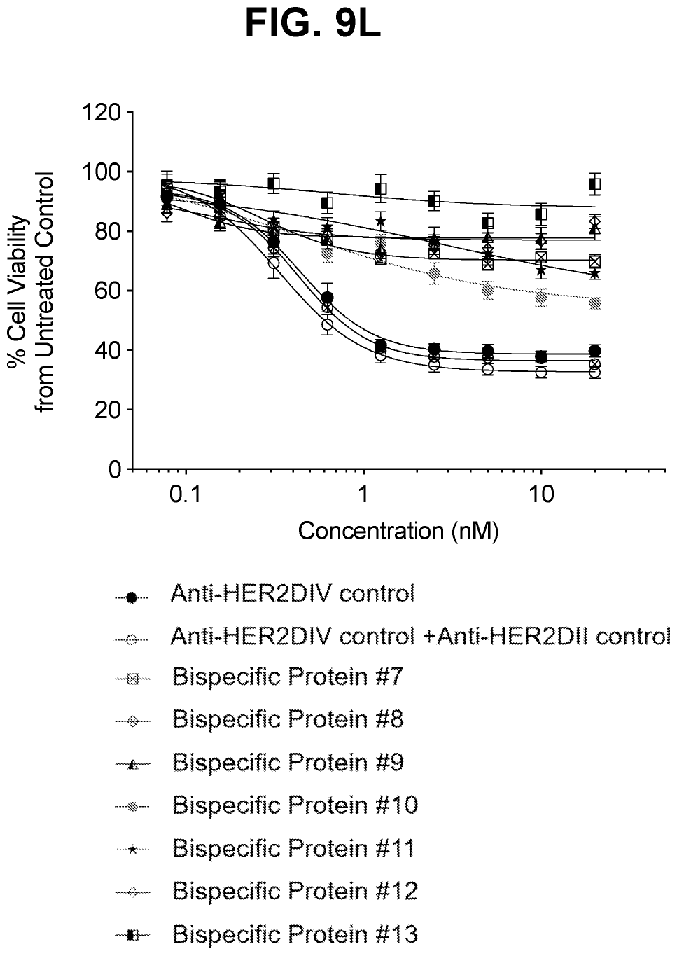

In vitro growth inhibition efficacies of anti-HER2 bispecific proteins and existing anti-HER2 therapies were evaluated with various HER2⁺ tumor cell lines (BT474 (FIGS. 9A-9F), OE19 (FIGS. 9G-9I), and ZR75 (FIGS. 9J-9L)), which are sensitive to anti-HER2 therapies. Tumor cells were plated overnight at 10,000 cells/well in a 96-well plate, treated with 60 μL of 1:3 serial dilution of molecules of interest beginning at 166 nM (25,000 ng/mL). Culture media (RPMI) and drugs were replenished on Day 3. For the neuregulin 1 (NRG1) treated BT474-group (FIGS. 9D-9F), cells were incubated in the presence of 50 ng/ml of NRG1. On Day 6, cell growth was determined using 5 μL of WST-1 reagent (Sigma Aldrich) in 50 μL of growth media. The plate was incubated for 4 hours in the presence of WST-1 reagent, and absorbance was determined at 440 nm. The percent of growth inhibition/proliferation was calculated based on A440 nM and was normalized to the untreated control. Table 3 below further shows the growth inhibition efficacies of the anti-HER2 bispecific proteins and anti-HER2 controls in the three different HER2⁺ cell lines. FIGS. 9A-9L and Table 3 show that overall anti-HER2 bispecific proteins show equivalent or superior growth inhibition than anti-HER2DIV control and anti-HER2DII control with wild-type Fc. In some cell lines with certain anti-HER2 bispecific proteins, the growth inhibition was reduced compared to anti-HER2DIV control or anti-HER2DII control with wild-type Fc, possibly due to competition for binding sites in certain configurations.

TABLE 3

| | BT-474 | | BT474 + NRG1 | | OE19 | | ZR75-30 | |
|---|---|---|---|---|---|---|---|---|
| | Min Cell | | Min Cell | | Min Cell | | Min Cell | |
| | viability | IC50 | viability | IC50 | viability | IC50 | viability | IC50 |
| | (%) | (nM) | (%) | (nM) | (%) | (nM) | (%) | (nM) |
| Anti-HER2DIV control | 34 | 0.76 | 92 | ND | 82 | ND | 52 | 0.42 |
| Anti-HER2DII control | 80 | ND* | 93 | ND | 95 | ND | 81 | ND |
| Anti-HER2DIV control + | 28 | 0.77 | 36 | 5.0 | 83 | ND | 41 | 0.34 |
| Anti-HER2DII control | | | | | | | | |
| Bispecific Protein #1 | 35 | 0.62 | ND | ND | 40 | 0.56 | 97 | ND |
| Bispecific Protein #2 | 22 | 0.59 | 38 | 4.0 | 26 | 0.42 | 61 | 1.2 |
| Bispecific Protein #3 | 23 | 0.68 | 36 | 3.7 | 26 | 0.65 | 52 | 1.2 |
| Bispecific Protein #4 | 28 | 0.64 | 59 | 8.2 | 64 | 1.1 | 40 | 0.43 |
| Bispecific Protein #5 | 19 | 1.3 | 50 | 12 | 66 | 3.1 | 66 | 0.82 |
| Bispecific Protein #6 | 26 | 0.61 | 40 | 5.1 | 42 | 0.86 | 85 | ND |
| Bispecific Protein #7 | 35 | 0.74 | 50 | 4.6 | 76 | ND | 75 | ND |
| Bispecific Protein #8 | 28 | 0.56 | 78 | 4.0 | 53 | 1.2 | 49 | 0.43 |
| Bispecific Protein #9 | 21 | 2.3 | 37 | 3.5 | 57 | 1.1 | 87 | ND |
| Bispecific Protein #10 | 25 | 0.69 | 29 | 2.4 | 49 | 0.64 | ND | ND |
| Bispecific Protein #11 | 22 | 0.69 | 41 | 2.9 | 42 | 0.58 | ND | ND |
| Bispecific Protein #12 | 27 | 0.71 | 51 | 2.7 | 39 | 0.69 | 91 | ND |
| Bispecific Protein #13 | 30 | 0.73 | 50 | 4.5 | 44 | 0.82 | 95 | ND |

*ND—Not Determined

### Example 7. Generation of Additional Bispecific Proteins

### Expression and Purification of Recombinant Bispecific Protein Variants

Expression plasmids consisting of (i) a heavy chain polypeptide comprising a TfR-binding site and a knob (T366W) mutation, (ii) a heavy chain polypeptide comprising hole (T366S/L368A/Y407V) mutations, and (iii) light chains according to the combinations in Table 4 and Table 5 are co-transfected in Expi293 or ExpiCHO cells. Recombinant bispecific protein variants are subsequently purified from conditioned media by loading supernatant over a protein A column (GE Mab Select SuRe). The column is washed with 10 column volumes of PBS, pH 7.4. The proteins are eluted with 50 mM sodium citrate, pH 3.0 containing 150 mM NaCl, and immediately neutralized with 200 mM arginine, 137 mM succinic acid, pH 5.0. The proteins are further purified by size-exclusion chromatography (GE Superdex200) using 200 mM arginine, 137 mM succinic acid, pH 5.0 as running buffer. The purified proteins are confirmed by intact mass LC/MS, and purity of >9500 is confirmed by SDS-PAGE and analytical HPLC-SEC.

TABLE 4

| | Heavy Chain | Heavy Chain | |
|---|---|---|---|
| | 1 - K | 2 - H | Light Chain |
| Bispecific Protein #20 | SEQ ID NO: 1 | SEQ ID NO: 22 | SEQ ID NO: 25 |
| Bispecific Protein #21 | SEQ ID NO: 1 | SEQ ID NO: 159 | SEQ ID NO: 25 |
| Bispecific Protein #22 | SEQ ID NO: 164 | SEQ ID NO: 173 | SEQ ID NO: 25 |
| Bispecific Protein #23 | SEQ ID NO: 164 | SEQ ID NO: 174 | SEQ ID NO: 25 |
| Bispecific Protein #24 | SEQ ID NO: 166 | SEQ ID NO: 173 | SEQ ID NO: 25 |
| Bispecific Protein #25 | SEQ ID NO: 165 | SEQ ID NO: 173 | SEQ ID NO: 25 |
| Bispecific Protein #26 | SEQ ID NO: 165 | SEQ ID NO: 174 | SEQ ID NO: 25 |
| Bispecific Protein #27 | SEQ ID NO: 167 | SEQ ID NO: 173 | SEQ ID NO: 25 |
| Bispecific Protein #28 | SEQ ID NO: 164 | SEQ ID NO: 175 | SEQ ID NO: 25 |
| Bispecific Protein #29 | SEQ ID NO: 164 | SEQ ID NO: 176 | SEQ ID NO: 25 |
| Bispecific Protein #30 | SEQ ID NO: 166 | SEQ ID NO: 175 | SEQ ID NO: 25 |
| Bispecific Protein #31 | SEQ ID NO: 165 | SEQ ID NO: 175 | SEQ ID NO: 25 |
| Bispecific Protein #32 | SEQ ID NO: 165 | SEQ ID NO: 176 | SEQ ID NO: 25 |
| Bispecific Protein #33 | SEQ ID NO: 167 | SEQ ID NO: 175 | SEQ ID NO: 25 |

TABLE 5

| | Heavy Chain | Heavy Chain | |
|---|---|---|---|
| | 1 - K | 2 - H | Light Chain |
| Bispecific Protein #34 | SEQ ID NO: 6 | SEQ ID NO: 156 | SEQ ID NO: 26 |
| Bispecific Protein #35 | SEQ ID NO: 6 | SEQ ID NO: 160 | SEQ ID NO: 26 |
| Bispecific Protein #36 | SEQ ID NO: 6 | SEQ ID NO: 161 | SEQ ID NO: 26 |
| Bispecific Protein #37 | SEQ ID NO: 6 | SEQ ID NO: 162 | SEQ ID NO: 26 |
| Bispecific Protein #38 | SEQ ID NO: 169 | SEQ ID NO: 177 | SEQ ID NO: 26 |
| Bispecific Protein #39 | SEQ ID NO: 169 | SEQ ID NO: 178 | SEQ ID NO: 26 |
| Bispecific Protein #40 | SEQ ID NO: 171 | SEQ ID NO: 177 | SEQ ID NO: 26 |
| Bispecific Protein #41 | SEQ ID NO: 170 | SEQ ID NO: 177 | SEQ ID NO: 26 |
| Bispecific Protein #42 | SEQ ID NO: 170 | SEQ ID NO: 178 | SEQ ID NO: 26 |
| Bispecific Protein #43 | SEQ ID NO: 172 | SEQ ID NO: 177 | SEQ ID NO: 26 |
| Bispecific Protein #44 | SEQ ID NO: 169 | SEQ ID NO: 179 | SEQ ID NO: 26 |
| Bispecific Protein #45 | SEQ ID NO: 169 | SEQ ID NO: 180 | SEQ ID NO: 26 |
| Bispecific Protein #46 | SEQ ID NO: 171 | SEQ ID NO: 179 | SEQ ID NO: 26 |
| Bispecific Protein #47 | SEQ ID NO: 170 | SEQ ID NO: 179 | SEQ ID NO: 26 |
| Bispecific Protein #48 | SEQ ID NO: 170 | SEQ ID NO: 180 | SEQ ID NO: 26 |
| Bispecific Protein #49 | SEQ ID NO: 172 | SEQ ID NO: 179 | SEQ ID NO: 26 |
| Bispecific Protein #50 | SEQ ID NO: 169 | SEQ ID NO: 181 | SEQ ID NO: 26 |
| Bispecific Protein #51 | SEQ ID NO: 169 | SEQ ID NO: 182 | SEQ ID NO: 26 |
| Bispecific Protein #52 | SEQ ID NO: 171 | SEQ ID NO: 181 | SEQ ID NO: 26 |
| Bispecific Protein #53 | SEQ ID NO: 170 | SEQ ID NO: 181 | SEQ ID NO: 26 |
| Bispecific Protein #54 | SEQ ID NO: 170 | SEQ ID NO: 182 | SEQ ID NO: 26 |
| Bispecific Protein #55 | SEQ ID NO: 172 | SEQ ID NO: 181 | SEQ ID NO: 26 |

The heavy chain may be further processed during cell culture production, such that the C-terminal lysine residue is removed. Thus, the bispecific proteins listed in Table 4 and Table 5 above may refer to protein molecules comprising heavy chains that are unprocessed (i.e., comprise the C-terminal lysine residue); protein molecules comprising one or more heavy chains that are processed (i.e., the C-terminal lysine residue is absent); or a mixture of protein molecules having processed and/or unprocessed heavy chains.

### Example 8. In Vitro ADCC/ADCP of Anti-HER2 Bispecific Proteins

A human ADCC Reporter Bioassay, V variant kit (Promega G7018) was used to assess activation of human FcγRIIIa, while a human FcgRIIa ADCP Reporter Bioassay kit (Promega G9995) was used to measure activation of the human FcγRIIIa reporter of the bispecific antibodies according to the combinations in Table 6. The kit contains all of the components described below. Several cell lines with varying expression levels of TER2 and TfR were tested. The cells SKBR3 (ATCC HTB-30), ZR-75-30 (ATCC CRL-1504), BT-474 (ATCC HTB-20), OE-19 (Sigma 96071721), CHO-KI+HumanTfR (ChemPartner CRO agreement) were cultured in RPMI (Liffe Technologies 61870-036) supplemented with 10% FBS (Hyclone Bovine serum SH30080.03) and 1% Penicillin-Streptomycin (Life Technologies 15140-122) to exponential phase, washed twice with PBS and resuspended at 1.0×10⁶ cells/mL in RPMI supplemented with 10% FBS and 1% Penicillin/Streptomycin. White 96-well high binding Nunc plates (ThermoFisher) were coated with 25 μL of media containing 50,000 cells/well.

Antibody titrations were prepared in RPMI with 4% low IgG serum and 25 μl per well was added to the plates to opsonize cells, then covered and incubated for 30 minutes at 37° C., 5% CO₂. During antibody opsonization, 3.5 mL of medium was pre-warmed to 37° C. and the FcγR reporter cells were quickly defrosted in 37° C. water bath, without inverting, then added to pre-warmed medium in a 15 mL conical tube with gentle mixing. After 30-minute opsonization, FcγR reporter cell line was added to each plate at 25 μl per well and incubated for 6 hours (hFcγRIIIa and hFcγRIIa activation for SKBR3, ZR-75-30, BT-474) or 16 hours (hFcγRIIIa and hFcγRIIa for CHO-KI+huTfR) at 37° C., 5% CO₂. After incubation, plates were allowed to acclimate to room temperature and 75 μL per well of Bio-Glo luciferase substrate suspension (Promega) was added and luminescence measured on a Perkin Elmer Envision reader. Results are shown in Table 7.

TABLE 6

| | | Hole chain | | | | | | | |
|---|---|---|---|---|---|---|---|---|---|
| | | hole | hole.E | hole.D | hole.DE | hole.PG | hole.PG.D | hole.PG.E | hole.PG.DE |
| Knob | cis-LALA.CH3C.35.23.4.knob | Fc1 | Fc2 | Fc3 | Fc4 | Fc5 | Fc6 | Fc7 | Fc8 |
| chain | cis-LALA.D.CH3C.35.23.4.knob | Fc17 | Fc18 | Fc19 | Fc20 | Fc21 | Fc22 | Fc23 | Fc24 |
| | cis-LALA.E.CH3C.35.23.4.knob | Fc25 | Fc26 | Fc27 | Fc28 | Fc29 | Fc30 | Fc31 | Fc32 |
| | cis-LALA.DE.CH3C.35.23.4.knob | Fc33 | Fc34 | Fc35 | Fc36 | Fc37 | Fc38 | Fc39 | Fc40 |
| | cis-LALAPG.CH3C.35.23.4.knob | Fc41 | Fc42 | Fc43 | Fc44 | Fc45 | Fc46 | Fc47 | Fc48 |
| | cis-LALAPG.D.CH3C.35.23.4.knob | Fc49 | Fc50 | Fc51 | Fc52 | Fc53 | Fc54 | Fc55 | Fc56 |
| | cis-LALAPG.E.CH3C.35.23.4.knob | Fc57 | Fc58 | Fc59 | Fc60 | Fc61 | Fc62 | Fc63 | Fc64 |
| | cis-LALAPG.DE.CH3C.35.23.4.knob | Fc65 | Fc66 | Fc67 | Fc68 | Fc69 | Fc70 | Fc71 | Fc72 |

TABLE 7

| | TIR- | | | | | | |
|---|---|---|---|---|---|---|---|
| | mediated | | | | | | |
| | ADCC | HER2-mediated ADCC | | | ADCP | | |
| | Cell line used | | | | | | |
| Assay done | CHO-hTIR | BT-474 | OE19 | ZR-75-30 | OE19 | ZR-75-30 | SKBR3 |
| Anti-HER2_D4 (control) | 3.0E+04 | 4.3E+05 | 1.4E+06 | 4.8E+05 | 1.1E+05 | 3.6E+04 | 3.5E+04 |
| Fc1 | 2.8E+04 | 1.5E+04 | 4.9E+05 | 3.5E+04 | 9.2E+05 | 1.6E+05 | 3.1E+05 |
| Fc41 | 2.5E+04 | 3.9E+04 | 2.1E+05 | 2.8E+04 | 2.2E+05 | 8.9E+04 | 5.9E+04 |
| Fc5 | 2.7E+04 | 3.3E+04 | 2.2E+05 | 3.0E+04 | | 4.1E+04 | 3.8E+04 |
| Fc45 | 2.6E+04 | 2.3E+04 | 7.1E+04 | 2.4E+04 | 1.5E+05 | 3.6E+04 | 3.3E+04 |
| Fc42 | 2.7E+04 | 1.5E+05 | 6.5E+05 | 7.7E+04 | 2.6E+05 | 1.0E+05 | 5.7E+04 |
| Fc52 | 2.5E+04 | 4.6E+05 | 1.2E+06 | 2.1E+05 | 2.3E+05 | 8.5E+04 | 5.8E+04 |
| Fc44 | 2.6E+04 | 2.0E+05 | 7.3E+05 | 9.3E+04 | 1.6E+05 | 4.1E+04 | 3.9E+04 |
| Fc50 | 2.9E+04 | 3.2E+05 | 2.6E+06 | 1.9E+05 | 1.6E+06 | 3.4E+05 | 2.5E+05 |
| Fc68 | 2.9E+04 | 3.6E+05 | 1.8E+06 | 2.2E+05 | | | |
| Fc7 | 2.7E+04 | 3.1E+04 | 3.0E+05 | 5.0E+04 | | | |
| Fc24 | 1.4E+05 | 2.8E+05 | 1.6E+06 | 1.6E+05 | | | |
| Fc8 | 2.7E+04 | 8.9E+04 | 8.2E+05 | 1.6E+05 | | | |
| Fc40 | 2.5E+05 | 3.4E+05 | 2.0E+06 | 2.3E+05 | | | |
| Fc23 | 7.2E+04 | 1.3E+05 | 1.1E+06 | 7.1E+04 | | | |
| Fc25 | 4.8E+04 | | | 2.2E+05 | | | |
| Fc2 | 2.3E+04 | | | 2.2E+05 | | 1.6E+05 | 3.0E+05 |
| Fc26 | 4.7E+04 | | | 3.4E+05 | | | |

The aim was to develop Fe variants that did not increase TfR-mediated ADCC compared to the control and/or Fc1 and which also had a comparable level of HER2-mediated ADCC as the control and/or improved level of HER2-mediated ADCC levels compared to Fc1. As shown above in Table 7, Fc1, Fc41, Fc5, Fc45, Fc42, Fc52, Fc44, Fc50, Fc68, Fc7, Fc8, Fc2, Fc34, and Fc4 all had a comparable levels of TfR-mediated ADCC in TfR-overexpressing CHO cells as the control. Fc50 and Fc52 showed the highest level of HER2-mediated ADCC across all the tested HER2-overexpressing cell lines without increasing TfR-mediated ADCC activation.

ADCP levels of Fc1, Fc41, Fc5, Fc45, Fc42, Fc52, Fc44, and Fc50 variants are also shown in Table 7 compared to the control across HER2-over expressing cell lines, i.e., OE19, ZR-75-30, and SKBR3.

### Example 9 In Vitro Growth Inhibition of Anti-HER2 Bispecific Proteins

A Growth Inhibition Assay is used to determine the viability of cells after treatment with different antibodies for different durations. Several cell lines with varying expression levels of HER2 and TfR are tested. The cells SKBR3 (ATCC HTB-30), ZR-75-30 (ATCC CRL-1504), BT-474 (ATCC HTB-20), OE-19 (Sigma 96071721), CHO-KI +HumanTfR (ChemPartner CRO agreement) are cultured in RPMI (Liffe Technologies 61870-036) supplemented with 10% FBS (Hyclone Bovine serum SH30080.03) and 1% Penicillin-Streptomycin (Life Technologies 15140-122) to exponential phase. After washing with PBS, the cells are resuspended at 1.0×10⁵ cells/mL in RPMI supplemented with 10% FBS and 1% Penicillin/Streptomycin. The black Poly-D-Lysine plates (Corning 354640) are coated with 100 ul of cell culture media containing 10,000 cells/well. The plates are incubated for 24 hrs in a 37° C., 5% CO₂ incubator.

Antibody titrations are prepared in RPMI with 10% FBS serum and 1% Penicillin/streptomycin. The antibodies are added to each plate at 65 μl per well, then covered and incubated for 72 hrs (for OE-19 cell line only) and at 37° C., 5% CO₂. For BT-474 and ZR-75-30 cell lines, an additional 65 μl of antibody were added after 72 hrs and then incubated for another 72 hrs at 37° C., 5% CO₂.

On Day 7, cell growth is determined using 5 μL of WST-1 reagent (Sigma Aldrich) in 50 μL of growth media. The plate is incubated for 4 hours in the presence of WST-1 reagent, and absorbance was determined at 440 nm. The percent of growth inhibition/proliferation is calculated based on A440 nM and was normalized to the untreated control.

### X. Exemplary Embodiments

Exemplary embodiments provided in accordance with the presently disclosed subject matter include, but are not limited to, the claims and the following embodiments:
1. A protein comprising:
(a) a first Fc polypeptide that is fused at the N-terminus to an Fd portion of a Fab;
(b) a second Fc polypeptide that is fused at the N-terminus to a single-chain variable fragment (scFv), wherein the first and second Fc polypeptides form an Fc dimer; and
(c) a light chain polypeptide that pairs with the Fd portion recited in (a) to form a Fab,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.
2. The protein of embodiment 1, wherein the first Fc polypeptide and/or the second Fc polypeptide independently comprising the S239D and/or the I332E substitution is capable of enhancing HER2-mediated effector function.
3. The protein of embodiment 1 or 2, wherein:
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.
4. The protein of embodiment 3, wherein:
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.
5. The protein of embodiment 4, wherein:
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at 332.
6. The protein of any one of embodiments 1 to 5, wherein the Fab binds to subdomain II of human HER2 and the scFv binds to subdomain IV of human HER2.
7. The protein of any one of embodiments 1 to 5, wherein the Fab binds to subdomain IV of human HER2 and the scFv binds to subdomain II of human HER2.
8. The protein of any one of embodiments 1 to 7, wherein the second Fc polypeptide is fused to the scFv via a first linker.
9. The protein of embodiment 8, wherein the first linker has a length from 1 to 20 amino acids.
10. The protein of embodiment 8 or 9, wherein the first linker comprises a sequence of any one of GGSGGGSGGGSGGGSGGGSG (SEQ ID NO: 116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).
11. The protein of any one of embodiments 1 to 10, wherein the scFv comprises a V_{L} region and a V_{H} region that are connected via a second linker.
12. The protein of embodiment 11, wherein the second linker has a length from 1 to 20 amino acids.
13. The protein of embodiment 11 or 12, wherein the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).
14. The protein of any one of embodiments 1 to 13, wherein the first Fc polypeptide and/or the second Fc polypeptide specifically binds to a transferrin receptor (TfR).
15. The protein of any one of embodiments 1 to 14, wherein the first Fc polypeptide and the second Fc polypeptide each comprises modifications that promote heterodimerization.
16. The protein of embodiment 15, wherein the first Fc polypeptide comprises a T366W substitution and the second Fc polypeptide comprises T366S, L368A, and Y407V substitutions, according to EU numbering.
17. The protein of embodiment 15, wherein the first Fc polypeptide comprises T366S, L368A, and Y407V substitutions and the second Fc polypeptide comprises a T366W substitution, according to EU numbering.
18. The protein of any one of embodiments 14 to 17, wherein the first Fc polypeptide and/or the second Fc polypeptide independently comprises modifications that reduce TfR-mediated effector function.
19. The protein of embodiment 18, wherein the modifications that reduce effector function are L234A and L235A substitutions, according to EU numbering.
20. The protein of embodiment 19, wherein the first Fc polypeptide specifically binds to TfR and comprises L234A and L235A substitutions.
21. The protein of embodiment 20, wherein the first Fc polypeptide further comprises a P329G or a P329S substitution, according to EU numbering.
22. The protein of embodiment 21, wherein the second Fc polypeptide comprises Leu at positions 234 and 235 and a proline at position 329, according to EU numbering.
23. The protein of embodiment 19, wherein the second Fc polypeptide specifically binds to TfR and comprises L234A and L235A substitutions.
24. The protein of embodiment 23, wherein the second Fc polypeptide further comprises a P329G or a P329S substitution, according to EU numbering.
25. The protein of embodiment 24, wherein the first Fc polypeptide comprises Leu at positions 234 and 235 and a proline at position 329, according to EU numbering.
26. The protein of any one of embodiments 1 to 25, wherein a hinge region or a portion thereof is linked to the N-terminus of the first Fc polypeptide and/or the second Fc polypeptide.
27. The protein of any one of embodiments 1 to 26, wherein the first Fc polypeptide and/or the second Fc polypeptide independently comprises a sequence having at least 90% identity to a sequence selected from the group consisting of SEQ ID NOS:131-149 and 183-196.
28. The protein of embodiment 27, wherein the first Fc polypeptide or the second Fc polypeptide comprises a sequence having at least 90% identity to a sequence selected from SEQ ID NOS:133 and 183-185.
29. The protein of embodiment 27, wherein the first Fc polypeptide or the second Fc polypeptide comprises a sequence having at least 90% identity to a sequence selected from SEQ ID NOS:137 and 186-196.
30. The protein of any one of embodiments 1 to 29, wherein the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to a sequence selected from SEQ ID NOS:135-139 and 186-196.
31. The protein of any one of embodiments 1 to 30, wherein the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137, and
the second Fc polypeptide comprises Ser at position 366, Ala at position 368, and Val at position 407, according to EU numbering, and a sequence having at least 90% identity to the sequence of SEQ ID NO:133.
32. The protein of any one of embodiments 1 to 30, wherein the first Fc polypeptide comprises Ser at position 366, Ala at position 368, and Val at position 407, according to EU numbering, and a sequence having at least 90% identity to the sequence of SEQ ID NO:133, and
the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137.
33. A protein comprising:
(a) (i) a first heavy chain polypeptide comprising a sequence having at least 90% identity to the sequence of SEQ ID NO:1, (ii) a second heavy chain polypeptide comprising a sequence having at least 90% identity to the sequence of SEQ ID NO:159, and (iii) a light chain polypeptide comprising a sequence having at least 90% identity to the sequence of SEQ ID NO:25; or
(b) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(c) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:174, and (iii) a light chain polypeptide comprising a sequence having at least 90% identity to the sequence of SEQ ID NO:25; or
(d) (i) a first heavy chain polypeptide comprising a sequence having at least 90% identity to the sequence of SEQ ID NO: 166, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(e) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(f) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:174, and (iii) a light chain polypeptide comprising a sequence having at least 90% identity to the sequence of SEQ ID NO:25; or
(g) (i) a first heavy chain polypeptide comprising a sequence having at least 90% identity to the sequence of SEQ ID NO: 167, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(h) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(i) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:176, and (iii) a light chain polypeptide comprising a sequence having at least 90% identity to the sequence of SEQ ID NO:25; or
(j) (i) a first heavy chain polypeptide comprising a sequence having at least 90% identity to the sequence of SEQ ID NO: 166, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(k) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(l) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:176, and (iii) a light chain polypeptide comprising a sequence having at least 90% identity to the sequence of SEQ ID NO:25; or
(m) (i) a first heavy chain polypeptide comprising a sequence having at least 90% identity to the sequence of SEQ ID NO: 167, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25.
34. The protein of embodiment 33, wherein:
(a) (i) the first heavy chain polypeptide comprises the sequence of SEQ ID NO:1, (ii) the second heavy chain polypeptide comprises the sequence of SEQ ID NO:159, and (iii) the light chain polypeptide comprises the sequence of SEQ ID NO:25; or
(b) (i) the first heavy chain polypeptide comprises the sequence of SEQ ID NO:164, (ii) the second heavy chain polypeptide comprises the sequence of SEQ ID NO:173, and (iii) the light chain polypeptide comprises the sequence of SEQ ID NO:25; or
(c) (i) the first heavy chain polypeptide comprises the sequence of SEQ ID NO:164, (ii) the second heavy chain polypeptide comprises the sequence of SEQ ID NO:174, and (iii) the light chain polypeptide comprises the sequence of SEQ ID NO:25; or
(d) (i) the first heavy chain polypeptide comprises the sequence of SEQ ID NO:166, (ii) the second heavy chain polypeptide comprises the sequence of SEQ ID NO:173, and (iii) the light chain polypeptide comprises the sequence of SEQ ID NO:25; or
(e) (i) the first heavy chain polypeptide comprises the sequence of SEQ ID NO:165, (ii) the second heavy chain polypeptide comprises the sequence of SEQ ID NO:173, and (iii) the light chain polypeptide comprises the sequence of SEQ ID NO:25; or
(f) (i) the first heavy chain polypeptide comprises the sequence of SEQ ID NO:165, (ii) the second heavy chain polypeptide comprises the sequence of SEQ ID NO:174, and (iii) the light chain polypeptide comprises the sequence of SEQ ID NO:25; or
(g) (i) the first heavy chain polypeptide comprises the sequence of SEQ ID NO:167, (ii) the second heavy chain polypeptide comprises the sequence of SEQ ID NO: 173, and (iii) the light chain polypeptide comprises the sequence of SEQ ID NO:25; or
(h) (i) the first heavy chain polypeptide comprises the sequence of SEQ ID NO:164, (ii) the second heavy chain polypeptide comprises the sequence of SEQ ID NO:175, and (iii) the light chain polypeptide comprises the sequence of SEQ ID NO:25; or
(i) (i) the first heavy chain polypeptide comprises the sequence of SEQ ID NO:164, (ii) the second heavy chain polypeptide comprises the sequence of SEQ ID NO:176, and (iii) the light chain polypeptide comprises the sequence of SEQ ID NO:25; or
(j) (i) the first heavy chain polypeptide comprises the sequence of SEQ ID NO:166, (ii) the second heavy chain polypeptide comprises the sequence of SEQ ID NO:175, and (iii) the light chain polypeptide comprises the sequence of SEQ ID NO:25; or
(k) (i) the first heavy chain polypeptide comprises the sequence of SEQ ID NO:165, (ii) the second heavy chain polypeptide comprises the sequence of SEQ ID NO:175, and (iii) the light chain polypeptide comprises the sequence of SEQ ID NO:25; or
(l) (i) the first heavy chain polypeptide comprises the sequence of SEQ ID NO:165, (ii) the second heavy chain polypeptide comprises the sequence of SEQ ID NO:176, and (iii) the light chain polypeptide comprises the sequence of SEQ ID NO:25; or
(m) (i) the first heavy chain polypeptide comprises the sequence of SEQ ID NO:167, (ii) the second heavy chain polypeptide comprises the sequence of SEQ ID NO:175, and (iii) the light chain polypeptide comprises the sequence of SEQ ID NO:25.
35. A protein comprising:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab,
wherein the Fd portion in (a) and/or (b) is fused at the N-terminus to an scFv,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.
36. The protein of embodiment 35, wherein the first Fc polypeptide and/or the second Fc polypeptide independently comprising the S239D and/or the I332E substitution is capable of enhancing HER2-mediated effector function.
37. The protein of embodiment 35 or 36, wherein:
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.
38. The protein of embodiment 37, wherein:
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.
39. The protein of embodiment 38, wherein:
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at position 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at position 332.
40. The protein of any one of embodiments 35 to 39, wherein the Fab binds to subdomain II of human HER2 and the scFv binds to subdomain IV of human HER2.
41. The protein of any one of embodiments 35 to 39, wherein the Fab binds to subdomain IV of human HER2 and the scFv binds to subdomain II of human HER2.
42. The protein of any one of embodiments 35 to 41, wherein the Fd portion in (a) or (b) is fused at the N-terminus to the scFv.
43. The protein of any one of embodiments 35 to 42, wherein the Fd portion in (a) and/or (b) is fused to the scFv via a first linker.
44. The protein of embodiment 43, wherein the first linker has a length from 1 to 20 amino acids.
45. The protein of embodiment 43 or 44, wherein the first linker comprises a sequence of any one of GGSGGGSGGGSGGGSGGGSG (SEQ ID NO: 116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).
46. The protein of any one of embodiments 35 to 45, wherein the scFv comprises a V_{L} region and a V_{H} region that are connected via a second linker.
47. The protein of embodiment 46, wherein the second linker has a length from 1 to 20 amino acids.
48. The protein of embodiment 46 or 47, wherein the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).
49. The protein of any one of embodiments 35 to 48, wherein the first Fc polypeptide and/or the second Fc polypeptide specifically binds to a transferrin receptor (TfR).
50. The protein of any one of embodiments 35 to 49, wherein the first Fc polypeptide and the second Fc polypeptide each comprises modifications that promote heterodimerization.
51. The protein of embodiment 50, wherein the first Fc polypeptide comprises a T366W substitution and the second Fc polypeptide comprises T366S, L368A, and Y407V substitutions, according to EU numbering.
52. The protein of embodiment 50, wherein the first Fc polypeptide comprises T366S, L368A, and Y407V substitutions and the second Fc polypeptide comprises a T366W substitution, according to EU numbering.
53. The protein of any one of embodiments 49 to 52, wherein the first Fc polypeptide and/or the second Fc polypeptide independently comprises modifications that reduce TfR-mediated effector function.
54. The protein of embodiment 53, wherein the modifications that reduce effector function are L234A and L235A substitutions, according to EU numbering.
55. The protein of embodiment 54, wherein the first Fc polypeptide specifically binds to TfR and comprises L234A and L235A substitutions.
56. The protein of embodiment 55, wherein the first Fc polypeptide further comprises a P329G or a P329S substitution, according to EU numbering.
57. The protein of embodiment 56, wherein the second Fc polypeptide comprises Leu at positions 234 and 235 and proline at position 329, according to EU numbering.
58. The protein of embodiment 54, wherein the second Fc polypeptide specifically binds to TfR and comprises L234A and L235A substitutions.
59. The protein of embodiment 58, wherein the second Fc polypeptide further comprises a P329G or a P329S substitution, according to EU numbering.
60. The protein of embodiment 59, wherein the first Fc polypeptide comprises Leu at positions 234 and 235 and proline at position 329, according to EU numbering.
61. The protein of any one of embodiments 35 to 60, wherein a hinge region or a portion thereof is linked to the N-terminus of the first Fc polypeptide and/or the second Fc polypeptide.
62. The protein of any one of embodiments 35 to 61, wherein the first Fc polypeptide and/or the second Fc polypeptide independently comprises a sequence having at least 90% identity to a sequence selected from the group consisting of SEQ ID NOS:131-149 and 183-196.
63. The protein of embodiment 62, wherein the first Fc polypeptide or the second Fc polypeptide comprises a sequence having at least 90% identity to a sequence selected from SEQ ID NOS:133 and 183-185.
64. The protein of embodiment 62, wherein the first Fc polypeptide or the second Fc polypeptide comprises a sequence having at least 90% identity to a sequence selected from SEQ ID NOS:137 and 186-196.
65. The protein of any one of embodiments 35 to 64, wherein the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to a sequence selected from SEQ ID NOS:135-139 and 186-196.
66. The protein of any one of embodiments 35 to 65, wherein the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137, and
the second Fc polypeptide comprises Ser at position 366, Ala at position 368, and Val at position 407, according to EU numbering, and a sequence having at least 90% identity to the sequence of SEQ ID NO:133.
67. The protein of any one of embodiments 35 to 65, wherein the first Fc polypeptide comprises Ser at position 366, Ala at position 368, and Val at position 407, according to EU numbering, and a sequence having at least 90% identity to the sequence of SEQ ID NO:133, and
the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137.
68. A protein comprising:
(a) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:160, and (iii) two light chain polypeptides each comprising a sequence having at least 90% identity to the sequence of SEQ ID NO:26; or
(b) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:161, and (iii) two light chain polypeptides each comprising a sequence having at least 90% identity to the sequence of SEQ ID NO:26; or
(c) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:162, and (iii) two light chain polypeptides each comprising a sequence having at least 90% identity to the sequence of SEQ ID NO:26; or
(d) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(e) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:178, and (iii) two light chain polypeptides each comprising a sequence having at least 90% identity to the sequence of SEQ ID NO:26; or
(f) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(g) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(h) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:178, and (iii) two light chain polypeptides each comprising a sequence having at least 90% identity to the sequence of SEQ ID NO:26; or
(i) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(j) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(k) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:180, and (iii) two light chain polypeptides each comprising a sequence having at least 90% identity to the sequence of SEQ ID NO:26; or
(l) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(m) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(n) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:180, and (iii) two light chain polypeptides each comprising a sequence having at least 90% identity to the sequence of SEQ ID NO:26; or
(o) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(p) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(q) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:182, and (iii) two light chain polypeptides each comprising a sequence having at least 90% identity to the sequence of SEQ ID NO:26; or
(r) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(s) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(t) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:182, and (iii) two light chain polypeptides each comprising a sequence having at least 90% identity to the sequence of SEQ ID NO:26; or
(u) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26.
69. The protein of embodiment 68, wherein:
(a) (i) the first heavy chain polypeptide comprises the sequence of SEQ ID NO:6, (ii) the second heavy chain polypeptide comprises the sequence of SEQ ID NO:160, and (iii) the two light chain polypeptides each comprises the sequence of SEQ ID NO:26; or
(b) (i) the first heavy chain polypeptide comprises the sequence of SEQ ID NO:6, (ii) the second heavy chain polypeptide comprises the sequence of SEQ ID NO:161, and (iii) the two light chain polypeptides each comprises the sequence of SEQ ID NO:26; or
(c) (i) the first heavy chain polypeptide comprises the sequence of SEQ ID NO:6, (ii) the second heavy chain polypeptide comprises the sequence of SEQ ID NO:162, and (iii) the two light chain polypeptides each comprises the sequence of SEQ ID NO:26; or
(d) (i) the first heavy chain polypeptide comprises the sequence of SEQ ID NO:169, (ii) the second heavy chain polypeptide comprises the sequence of SEQ ID NO:177, and (iii) the two light chain polypeptides each comprises the sequence of SEQ ID NO:26; or
(e) (i) the first heavy chain polypeptide comprises the sequence of SEQ ID NO:169, (ii) the second heavy chain polypeptide comprises the sequence of SEQ ID NO:178, and (iii) the two light chain polypeptides each comprises the sequence of SEQ ID NO:26; or
(f) (i) the first heavy chain polypeptide comprises the sequence of SEQ ID NO:171, (ii) the second heavy chain polypeptide comprises the sequence of SEQ ID NO:177, and (iii) the two light chain polypeptides each comprises the sequence of SEQ ID NO:26; or
(g) (i) the first heavy chain polypeptide comprises the sequence of SEQ ID NO:170, (ii) the second heavy chain polypeptide comprises the sequence of SEQ ID NO:177, and (iii) the two light chain polypeptides each comprises the sequence of SEQ ID NO:26; or
(h) (i) the first heavy chain polypeptide comprises the sequence of SEQ ID NO:170, (ii) the second heavy chain polypeptide comprises the sequence of SEQ ID NO:178, and (iii) the two light chain polypeptides each comprises the sequence of SEQ ID NO:26; or
(i) (i) the first heavy chain polypeptide comprises the sequence of SEQ ID NO:172, (ii) the second heavy chain polypeptide comprises the sequence of SEQ ID NO:177, and (iii) the two light chain polypeptides each comprises the sequence of SEQ ID NO:26; or
(j) (i) the first heavy chain polypeptide comprises the sequence of SEQ ID NO:169, (ii) the second heavy chain polypeptide comprises the sequence of SEQ ID NO:179, and (iii) the two light chain polypeptides each comprises the sequence of SEQ ID NO:26; or
(k) (i) the first heavy chain polypeptide comprises the sequence of SEQ ID NO:169, (ii) the second heavy chain polypeptide comprises the sequence of SEQ ID NO:180, and (iii) the two light chain polypeptides each comprises the sequence of SEQ ID NO:26; or
(l) (i) the first heavy chain polypeptide comprises the sequence of SEQ ID NO:171, (ii) the second heavy chain polypeptide comprises the sequence of SEQ ID NO:179, and (iii) the two light chain polypeptides each comprises the sequence of SEQ ID NO:26; or
(m) (i) the first heavy chain polypeptide comprises the sequence of SEQ ID NO:170, (ii) the second heavy chain polypeptide comprises the sequence of SEQ ID NO:179, and (iii) the two light chain polypeptides each comprises the sequence of SEQ ID NO:26; or
(n) (i) the first heavy chain polypeptide comprises the sequence of SEQ ID NO:170, (ii) the second heavy chain polypeptide comprises the sequence of SEQ ID NO:180, and (iii) the two light chain polypeptides each comprises the sequence of SEQ ID NO:26; or
(o) (i) the first heavy chain polypeptide comprises the sequence of SEQ ID NO:172, (ii) the second heavy chain polypeptide comprises the sequence of SEQ ID NO:179, and (iii) the two light chain polypeptides each comprises the sequence of SEQ ID NO:26; or
(p) (i) the first heavy chain polypeptide comprises the sequence of SEQ ID NO:169, (ii) the second heavy chain polypeptide comprises the sequence of SEQ ID NO:181, and (iii) the two light chain polypeptides each comprises the sequence of SEQ ID NO:26; or
(q) (i) the first heavy chain polypeptide comprises the sequence of SEQ ID NO:169, (ii) the second heavy chain polypeptide comprises the sequence of SEQ ID NO:182, and (iii) the two light chain polypeptides each comprises the sequence of SEQ ID NO:26; or
(r) (i) the first heavy chain polypeptide comprises the sequence of SEQ ID NO:171, (ii) the second heavy chain polypeptide comprises the sequence of SEQ ID NO:181, and (iii) the two light chain polypeptides each comprises the sequence of SEQ ID NO:26; or
(s) (i) the first heavy chain polypeptide comprises the sequence of SEQ ID NO:170, (ii) the second heavy chain polypeptide comprises the sequence of SEQ ID NO:181, and (iii) the two light chain polypeptides each comprises the sequence of SEQ ID NO:26; or
(t) (i) the first heavy chain polypeptide comprises the sequence of SEQ ID NO:170, (ii) the second heavy chain polypeptide comprises the sequence of SEQ ID NO:182, and (iii) the two light chain polypeptides each comprises the sequence of SEQ ID NO:26; or
(u) (i) the first heavy chain polypeptide comprises the sequence of SEQ ID NO:172, (ii) the second heavy chain polypeptide comprises the sequence of SEQ ID NO:181, and (iii) the two light chain polypeptides each comprises the sequence of SEQ ID NO:26.
70. A pharmaceutical composition comprising the protein of any one of embodiments 1 to 69 and a pharmaceutically acceptable carrier.
71. An isolated polynucleotide comprising a nucleotide sequence encoding the protein of any one of embodiments 1 to 69.
72. A vector comprising the polynucleotide of embodiment 71.
73. A host cell comprising the polynucleotide of embodiment 71 or the vector of embodiment 72.
74. A method for treating a cancer or treating brain metastasis of a cancer in a subject, the method comprising administering to the subject a therapeutically effective amount of the protein of any one of embodiments 1 to 69 or the pharmaceutical composition of embodiment 70.
75. The method of embodiment 74, wherein the protein is adminstered in combination with a chemotherapy or radiation therapy.
76. The method of embodiment 74 or 75, wherein the cancer is a metastatic cancer.
77. The method of any one of embodiments 74 to 76, wherein the cancer is a breast cancer.
78. The method of any one of embodiments 74 to 77, wherein the cancer is a HER2 positive cancer.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

INFQRMALSEQUENCE LISTING

| SEQ | | |
|---|---|---|
| ID NO | Sequence | Description |
| 1 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGG | knob LALA |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 2 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with hole |
| | YYCARNLGPSFYFDYWGQGTLVTVSSASTKGPSVFPLAPSSKSTSGGT | LALA |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGK | |
| 3 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with knob LALA |
| | YYCARNLGPSFYFDYWGQGTLVTVSSASTKGPSVFPLAPSSKSTSGGT | |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |
| 4 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA |
| | YYCARNLGPSFYFDYWGQGTLVTVSSASTKGPSVFPLAPSSKSTSGGT | |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGK | |
| 5 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole |
| | YYCARNLGPSFYFDYWGQGTLVTVSSASTKGPSVFPLAPSSKSTSGGT | |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSV | |
| | MHEALHNHYTQKSLSLSPGK | |
| 6 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGK | |
| 7 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with hole |
| | YCSRWGGDGFYAMDYWGQGTLVTVSSASTKGPSVFPLAPSSKSTSGG | LALA |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGK | |
| 8 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with knob LALA |
| | YCSRWGGDGFYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGK | |
| 9 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA |
| | YCSRWGGDGFYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGK | |
| 10 | EVQLVESGGGLVQPGGSLRLSCAASGFNIKDTYIHWVRQAPGKGLEW | Anti-HER2DIV Fab-Fc |
| | VARIYPTNGYTRY ADSVKGRFTISADTSKNTAYLQMNSLRAEDTAVY | with hole |
| | YCSRWGGDGFYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGK | |
| 11 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | CH3C.35.23.4 with |
| | GQGTKVEIKGGSGGGSGGGSGGGSGGGSGEVQLVESGGGLVQPGGSL | knob LALA |
| | RLSCAASGFTFTDYTMDWVRQAPGKGLEWVADVNPNSGGSIYNQRF | (VL-VH scFV) |
| | KGRFTLSVDRSKNTLYLQMNSLRAEDTAVYYCARNLGPSFYFDYWGQ | |
| | GTLVTVSSGGGGSEPKSSDKTHTCPPCPAPEAAGGPSVFLFPPKPKDTL | |
| | MISRTPEVTCVVVDVSHEDPEVKFNWYVDGVEVHNAKTKPREEQYNS | |
| | TYRVVSVLTVLHQDWLNGKEYKCKVSNKALPAPIEKTISKAKGQPREP | |
| | QVYTLPPSRDELTKNQVSLWCLVKGFYPSDIAVEWESYGTEWSNYKT | |
| | TPPVLDSDGSFFLYSKLTVSKEEWQQGFVFSCSVMHEALHNHYTQKSL | |
| | SLSPGK | |
| 12 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSGGSGGGSGGGSGGGSGGGSG | knob LALA |
| | DIQMTQSPSSLSASVGDRVTITCKASQDVSIGVAWYQQKPGKAPKLLI | (VH-VL scFV) |
| | YSASYRYTGVPSRFSGSGSGTDFTLTISSLQPEDFATYYCQQYYIYPYTF | |
| | GQGTKVEIKGGGGSEPKSSDKTHTCPPCPAPEAAGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLWCLVKGFYPSDIAVEWESYGTEWSNYK | |
| | TTPPVLDSDGSFFLYSKLTVSKEEWQQGFVFSCSVMHEALHNHYTQKS | |
| | LSLSPGK | |
| 13 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | CH3C.35.23.4 with hole |
| | GQGTKVEIKGGSGGGSGGGSGGGSGGGSGEVQLVESGGGLVQPGGSL | LALA |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSGGGGSEPKSCDKTHTCPPCPAPEAAGGPSVFLFPPK | |
| | PKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAK | |
| | GQPREPQVYTLPPSRDELTKNQVSLSCAVKGFYPSDIAVEWESYGTEW | |
| | SNYKTTPPVLDSDGSFFLVSKLTVSKEEWQQGFVFSCSVMHEALHNHY | |
| | TQKSLSLSPGK | |
| 14 | DIQMTQSPSSLSASVGDRVTITCKASQDVSIGVAWYQQKPGKAPKLLI | Anti-HER2DII scFv-Fc |
| | YSASYRYTGVPSRFSGSGSGTDFTLTISSLQPEDFATYYCQQYYIYPYTF | with knob LALA |
| | GQGTKVEIKGGSGGGSGGGSGGGSGGGSGEVQLVESGGGLVQPGGSL | |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSGGGGSEPKSCDKTHTCPPCPAPEAAGGPSVFLFPPK | |
| | PKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAK | |
| | GQPREPQVYTLPPSRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSLSLSPGK | |
| 15 | DIQMTQSPSSLSASVGDRVTITCKASQDVSIGVAWYQQKPGKAPKLLI | Anti-HER2DII scFv-Fc |
| | YSASYRYTGVPSRFSGSGSGTDFTLTISSLQPEDFATYYCQQYYIYPYTF | with hole LALA |
| | GQGTKVEIKGGSGGGSGGGSGGGSGGGSGEVQLVESGGGLVQPGGSL | (V_{L}-V_{H} scFV) |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSEPKSSDKTHTCPPCPAPEAAGGPSVFLFPPKPKDTL | |
| | MISRTPEVTCVVVDVSHEDPEVKFNWYVDGVEVHNAKTKPREEQYNS | |
| | TYRVVSVLTVLHQDWLNGKEYKCKVSNKALPAPIEKTISKAKGQPREP | |
| | QVYTLPPSRDELTKNQVSLSCAVKGFYPSDIAVEWESNGQPENNYKTT | |
| | PPVLDSDGSFFLVSKLTVDKSRWQQGNVFSCSVMHEALHNHYTQKSL | |
| | SLSPGK | |
| 16 | DIQMTQSPSSLSASVGDRVTITCKASQDVSIGVAWYQQKPGKAPKLLI | Anti-HER2DII scFv-Fc |
| | YSASYRYTGVPSRFSGSGSGTDFTLTISSLQPEDFATYYCQQYYIYPYTF | with hole |
| | GQGTKVEIKGGSGGGSGGGSGGGSGGGSGEVQLVESGGGLVQPGGSL | (V_{L}-V_{H} scFV) |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSEPKSSDKTHTCPPCPAPELLGGPSVFLFPPKPKDTL | |
| | MISRTPEVTCVVVDVSHEDPEVKFNWYVDGVEVHNAKTKPREEQYNS | |
| | TYRVVSVLTVLHQDWLNGKEYKCKVSNKALPAPIEKTISKAKGQPREP | |
| | QVYTLPPSRDELTKNQVSLSCAVKGFYPSDIAVEWESNGQPENNYKTT | |
| | PPVLDSDGSFFLVSKLTVDKSRWQQGNVFSCSVMHEALHNHYTQKSL | |
| | SLSPGK | |
| 17 | EVQLVESGGGLVQPGGSLRLSCAASGFTFTDYTMDWVRQAPGKGLE | Anti-HER2DII scFv-Fc |
| | WVADVNPNSGGSIYNQRFKGRFTLSVDRSKNTLYLQMNSLRAEDTAV | with hole LALA |
| | YYCARNLGPSFYFDYWGQGTLVTVSSGGSGGGSGGGSGGGSGGGSG | (VH-VL scFV) |
| | DIQMTQSPSSLSASVGDRVTITCKASQDVSIGVAWYQQKPGKAPKLLI | |
| | YSASYRYTGVPSRFSGSGSGTDFTLTISSLQPEDFATYYCQQYYIYPYTF | |
| | GQGTKVEIKGGGGSEPKSSDKTHTCPPCPAPEAAGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLSCAVKGFYPSDIAVEWESNGQPENNYKT | |
| | TPPVLDSDGSFFLVSKLTVDKSRWQQGNVFSCSVMHEALHNHYTQKS | |
| | LSLSPGK | |
| 18 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | CH3C.35.23.4 with |
| | GQGTKVEIKGGSGGGSGGGSGGGSGGGSGEVQLVESGGGLVQPGGSL | knob LALA |
| | RLSCAASGFNIKDTYIHWVRQAPGKGLEWVARIYPTNGYTRYADSVK | (V_{L}-V_{H} scFV) |
| | GRFTISADTSKNTAYLQMNSLRAEDTAVYYCSRWGGDGFYAMDYWG | |
| | QGTLVTVSSGGGGSEPKSSDKTHTCPPCPAPEAAGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLWCLVKGFYPSDIAVEWESYGTEWSNYK | |
| | TTPPVLDSDGSFFLYSKLTVSKEEWQQGFVFSCSVMHEALHNHYTQKS | |
| | LSLSPGK | |
| 19 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | CH3C.35.23.4 with hole |
| | GQGTKVEIKGGSGGGSGGGSGGGSGGGSGEVQLVESGGGLVQPGGSL | LALA |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSGGGGSEPKSCDKTHTCPPCPAPEAAGGPSVFLFPP | |
| | KPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKA | |
| | KGQPREPQVYTLPPSRDELTKNQVSLSCAVKGFYPSDIAVEWESYGTE | |
| | WSNYKTTPPVLDSDGSFFLVSKLTVSKEEWQQGFVFSCSVMHEALHN | |
| | HYTQKSLSLSPGK | |
| 20 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with knob LALA |
| | GQGTKVEIKGGSGGGSGGGSGGGSGGGSGEVQLVESGGGLVQPGGSL | |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSGGGGSEPKSCDKTHTCPPCPAPEAAGGPSVFLFPP | |
| | KPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKA | |
| | KGQPREPQVYTLPPSRDELTKNQVSLWCLVKGFYPSDIAVEWESNGQP | |
| | ENNYKTTPPVLDSDGSFFLYSKLTVDKSRWQQGNVFSCSVMHEALHN | |
| | HYTQKSLSLSPGK | |
| 21 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole LALA |
| | GQGTKVEIKGGSGGGSGGGSGGGSGGGSGEVQLVESGGGLVQPGGSL | (V_{L}-V_{H} scFV) |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSEPKSSDKTHTCPPCPAPEAAGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLSCAVKGFYPSDIAVEWESNGQPENNYKT | |
| | TPPVLDSDGSFFLVSKLTVDKSRWQQGNVFSCSVMHEALHNHYTQKS | |
| | LSLSPGK | |
| 22 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGSGGGSGGGSGGGSGGGSGEVQLVESGGGLVQPGGSL | scFv)_v1 |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSEPKSSDKTHTCPPCPAPELLGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLSCAVKGFYPSDIAVEWESNGQPENNYKT | |
| | TPPVLDSDGSFFLVSKLTVDKSRWQQGNVFSCSVMHEALHNHYTQKS | |
| | LSLSPGK | |
| 23 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GCGTKVEIKGGGGSGGGGSGGGGSGGGGSEVQLVESGGGLVQPGGSL | scFv) (with two Cys |
| | RLSCAASGFNIKDTYIHWVRQAPGKCLEWVARIYPTNGYTRYADSVK | mutations in scFv and |
| | GRFTISADTSKNTAYLQMNSLRAEDTAVYYCSRWGGDGFYAMDYWG | one Cys mutation in |
| | QGTLVTVSSGGGGSEPKSSDKTHTCPPCPAPELLGGPSVFLFPPKPKDT | hinge) |
| | LMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSRDELTKNQVSLSCAVKGFYPSDIAVEWESNGQPENNYKT | |
| | TPPVLDSDGSFFLVSKLTVDKSRWQQGNVFSCSVMHEALHNHYTQKS | |
| | LSLSPGK | |
| 24 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | CH3C.35.23.4 with |
| | GCGTKVEIKGGGGSGGGGSGGGGSGGGGSEVQLVESGGGLVQPGGSL | knob LALA (V_{L}-V_{H} |
| | RLSCAASGFNIKDTYIHWVRQAPGKCLEWVARIYPTNGYTRYADSVK | scFv) (with two Cys |
| | GRFTISADTSKNTAYLQMNSLRAEDTAVYYCSRWGGDGFYAMDYWG | mutations in scFv and |
| | QGTLVTVSSGGGGSEPKSSDKTHTCPPCPAPEAAGGPSVFLFPPKPKDT | one Cys mutation in |
| | LMISRTPEVTCVVVDVSHEDPEVKFNWYVDGVEVHNAKTKPREEQYN | hinge) |
| | STYRVVSVLTVLHQDWLNGKEYKCKVSNKALPAPIEKTISKAKGQPRE | |
| | PQVYTLPPSRDELTKNQVSLWCLVKGFYPSDIAVEWESYGTEWSNYK | |
| | TTPPVLDSDGSFFLYSKLTVSKEEWQQGFVFSCSVMHEALHNHYTQKS | |
| | LSLSPGK | |
| 25 | DIQMTQSPSSLSASVGDRVTITCKASQDVSIGVAWYQQKPGKAPKLLI | LC Anti-HER2DII Fab |
| | YSASYRYTGVPSRFSGSGSGTDFTLTISSLQPEDFATYYCQQYYIYPYTF | |
| | GQGTKVEIKRTVAAPSVFIFPPSDEQLKSGTASVVCLLNNFYPREAKVQ | |
| | WKVDNALQSGNSQESVTEQDSKDSTYSLSSTLTLSKADYEKHKVYAC | |
| | EVTHQGLSSPVTKSFNRGEC | |
| 26 | DIQMTQSPSSLSASVGDRVTITCRASQDVNTAVAWYQQKPGKAPKLLI | LC Anti-HER2DIV Fab |
| | YSASFLYSGVPSRFSGSRSGTDFTLTISSLQPEDFATYYCQQHYTTPPTF | |
| | GQGTKVEIKRTVAAPSVFIFPPSDEQLKSGTASVVCLLNNFYPREAKVQ | |
| | WKVDNALQSGNSQESVTEQDSKDSTYSLSSTLTLSKADYEKHKVYAC | |
| | EVTHQGLSSPVTKSFNRGEC | |
| 27 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV scFv (V_{L}-V_{H} |
| | VPSSSLGTQTYICNVNHKPSNTKVDKKVEPKSCDKTHTCPPCPAPEAA | scFv) |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGSGGGGSDIQMTQSPSSLSASVG | |
| | DRVTITCRASQDVNTAVAWYQQKPGKAPKLLIYSASFLYSGVPSRFSG | |
| | SRSGTDFTLTISSLQPEDFATYYCQQHYTTPPTFGQGTKVEIKGGSGGG | |
| | SGGGSGGGSGGGSGEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSS | |
| 28 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV scFv (VH-VL |
| | VPSSSLGTQTYICNVNHKPSNTKVDKKVEPKSCDKTHTCPPCPAPEAA | scFv) |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGSGGGGSEVQLVESGGGLVQPG | |
| | GSLRLSCAASGFNIKDTYIHWVRQAPGKGLEWVARIYPTNGYTRYADS | |
| | VKGRFTISADTSKNTAYLQMNSLRAEDTAVYYCSRWGGDGFYAMDY | |
| | WGQGTLVTVSSGGSGGGSGGGSGGGSGGGSGDIQMTQSPSSLSASVG | |
| | DRVTITCRASQDVNTAVAWYQQKPGKAPKLLIYSASFLYSGVPSRFSG | |
| | SRSGTDFTLTISSLQPEDFATYYCQQHYTTPPTFGQGTKVEIK | |
| 29 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with hole |
| | YYCARNLGPSFYFDYWGQGTLVTVSSASTKGPSVFPLAPSSKSTSGGT | LALA-anti-HER2DIV |
| | AALGCLVKDYFPEPVTVSWNSGALTSGVHTFPAVLQSSGLYSLSSVVT | scFv |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGSGGGGSDIQMTQSPSSLSASVG | |
| | DRVTITCRASQDVNTAVAWYQQKPGKAPKLLIYSASFLYSGVPSRFSG | |
| | SRSGTDFTLTISSLQPEDFATYYCQQHYTTPPTFGQGTKVEIKGGSGGG | |
| | SGGGSGGGSGGGSGEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSS | |
| 30 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with knob LALA-anti- |
| | YYCARNLGPSFYFDYWGQGTLVTVSSASTKGPSVFPLAPSSKSTSGGT | HER2DIV scFv |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGKGGGGSGGGGSDIQMTQSPSSLSASVG | |
| | DRVTITCRASQDVNTAVAWYQQKPGKAPKLLIYSASFLYSGVPSRFSG | |
| | SRSGTDFTLTISSLQPEDFATYYCQQHYTTPPTFGQGTKVEIKGGSGGG | |
| | SGGGSGGGSGGGSGEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSS | |
| 31 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA-anti- |
| | YYCARNLGPSFYFDYWGQGTLVTVSSASTKGPSVFPLAPSSKSTSGGT | HER2DIV scFv (V_{L}-V_{H} |
| | AALGCLVKDYFPEPVTVSWNSGALTSGVHTFPAVLQSSGLYSLSSVVT | scFv) |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGSGGGGSDIQMTQSPSSLSASVGD | |
| | RVTITCRASQDVNTAVAWYQQKPGKAPKLLIYSASFLYSGVPSRFSGS | |
| | RSGTDFTLTISSLQPEDFATYYCQQHYTTPPTFGQGTKVEIKGGSGGGS | |
| | GGGSGGGSGGGSGEVQLVESGGGLVQPGGSLRLSCAASGFNIKDTYIH | |
| | WVRQAPGKGLEWVARIYPTNGYTRYADSVKGRFTISADTSKNTAYLQ | |
| | MNSLRAEDTAVYYCSRWGGDGFYAMDYWGQGTLVTVSS | |
| 32 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole-anti- |
| | YYCARNLGPSFYFDYWGQGTLVTVSSASTKGPSVFPLAPSSKSTSGGT | HER2DIV scFv (V_{L}-V_{H} |
| | AALGCLVKDYFPEPVTVSWNSGALTSGVHTFPAVLQSSGLYSLSSVVT | scFv) |
| | VPSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSV | |
| | MHEALHNHYTQKSLSLSPGGGGGSGGGGSDIQMTQSPSSLSASVGDR | |
| | VTITCRASQDVNTAVAWYQQKPGKAPKLLIYSASFLYSGVPSRFSGSR | |
| | SGTDFTLTISSLQPEDFATYYCQQHYTTPPTFGQGTKVEIKGGSGGGSG | |
| | GGSGGGSGGGSGEVQLVESGGGLVQPGGSLRLSCAASGFNIKDTYIHW | |
| | VRQAPGKGLEWVARIYPTNGYTRYADSVKGRFTISADTSKNTAYLQM | |
| | NSLRAEDTAVYYCSRWGGDGFYAMDYWGQGTLVTVSS | |
| 33 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA-anti- |
| | TAALGCLVKDYFPEPVTVSWNSGALTSGVHTFPAVLQSSGLYSLSSVV | HER2DII scFv (V_{L}-V_{H} |
| | TVPSSSLGTQTYICNVNHKPSNTKVDKKVEPKSCDKTHTCPPCPAPEA | scFv) |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGKGGGGGSGGGGSDIQMTQSPSSLSAS | |
| | VGDRVTITCKASQDVSIGVAWYQQKPGKAPKLLIYSASYRYTGVPSRF | |
| | SGSGSGTDFTLTISSLQPEDFATYYCQQYYIYPYTFGQGTKVEIKGGSG | |
| | GGSGGGSGGGSGGGSGEVQLVESGGGLVQPGGSLRLSCAASGFTFTD | |
| | YTMDWVRQAPGKGLEWVADVNPNSGGSIYNQRFKGRFTLSVDRSKN | |
| | TLYLQMNSLRAEDTAVYYCARNLGPSFYFDYWGQGTLVTVSS | |
| 34 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA-anti- |
| | TAALGCLVKDYFPEPVTVSWNSGALTSGVHTFPAVLQSSGLYSLSSVV | HER2DII scFv (VH-VL |
| | TVPSSSLGTQTYICNVNHKPSNTKVDKKVEPKSCDKTHTCPPCPAPEA | scFv) |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGKGGGGGSGGGGSEVQLVESGGGLVQ | |
| | PGGSLRLSCAASGFTFTDYTMDWVRQAPGKGLEWVADVNPNSGGSIY | |
| | NQRFKGRFTLSVDRSKNTLYLQMNSLRAEDTAVYYCARNLGPSFYFD | |
| | YWGQGTLVTVSSGGSGGGSGGGSGGGSGGGSGDIQMTQSPSSLSASV | |
| | GDRVTITCKASQDVSIGVAWYQQKPGKAPKLLIYSASYRYTGVPSRFS | |
| | GSGSGTDFTLTISSLQPEDFATYYCQQYYIYPYTFGQGTKVEIK | |
| 35 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with hole |
| | YCSRWGGDGFYAMDYWGQGTLVTVSSASTKGPSVFPLAPSSKSTSGG | LALA-anti-HER2DII |
| | TAALGCLVKDYFPEPVTVSWNSGALTSGVHTFPAVLQSSGLYSLSSVV | SCFv (V_{L}-V_{H} scFV) |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGGSGGGGSDIQMTQSPSSLSASV | |
| | GDRVTITCKASQDVSIGVAWYQQKPGKAPKLLIYSASYRYTGVPSRFS | |
| | GSGSGTDFTLTISSLQPEDFATYYCQQYYIYPYTFGQGTKVEIKGGSGG | |
| | GSGGGSGGGSGGGSGEVQLVESGGGLVQPGGSLRLSCAASGFTFTDYT | |
| | MDWVRQAPGKGLEWVADVNPNSGGSIYNQRFKGRFTLSVDRSKNTL | |
| | YLQMNSLRAEDTAVYYCARNLGPSFYFDYWGQGTLVTVSS | |
| 36 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with knob LALA-anti- |
| | YCSRWGGDGFYAMDYWGQGTLVTVSSASTKGPSVFPLAPSSKSTSGG | HER2DII scFv (V_{L}-V_{H} |
| | TAALGCLVKDYFPEPVTVSWNSGALTSGVHTFPAVLQSSGLYSLSSVV | scFv) |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGKGGGGGSGGGGSDIQMTQSPSSLSAS | |
| | VGDRVTITCKASQDVSIGVAWYQQKPGKAPKLLIYSASYRYTGVPSRF | |
| | SGSGSGTDFTLTISSLQPEDFATYYCQQYYIYPYTFGQGTKVEIKGGSG | |
| | GGSGGGSGGGSGGGSGEVQLVESGGGLVQPGGSLRLSCAASGFTFTD | |
| | YTMDWVRQAPGKGLEWVADVNPNSGGSIYNQRFKGRFTLSVDRSKN | |
| | TLYLQMNSLRAEDTAVYYCARNLGPSFYFDYWGQGTLVTVSS | |
| 37 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA-anti- |
| | YCSRWGGDGFYAMDYWGQGTLVTVSSASTKGPSVFPLAPSSKSTSGG | HER2DII scFv (V_{L}-V_{H} |
| | TAALGCLVKDYFPEPVTVSWNSGALTSGVHTFPAVLQSSGLYSLSSVV | scFv) |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCKASQDVSIGVAWYQQKPGKAPKLLIYSASYRYTGVPSRFSG | |
| | SGSGTDFTLTISSLQPEDFATYYCQQYYIYPYTFGQGTKVEIKGGSGGG | |
| | SGGGSGGGSGGGSGEVQLVESGGGLVQPGGSLRLSCAASGFTFTDYT | |
| | MDWVRQAPGKGLEWVADVNPNSGGSIYNQRFKGRFTLSVDRSKNTL | |
| | YLQMNSLRAEDTAVYYCARNLGPSFYFDYWGQGTLVTVSS | |
| 38 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole-anti-HER2DII |
| | YCSRWGGDGFYAMDYWGQGTLVTVSSASTKGPSVFPLAPSSKSTSGG | SCFv (V_{L}-V_{H} scFV) |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCKASQDVSIGVAWYQQKPGKAPKLLIYSASYRYTGVPSRFSG | |
| | SGSGTDFTLTISSLQPEDFATYYCQQYYIYPYTFGQGTKVEIKGGSGGG | |
| | SGGGSGGGSGGGSGEVQLVESGGGLVQPGGSLRLSCAASGFTFTDYT | |
| | MDWVRQAPGKGLEWVADVNPNSGGSIYNQRFKGRFTLSVDRSKNTL | |
| | YLQMNSLRAEDTAVYYCARNLGPSFYFDYWGQGTLVTVSS | |
| 39 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA-anti- |
| | YCSRWGGDGFYAMDYWGQGTLVTVSSASTKGPSVFPLAPSSKSTSGG | HER2DII scFv (VH-VL |
| | TAALGCLVKDYFPEPVTVSWNSGALTSGVHTFPAVLQSSGLYSLSSVV | scFv) |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSEVQLVESGGGLVQPG | |
| | GSLRLSCAASGFTFTDYTMDWVRQAPGKGLEWVADVNPNSGGSIYNQ | |
| | RFKGRFTLSVDRSKNTLYLQMNSLRAEDTAVYYCARNLGPSFYFDYW | |
| | GQGTLVTVSSGGSGGGSGGGSGGGSGGGSGDIQMTQSPSSLSASVGDR | |
| | VTITCKASQDVSIGVAWYQQKPGKAPKLLIYSASYRYTGVPSRFSGSGS | |
| | GTDFTLTISSLQPEDFATYYCQQYYIYPYTFGQGTKVEIK | |
| 40 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | anti-HER2DII Fab- |
| | GQGTKVEIKGGSGGGSGGGSGGGSGGGSGEVQLVESGGGLVQPGGSL | CH3C.35.23.4 with |
| | RLSCAASGFNIKDTYIHWVRQAPGKGLEWVARIYPTNGYTRYADSVK | knob LALA (V_{L}-V_{H} |
| | GRFTISADTSKNTAYLQMNSLRAEDTAVYYCSRWGGDGFYAMDYWG | scFv) |
| | QGTLVTVSSGGGGSGGGGSEVQLVESGGGLVQPGGSLRLSCAASGFTF | |
| | TDYTMDWVRQAPGKGLEWVADVNPNSGGSIYNQRFKGRFTLSVDRS | |
| | KNTLYLQMNSLRAEDTAVYYCARNLGPSFYFDYWGQGTLVTVSSAST | |
| | KGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTK | |
| | NQVSLWCLVKGFYPSDIAVEWESYGTEWSNYKTTPPVLDSDGSFFLYS | |
| | KLTVSKEEWQQGFVFSCSVMHEALHNHYTQKSLSLSPGK | |
| 41 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | anti-HER2DII Fab- |
| | GQGTKVEIKGGSGGGSGGGSGGGSGGGSGEVQLVESGGGLVQPGGSL | CH3C.35.23.4 with hole |
| | RLSCAASGFNIKDTYIHWVRQAPGKGLEWVARIYPTNGYTRYADSVK | LALA (VL-VH scFv) |
| | GRFTISADTSKNTAYLQMNSLRAEDTAVYYCSRWGGDGFYAMDYWG | |
| | QGTLVTVSSGGGGSGGGGSEVQLVESGGGLVQPGGSLRLSCAASGFTF | |
| | TDYTMDWVRQAPGKGLEWVADVNPNSGGSIYNQRFKGRFTLSVDRS | |
| | KNTLYLQMNSLRAEDTAVYYCARNLGPSFYFDYWGQGTLVTVSSAST | |
| | KGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTK | |
| | NQVSLSCAVKGFYPSDIAVEWESYGTEWSNYKTTPPVLDSDGSFFLVS | |
| | KLTVSKEEWQQGFVFSCSVMHEALHNHYTQKSLSLSPGK | |
| 42 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | anti-HER2DII Fab-Fc |
| | GQGTKVEIKGGSGGGSGGGSGGGSGGGSGEVQLVESGGGLVQPGGSL | with knob LALA |
| | RLSCAASGFNIKDTYIHWVRQAPGKGLEWVARIYPTNGYTRYADSVK | (V_{L}-V_{H} scFV) |
| | GRFTISADTSKNTAYLQMNSLRAEDTAVYYCSRWGGDGFYAMDYWG | |
| | QGTLVTVSSGGGGSGGGGSEVQLVESGGGLVQPGGSLRLSCAASGFTF | |
| | TDYTMDWVRQAPGKGLEWVADVNPNSGGSIYNQRFKGRFTLSVDRS | |
| | KNTLYLQMNSLRAEDTAVYYCARNLGPSFYFDYWGQGTLVTVSSAST | |
| | KGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTK | |
| | NQVSLWCLVKGFYPSDIAVEWESNGQPENNYKTTPPVLDSDGSFFLYS | |
| | KLTVDKSRWQQGNVFSCSVMHEALHNHYTQKSLSLSPGK | |
| 43 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | anti-HER2DII Fab-Fc |
| | GQGTKVEIKGGSGGGSGGGSGGGSGGGSGEVQLVESGGGLVQPGGSL | with hole LALA |
| | RLSCAASGFNIKDTYIHWVRQAPGKGLEWVARIYPTNGYTRYADSVK | (V_{L}-V_{H} scFV) |
| | GRFTISADTSKNTAYLQMNSLRAEDTAVYYCSRWGGDGFYAMDYWG | |
| | QGTLVTVSSGGGGSGGGGSEVQLVESGGGLVQPGGSLRLSCAASGFTF | |
| | TDYTMDWVRQAPGKGLEWVADVNPNSGGSIYNQRFKGRFTLSVDRS | |
| | KNTLYLQMNSLRAEDTAVYYCARNLGPSFYFDYWGQGTLVTVSSAST | |
| | KGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTK | |
| | NQVSLSCAVKGFYPSDIAVEWESNGQPENNYKTTPPVLDSDGSFFLVS | |
| | KLTVDKSRWQQGNVFSCSVMHEALHNHYTQKSLSLSPGK | |
| 44 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | anti-HER2DII Fab-Fc |
| | GQGTKVEIKGGSGGGSGGGSGGGSGGGSGEVQLVESGGGLVQPGGSL | with hole (V_{L}-V_{H} SCFv) |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSGGGGSEVQLVESGGGLVQPGGSLRLSCAASGFTF | |
| | TDYTMDWVRQAPGKGLEWVADVNPNSGGSIYNQRFKGRFTLSVDRS | |
| | KNTLYLQMNSLRAEDTAVYYCARNLGPSFYFDYWGQGTLVTVSSAST | |
| | KGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCVVVDV | |
| | SHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTK | |
| | NQVSLSCAVKGFYPSDIAVEWESNGQPENNYKTTPPVLDSDGSFFLVS | |
| | KLTVDKSRWQQGNVFSCSVMHEALHNHYTQKSLSLSPGK | |
| 45 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | anti-HER2DIV Fab- |
| | GQGTKVEIKGGSGGGSGGGSGGGSGGGSGEVQLVESGGGLVQPGGSL | CH3C.35.23.4 with |
| | RLSCAASGFTFTDYTMDWVRQAPGKGLEWVADVNPNSGGSIYNQRF | knob LALA (V_{L}-V_{H} |
| | KGRFTLSVDRSKNTLYLQMNSLRAEDTAVYYCARNLGPSFYFDYWGQ | scFv) |
| | GTLVTVSSGGGGSGGGGSEVQLVESGGGLVQPGGSLRLSCAASGFNIK | |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLWCLVKGFYPSDIAVEWESYGTEWSNYKTTPPVLDSDGSFFL | |
| | YSKLTVSKEEWQQGFVFSCSVMHEALHNHYTQKSLSLSPGK | |
| 46 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab- |
| | YYCARNLGPSFYFDYWGQGTLVTVSSGGSGGGSGGGSGGGSGGGSG | CH3C.35.23.4 with |
| | DIQMTQSPSSLSASVGDRVTITCKASQDVSIGVAWYQQKPGKAPKLLI | knob LALA (VH-VL |
| | YSASYRYTGVPSRFSGSGSGTDFTLTISSLQPEDFATYYCQQYYIYPYTF | scFv) |
| | GQGTKVEIKGGGGSGGGGSEVQLVESGGGLVQPGGSLRLSCAASGFNI | |
| | KDTYIHWVRQAPGKGLEWVARIYPTNGYTRYADSVKGRFTISADTSK | |
| | NTAYLQMNSLRAEDTAVYYCSRWGGDGFYAMDYWGQGTLVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPEAAGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSRDE | |
| | LTKNQVSLWCLVKGFYPSDIAVEWESYGTEWSNYKTTPPVLDSDGSFF | |
| | LYSKLTVSKEEWQQGFVFSCSVMHEALHNHYTQKSLSLSPGK | |
| 47 | EVQLVESGGGLVQPGGSLRLSCAASGFNIKDTYIHWVRQAPGKGLEW | Anti-HER2DII scFv- |
| | VARIYPTNGYTRYADSVKGRFTISADTSKNTAYLQMNSLRAEDTAVY | anti-HER2DIV Fab- |
| | YCSRWGGDGFYAMDYWGQGTLVTVSSASTKGPSVFPLAPSSKSTSGG | CH3C.35.23.4 with hole |
| | TAALGCLVKDYFPEPVTVSWNSGALTSGVHTFPAVLQSSGLYSLSSVV | LALA |
| | TVPSSSLGTQTYICNVNHKPSNTKVDKKVEPKSCDKTHTCPPCP | |
| | APEAAGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVS | |
| | NKALPAPIEKTISKAKGQPREPQVYTLPPSRDELTKNQVSLSCAVKGFY | |
| | PSDIAVEWESYGTEWSNYKTTPPVLDSDGSFFLVSKLTVSKEEWQQGF | |
| | VFSCSVMHEALHNHYTQKSLSLSPGK | |
| 48 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | anti-HER2DIV Fab-Fc |
| | GQGTKVEIKGGSGGGSGGGSGGGSGGGSGEVQLVESGGGLVQPGGSL | with knob LALA |
| | RLSCAASGFTFTDYTMDWVRQAPGKGLEWVADVNPNSGGSIYNQRF | (V_{L}-V_{H} scFV) |
| | KGRFTLSVDRSKNTLYLQMNSLRAEDTAVYYCARNLGPSFYFDYWGQ | |
| | GTLVTVSSGGGGSGGGGSEVQLVESGGGLVQPGGSLRLSCAASGFNIK | |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLWCLVKGFYPSDIAVEWESNGQPENNYKTTPPVLDSDGSFFLY | |
| | SKLTVDKSRWQQGNVFSCSVMHEALHNHYTQKSLSLSPGK | |
| 49 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | anti-HER2DIV Fab-Fc |
| | GQGTKVEIKGGSGGGSGGGSGGGSGGGSGEVQLVESGGGLVQPGGSL | with hole LALA- |
| | RLSCAASGFTFTDYTMDWVRQAPGKGLEWVADVNPNSGGSIYNQRF | (V_{L}-V_{H} scFV) |
| | KGRFTLSVDRSKNTLYLQMNSLRAEDTAVYYCARNLGPSFYFDYWGQ | |
| | GTLVTVSSGGGGSGGGGSEVQLVESGGGLVQPGGSLRLSCAASGFNIK | |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLSCAVKGFYPSDIAVEWESNGQPENNYKTTPPVLDSDGSFFLV | |
| | SKLTVDKSRWQQGNVFSCSVMHEALHNHYTQKSLSLSPGK | |
| 50 | DIQMTQSPSSLSASVGDRVTITCKASQDVSIGVAWYQQKPGKAPKLLI | Anti-HER2DII scFv- |
| | YSASYRYTGVPSRFSGSGSGTDFTLTISSLQPEDFATYYCQQYYIYPYTF | anti-HER2DIV Fab-Fc |
| | GQGTKVEIKGGSGGGSGGGSGGGSGGGSGEVQLVESGGGLVQPGGSL | with hole (V_{L}-V_{H} scFV) |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSGGGGSEVQLVESGGGLVQPGGSLRLSCAASGFNIK | |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLSCAVKGFYPSDIAVEWESNGQPENNYKTTPPVLDSDGSFFLV | |
| | SKLTVDKSRWQQGNVFSCSVMHEALHNHYTQKSLSLSPGK | |
| 51 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole LALA |
| | DIQMTQSPSSLSASVGDRVTITCKASQDVSIGVAWYQQKPGKAPKLLI | (V_{H}-V_{L} scFV) |
| | YSASYRYTGVPSRFSGSGSGTDFTLTISSLQPEDFATYYCQQYYIYPYTF | |
| | GQGTKVEIKGGGGSGGGGSEVQLVESGGGLVQPGGSLRLSCAASGFNI | |
| | KDTYIHWVRQAPGKGLEWVARIYPTNGYTRYADSVKGRFTISADTSK | |
| | NTAYLQMNSLRAEDTAVYYCSRWGGDGFYAMDYWGQGTLVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPEAAGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSRDE | |
| | LTKNQVSLSCAVKGFYPSDIAVEWESNGQPENNYKTTPPVLDSDGSFF | |
| | LVSKLTVDKSRWQQGNVFSCSVMHEALHNHYTQKSLSLSPGK | |
| 52 | DIQMTQSPSSLSASVGDRVTITCKASQDVSIGVAWYQQKPGKAPKLLI | LC anti-HER2DII Fab- |
| | YSASYRYTGVPSRFSGSGSGTDFTLTISSLQPEDFATYYCQQYYIYPYTF | anti-HER2DIV scFv |
| | GQGTKVEIKRTVAAPSVFIFPPSDEQLKSGTASVVCLLNNFYPREAKVQ | (V_{L}-V_{H} scFV) |
| | WKVDNALQSGNSQESVTEQDSKDSTYSLSSTLTLSKADYEKHKVYAC | |
| | EVTHQGLSSPVTKSFNRGECGGGGGSGGGGSDIQMTQSPSSLSASVGD | |
| | RVTITCRASQDVNTAVAWYQQKPGKAPKLLIYSASFLYSGVPSRFSGS | |
| | RSGTDFTLTISSLQPEDFATYYCQQHYTTPPTFGQGTKVEIKGGSGGGS | |
| | GGGSGGGSGGGSGEVQLVESGGGLVQPGGSLRLSCAASGFNIKDTYIH | |
| | WVRQAPGKGLEWVARIYPTNGYTRYADSVKGRFTISADTSKNTAYLQ | |
| | MNSLRAEDTAVYYCSRWGGDGFYAMDYWGQGTLVTVSS | |
| 53 | DIQMTQSPSSLSASVGDRVTITCRASQDVNTAVAWYQQKPGKAPKLLI | LC anti-HER2DIV Fab- |
| | YSASFLYSGVPSRFSGSRSGTDFTLTISSLQPEDFATYYCQQHYTTPPTF | anti-HER2DII scFv |
| | GQGTKVEIKRTVAAPSVFIFPPSDEQLKSGTASVVCLLNNFYPREAKVQ | (V_{L}-V_{H} scFV) |
| | WKVDNALQSGNSQESVTEQDSKDSTYSLSSTLTLSKADYEKHKVYAC | |
| | EVTHQGLSSPVTKSFNRGECGGGGSGGGGSDIQMTQSPSSLSASVGDR | |
| | VTITCKASQDVSIGVAWYQQKPGKAPKLLIYSASYRYTGVPSRFSGSGS | |
| | GTDFTLTISSLQPEDFATYYCQQYYIYPYTFGQGTKVEIKGGSGGGSGG | |
| | GSGGGSGGGSGEVQLVESGGGLVQPGGSLRLSCAASGFTFTDYTMDW | |
| | VRQAPGKGLEWVADVNPNSGGSIYNQRFKGRFTLSVDRSKNTLYLQM | |
| | NSLRAEDTAVYYCARNLGPSFYFDYWGQGTLVTVSS | |
| 54 | DIQMTQSPSSLSASVGDRVTITCRASQDVNTAVAWYQQKPGKAPKLLI | LC anti-HER2DIV Fab- |
| | YSASFLYSGVPSRFSGSRSGTDFTLTISSLQPEDFATYYCQQHYTTPPTF | anti-HER2DII scFv |
| | GQGTKVEIKRTVAAPSVFIFPPSDEQLKSGTASVVCLLNNFYPREAKVQ | (V_{H}-V_{L} scFV) |
| | WKVDNALQSGNSQESVTEQDSKDSTYSLSSTLTLSKADYEKHKVYAC | |
| | EVTHQGLSSPVTKSFNRGECGGGGSGGGGSEVQLVESGGGLVQPGGS | |
| | LRLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGSGGGSGGGSGGGSGGGSGDIQMTQSPSSLSASVGDRVT | |
| | ITCKASQDVSIGVAWYQQKPGKAPKLLIYSASYRYTGVPSRFSGSGSGT | |
| | DFTLTISSLQPEDFATYYCQQYYIYPYTFGQGTKVEIK | |
| 55 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | LC anti-HER2DII Fab |
| | GQGTKVEIKGGSGGGSGGGSGGGSGGGSGEVQLVESGGGLVQPGGSL | (V_{L}-V_{H} scFV) |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSGGGGSDIQMTQSPSSLSASVGDRVTITCKASQDV | |
| | SIGVAWYQQKPGKAPKLLIYSASYRYTGVPSRFSGSGSGTDFTLTISSL | |
| | QPEDFATYYCQQYYIYPYTFGQGTKVEIKRTVAAPSVFIFPPSDEQLKS | |
| | GTASVVCLLNNFYPREAKVQWKVDNALQSGNSQESVTEQDSKDSTYS | |
| | LSSTLTLSKADYEKHKVYACEVTHQGLSSPVTKSFNRGEC | |
| 56 | DIQMTQSPSSLSASVGDRVTITCKASQDVSIGVAWYQQKPGKAPKLLI | Anti-HER2DII scFv-LC |
| | YSASYRYTGVPSRFSGSGSGTDFTLTISSLQPEDFATYYCQQYYIYPYTF | anti-HER2DIV Fab |
| | GQGTKVEIKGGSGGGSGGGSGGGSGGGSGEVQLVESGGGLVQPGGSL | (V_{L}-V_{H} scFV) |
| | RLSCAASGFTFTDYTMDWVRQAPGKGLEWVADVNPNSGGSIYNQRF | |
| | KGRFTLSVDRSKNTLYLQMNSLRAEDTAVYYCARNLGPSFYFDYWGQ | |
| | GTLVTVSSGGGGSGGGGSDIQMTQSPSSLSASVGDRVTITCRASQDVN | |
| | TAVAWYQQKPGKAPKLLIYSASFLYSGVPSRFSGSRSGTDFTLTISSLQ | |
| | PEDFATYYCQQHYTTPPTFGQGTKVEIKRTVAAPSVFIFPPSDEQLKSG | |
| | TASVVCLLNNFYPREAKVQWKVDNALQSGNSQESVTEQDSKDSTYSL | |
| | SSTLTLSKADYEKHKVYACEVTHQGLSSPVTKSFNRGEC | |
| 57 | EVQLVESGGGLVQPGGSLRLSCAASGFTFTDYTMDWVRQAPGKGLE | Anti-HER2DII scFv-LC |
| | WVADVNPNSGGSIYNQRFKGRFTLSVDRSKNTLYLQMNSLRAEDTAV | anti-HER2DIV Fab |
| | YYCARNLGPSFYFDYWGQGTLVTVSSGGSGGGSGGGSGGGSGGGSG | (V_{H}-V_{L} scFV) |
| | DIQMTQSPSSLSASVGDRVTITCKASQDVSIGVAWYQQKPGKAPKLLI | |
| | YSASYRYTGVPSRFSGSGSGTDFTLTISSLQPEDFATYYCQQYYIYPYTF | |
| | GQGTKVEIKGGGGSGGGGSDIQMTQSPSSLSASVGDRVTITCRASQDV | |
| | NTAVAWYQQKPGKAPKLLIYSASFLYSGVPSRFSGSRSGTDFTLTISSL | |
| | QPEDFATYYCQQHYTTPPTFGQGTKVEIKRTVAAPSVFIFPPSDEQLKS | |
| | GTASVVCLLNNFYPREAKVQWKVDNALQSGNSQESVTEQDSKDSTYS | |
| | LSSTLTLSKADYEKHKVYACEVTHQGLSSPVTKSFNRGEC | |
| 58 | EVQLVESGGGLVQPGGSLRLSCAASGFNIKDTYIHWVRQAPGKGLEW | Anti-HER2DIV V_{H}- |
| | VARIYPTNGYTRYADSVKGRFTISADTSKNTAYLQMNSLRAEDTAVY | anti-HER2DII Fab- |
| | YCSRWGGDGFYAMDYWGQGTLVTVSSASTKGPSVFEVQLVESGGGL | CH3C.35.23.4 with |
| | VQPGGSLRLSCAASGFTFTDYTMDWVRQAPGKGLEWVADVNPNSGG | knob LALA |
| | SIYNQRFKGRFTLSVDRSKNTLYLQMNSLRAEDTAVYYCARNLGPSFY | |
| | FDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLWCLVKGFYPSDIAVEWESYGTEWSN | |
| | YKTTPPVLDSDGSFFLYSKLTVSKEEWQQGFVFSCSVMHEALHNHYT | |
| | QKSLSLSPGK | |
| 59 | EVQLVESGGGLVQPGGSLRLSCAASGFNIKDTYIHWVRQAPGKGLEW | Anti-HER2DIV V_{H}- |
| | VARIYPTNGYTRYADSVKGRFTISADTSKNTAYLQMNSLRAEDTAVY | anti-HER2DII Fab- |
| | YCSRWGGDGFYAMDYWGQGTLVTVSSASTKGPSVFEVQLVESGGGL | CH3C.35.23.4 with hole |
| | VQPGGSLRLSCAASGFTFTDYTMDWVRQAPGKGLEWVADVNPNSGG | LALA |
| | SIYNQRFKGRFTLSVDRSKNTLYLQMNSLRAEDTAVYYCARNLGPSFY | |
| | FDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESYGTEWSN | |
| | YKTTPPVLDSDGSFFLVSKLTVSKEEWQQGFVFSCSVMHEALHNHYT | |
| | QKSLSLSPGK | |
| 60 | EVQLVESGGGLVQPGGSLRLSCAASGFNIKDTYIHWVRQAPGKGLEW | Anti-HER2DIV V_{H}- |
| | VARIYPTNGYTRYADSVKGRFTISADTSKNTAYLQMNSLRAEDTAVY | anti-HER2DII Fab-Fc |
| | YCSRWGGDGFYAMDYWGQGTLVTVSSASTKGPSVFEVQLVESGGGL | with knob LALA |
| | VQPGGSLRLSCAASGFTFTDYTMDWVRQAPGKGLEWVADVNPNSGG | |
| | SIYNQRFKGRFTLSVDRSKNTLYLQMNSLRAEDTAVYYCARNLGPSFY | |
| | FDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLWCLVKGFYPSDIAVEWESNGQPENN | |
| | YKTTPPVLDSDGSFFLYSKLTVDKSRWQQGNVFSCSVMHEALHNHYT | |
| | QKSLSLSPGK | |
| 61 | EVQLVESGGGLVQPGGSLRLSCAASGFNIKDTYIHWVRQAPGKGLEW | Anti-HER2DIV V_{H}- |
| | VARIYPTNGYTRYADSVKGRFTISADTSKNTAYLQMNSLRAEDTAVY | anti-HER2DII Fab-Fc |
| | YCSRWGGDGFYAMDYWGQGTLVTVSSASTKGPSVFEVQLVESGGGL | with hole LALA |
| | VQPGGSLRLSCAASGFTFTDYTMDWVRQAPGKGLEWVADVNPNSGG | |
| | SIYNQRFKGRFTLSVDRSKNTLYLQMNSLRAEDTAVYYCARNLGPSFY | |
| | FDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESNGQPENN | |
| | YKTTPPVLDSDGSFFLVSKLTVDKSRWQQGNVFSCSVMHEALHNHYT | |
| | QKSLSLSPGK | |
| 62 | EVQLVESGGGLVQPGGSLRLSCAASGFNIKDTYIHWVRQAPGKGLEW | Anti-HER2DIV V_{H}- |
| | VARIYPTNGYTRYADSVKGRFTISADTSKNTAYLQMNSLRAEDTAVY | anti-HER2DII Fab-Fc |
| | YCSRWGGDGFYAMDYWGQGTLVTVSSASTKGPSVFEVQLVESGGGL | with hole |
| | VQPGGSLRLSCAASGFTFTDYTMDWVRQAPGKGLEWVADVNPNSGG | |
| | SIYNQRFKGRFTLSVDRSKNTLYLQMNSLRAEDTAVYYCARNLGPSFY | |
| | FDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPELLGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESNGQPENN | |
| | YKTTPPVLDSDGSFFLVSKLTVDKSRWQQGNVFSCSVMHEALHNHYT | |
| | QKSLSLSPGK | |
| 63 | EVQLVESGGGLVQPGGSLRLSCAASGFTFTDYTMDWVRQAPGKGLE | Anti-HER2DII V_{H}-anti- |
| | WVADVNPNSGGSIYNQRFKGRFTLSVDRSKNTLYLQMNSLRAEDTAV | HER2DIV Fab- |
| | YYCARNLGPSFYFDYWGQGTLVTVSSASTKGPSVFEVQLVESGGGLV | CH3C.35.23.4 with |
| | QPGGSLRLSCAASGFNIKDTYIHWVRQAPGKGLEWVARIYPTNGYTRY | knob LALA |
| | ADSVKGRFTISADTSKNTAYLQMNSLRAEDTAVYYCSRWGGDGFYA | |
| | MDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLWCLVKGFYPSDIAVEWESYGTEWSN | |
| | YKTTPPVLDSDGSFFLYSKLTVSKEEWQQGFVFSCSVMHEALHNHYT | |
| | QKSLSLSPGK | |
| 64 | EVQLVESGGGLVQPGGSLRLSCAASGFTFTDYTMDWVRQAPGKGLE | Anti-HER2DII V_{H}-anti- |
| | WVADVNPNSGGSIYNQRFKGRFTLSVDRSKNTLYLQMNSLRAEDTAV | HER2DIV Fab- |
| | YYCARNLGPSFYFDYWGQGTLVTVSSASTKGPSVFEVQLVESGGGLV | CH3C.35.23.4 with hole |
| | QPGGSLRLSCAASGFNIKDTYIHWVRQAPGKGLEWVARIYPTNGYTRY | LALA |
| | ADSVKGRFTISADTSKNTAYLQMNSLRAEDTAVYYCSRWGGDGFYA | |
| | MDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESYGTEWSN | |
| | YKTTPPVLDSDGSFFLVSKLTVSKEEWQQGFVFSCSVMHEALHNHYT | |
| | QKSLSLSPGK | |
| 65 | EVQLVESGGGLVQPGGSLRLSCAASGFTFTDYTMDWVRQAPGKGLE | Anti-HER2DII V_{H}-anti- |
| | WVADVNPNSGGSIYNQRFKGRFTLSVDRSKNTLYLQMNSLRAEDTAV | HER2DIV Fab-Fc with |
| | YYCARNLGPSFYFDYWGQGTLVTVSSASTKGPSVFEVQLVESGGGLV | knob LALA |
| | QPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYA | |
| | MDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLWCLVKGFYPSDIAVEWESNGQPENN | |
| | YKTTPPVLDSDGSFFLYSKLTVDKSRWQQGNVFSCSVMHEALHNHYT | |
| | QKSLSLSPGK | |
| 66 | EVQLVESGGGLVQPGGSLRLSCAASGFTFTDYTMDWVRQAPGKGLE | Anti-HER2DII V_{H}-anti- |
| | WVADVNPNSGGSIYNQRFKGRFTLSVDRSKNTLYLQMNSLRAEDTAV | HER2DIV Fab-Fc with |
| | YYCARNLGPSFYFDYWGQGTLVTVSSASTKGPSVFEVQLVESGGGLV | hole LALA |
| | QPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYA | |
| | MDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESNGQPENN | |
| | YKTTPPVLDSDGSFFLVSKLTVDKSRWQQGNVFSCSVMHEALHNHYT | |
| | QKSLSLSPGK | |
| 67 | EVQLVESGGGLVQPGGSLRLSCAASGFTFTDYTMDWVRQAPGKGLE | Anti-HER2DII V_{H}-anti- |
| | WVADVNPNSGGSIYNQRFKGRFTLSVDRSKNTLYLQMNSLRAEDTAV | HER2DIV Fab-Fc with |
| | YYCARNLGPSFYFDYWGQGTLVTVSSASTKGPSVFEVQLVESGGGLV | hole |
| | QPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYA | |
| | MDYWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYIC | |
| | NVNHKPSNTKVDKKVEPKSCDKTHTCPPCPAPELLGGPSVFLFPPKPK | |
| | DTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQ | |
| | PREPQVYTLPPSRDELTKNQVSLSCAVKGFYPSDIAVEWESNGQPENN | |
| | YKTTPPVLDSDGSFFLVSKLTVDKSRWQQGNVFSCSVMHEALHNHYT | |
| | QKSLSLSPGK | |
| 68 | DIQMTQSPSSLSASVGDRVTITCRASQDVNTAVAWYQQKPGKAPKLLI | Anti-HER2DIV V_{L}- |
| | YSASFLYSGVPSRFSGSRSGTDFTLTISSLQPEDFATYYCQQHYTTPPTF | anti-HER2DII Fab- |
| | GQGTKVEIKASTKGPSVFEVQLVESGGGLVQPGGSLRLSCAASGFTFT | CH3C.35.23.4 with |
| | DYTMDWVRQAPGKGLEWVADVNPNSGGSIYNQRFKGRFTLSVDRSK | knob LALA |
| | NTLYLQMNSLRAEDTAVYYCARNLGPSFYFDYWGQGTLVTVSSASTK | |
| | GPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKL | |
| | TVSKEEWQQGFVFSCSVMHEALHNHYTQKSLSLSPGK | |
| 69 | DIQMTQSPSSLSASVGDRVTITCRASQDVNTAVAWYQQKPGKAPKLLI | Anti-HER2DIV V_{L}- |
| | YSASFLYSGVPSRFSGSRSGTDFTLTISSLQPEDFATYYCQQHYTTPPTF | anti-HER2DII Fab- |
| | GQGTKVEIKASTKGPSVFEVQLVESGGGLVQPGGSLRLSCAASGFTFT | CH3C.35.23.4 with hole |
| | DYTMDWVRQAPGKGLEWVADVNPNSGGSIYNQRFKGRFTLSVDRSK | LALA |
| | NTLYLQMNSLRAEDTAVYYCARNLGPSFYFDYWGQGTLVTVSSASTK | |
| | GPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKL | |
| | TVSKEEWQQGFVFSCSVMHEALHNHYTQKSLSLSPGK | |
| 70 | DIQMTQSPSSLSASVGDRVTITCRASQDVNTAVAWYQQKPGKAPKLLI | Anti-HER2DIV V_{L}- |
| | YSASFLYSGVPSRFSGSRSGTDFTLTISSLQPEDFATYYCQQHYTTPPTF | anti-HER2DII Fab-Fc |
| | GQGTKVEIKASTKGPSVFEVQLVESGGGLVQPGGSLRLSCAASGFTFT | with knob LALA |
| | DYTMDWVRQAPGKGLEWVADVNPNSGGSIYNQRFKGRFTLSVDRSK | |
| | NTLYLQMNSLRAEDTAVYYCARNLGPSFYFDYWGQGTLVTVSSASTK | |
| | GPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKL | |
| | TVDKSRWQQGNVFSCSVMHEALHNHYTQKSLSLSPGK | |
| 71 | DIQMTQSPSSLSASVGDRVTITCRASQDVNTAVAWYQQKPGKAPKLLI | Anti-HER2DIV V_{L}- |
| | YSASFLYSGVPSRFSGSRSGTDFTLTISSLQPEDFATYYCQQHYTTPPTF | anti-HER2DII Fab-Fc |
| | GQGTKVEIKASTKGPSVFEVQLVESGGGLVQPGGSLRLSCAASGFTFT | with hole LALA |
| | DYTMDWVRQAPGKGLEWVADVNPNSGGSIYNQRFKGRFTLSVDRSK | |
| | NTLYLQMNSLRAEDTAVYYCARNLGPSFYFDYWGQGTLVTVSSASTK | |
| | GPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLT | |
| | VDKSRWQQGNVFSCSVMHEALHNHYTQKSLSLSPGK | |
| 72 | DIQMTQSPSSLSASVGDRVTITCRASQDVNTAVAWYQQKPGKAPKLLI | Anti-HER2DIV V_{L}- |
| | YSASFLYSGVPSRFSGSRSGTDFTLTISSLQPEDFATYYCQQHYTTPPTF | anti-HER2DII Fab-Fc |
| | GQGTKVEIKASTKGPSVFEVQLVESGGGLVQPGGSLRLSCAASGFTFT | with hole |
| | DYTMDWVRQAPGKGLEWVADVNPNSGGSIYNQRFKGRFTLSVDRSK | |
| | NTLYLQMNSLRAEDTAVYYCARNLGPSFYFDYWGQGTLVTVSSASTK | |
| | GPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLT | |
| | VDKSRWQQGNVFSCSVMHEALHNHYTQKSLSLSPGK | |
| 73 | DIQMTQSPSSLSASVGDRVTITCKASQDVSIGVAWYQQKPGKAPKLLI | Anti-HER2DII V_{L}-anti- |
| | YSASYRYTGVPSRFSGSGSGTDFTLTISSLQPEDFATYYCQQYYIYPYTF | HER2DIV Fab- |
| | GQGTKVEIKASTKGPSVFEVQLVESGGGLVQPGGSLRLSCAASGFNIK | CH3C.35.23.4 with |
| | DTYIHWVRQAPGKGLEWVARIYPTNGYTRYADSVKGRFTISADTSKN | knob LALA |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLWCLVKGFYPSDIAVEWESYGTEWSNYKTTPPVLDSDGSFFL | |
| | YSKLTVSKEEWQQGFVFSCSVMHEALHNHYTQKSLSLSPGK | |
| 74 | DIQMTQSPSSLSASVGDRVTITCKASQDVSIGVAWYQQKPGKAPKLLI | Anti-HER2DII V_{L}-anti- |
| | YSASYRYTGVPSRFSGSGSGTDFTLTISSLQPEDFATYYCQQYYIYPYTF | HER2DIV Fab- |
| | GQGTKVEIKASTKGPSVFEVQLVESGGGLVQPGGSLRLSCAASGFNIK | CH3C.35.23.4 with hole |
| | DTYIHWVRQAPGKGLEWVARIYPTNGYTRYADSVKGRFTISADTSKN | LALA |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLSCAVKGFYPSDIAVEWESYGTEWSNYKTTPPVLDSDGSFFLV | |
| | SKLTVSKEEWQQGFVFSCSVMHEALHNHYTQKSLSLSPGK | |
| 75 | DIQMTQSPSSLSASVGDRVTITCKASQDVSIGVAWYQQKPGKAPKLLI | Anti-HER2DII V_{L}-anti- |
| | YSASYRYTGVPSRFSGSGSGTDFTLTISSLQPEDFATYYCQQYYIYPYTF | HER2DIV Fab-Fc with |
| | GQGTKVEIKASTKGPSVFEVQLVESGGGLVQPGGSLRLSCAASGFNIK | knob LALA |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLWCLVKGFYPSDIAVEWESNGQPENNYKTTPPVLDSDGSFFLY | |
| | SKLTVDKSRWQQGNVFSCSVMHEALHNHYTQKSLSLSPGK | |
| 76 | DIQMTQSPSSLSASVGDRVTITCKASQDVSIGVAWYQQKPGKAPKLLI | Anti-HER2DII V_{L}-anti- |
| | YSASYRYTGVPSRFSGSGSGTDFTLTISSLQPEDFATYYCQQYYIYPYTF | HER2DIV Fab-Fc with |
| | GQGTKVEIKASTKGPSVFEVQLVESGGGLVQPGGSLRLSCAASGFNIK | hole LALA |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPEAAGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLSCAVKGFYPSDIAVEWESNGQPENNYKTTPPVLDSDGSFFLV | |
| | SKLTVDKSRWQQGNVFSCSVMHEALHNHYTQKSLSLSPGK | |
| 77 | DIQMTQSPSSLSASVGDRVTITCKASQDVSIGVAWYQQKPGKAPKLLI | Anti-HER2DII V_{L}-anti- |
| | YSASYRYTGVPSRFSGSGSGTDFTLTISSLQPEDFATYYCQQYYIYPYTF | HER2DIV Fab-Fc with |
| | GQGTKVEIKASTKGPSVFEVQLVESGGGLVQPGGSLRLSCAASGFNIK | hole |
| | DTYIHWVRQAPGKGLEWVARIYPTNGYTRYADSVKGRFTISADTSKN | |
| | TAYLQMNSLRAEDTAVYYCSRWGGDGFYAMDYWGQGTLVTVSSAS | |
| | TKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVE | |
| | PKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELT | |
| | KNQVSLSCAVKGFYPSDIAVEWESNGQPENNYKTTPPVLDSDGSFFLV | |
| | SKLTVDKSRWQQGNVFSCSVMHEALHNHYTQKSLSLSPGK | |
| 78 | DIQMTQSPSSLSASVGDRVTITCRASQDVNTAVAWYQQKPGKAPKLLI | Anti-HER2DIV V_{L}-LC |
| | YSASFLYSGVPSRFSGSRSGTDFTLTISSLQPEDFATYYCQQHYTTPPTF | anti-HER2DII Fab |
| | GQGTKVEIKRTVAAPSVFIDIQMTQSPSSLSASVGDRVTITCKASQDVSI | |
| | GVAWYQQKPGKAPKLLIYSASYRYTGVPSRFSGSGSGTDFTLTISSLQP | |
| | EDFATYYCQQYYIYPYTFGQGTKVEIKRTVAAPSVFIFPPSDEQLKSGT | |
| | ASVVCLLNNFYPREAKVQWKVDNALQSGNSQESVTEQDSKDSTYSLS | |
| | STLTLSKADYEKHKVYACEVTHQGLSSPVTKSFNRGEC | |
| 79 | DIQMTQSPSSLSASVGDRVTITCKASQDVSIGVAWYQQKPGKAPKLLI | Anti-HER2DII V_{L}-LC |
| | YSASYRYTGVPSRFSGSGSGTDFTLTISSLQPEDFATYYCQQYYIYPYTF | anti-HER2DIV Fab |
| | GQGTKVEIKRTVAAPSVFIDIQMTQSPSSLSASVGDRVTITCRASQDVN | |
| | TAVAWYQQKPGKAPKLLIYSASFLYSGVPSRFSGSRSGTDFTLTISSLQ | |
| | PEDFATYYCQQHYTTPPTFGQGTKVEIKRTVAAPSVFIFPPSDEQLKSG | |
| | TASVVCLLNNFYPREAKVQWKVDNALQSGNSQESVTEQDSKDSTYSL | |
| | SSTLTLSKADYEKHKVYACEVTHQGLSSPVTKSFNRGEC | |
| 80 | EVQLVESGGGLVQPGGSLRLSCAASGFNIKDTYIHWVRQAPGKGLEW | Anti-HER2DIV V_{H}-LC |
| | VARIYPTNGYTRYADSVKGRFTISADTSKNTAYLQMNSLRAEDTAVY | anti-HER2DII Fab |
| | YCSRWGGDGFYAMDYWGQGTLVTVSSRTVAAPSVFIDIQMTQSPSSL | |
| | SASVGDRVTITCKASQDVSIGVAWYQQKPGKAPKLLIYSASYRYTGVP | |
| | SRFSGSGSGTDFTLTISSLQPEDFATYYCQQYYIYPYTFGQGTKVEIKRT | |
| | VAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPV | |
| | TKSFNRGEC | |
| 81 | EVQLVESGGGLVQPGGSLRLSCAASGFTFTDYTMDWVRQAPGKGLE | Anti-HER2DII V_{H}-LC |
| | WVADVNPNSGGSIYNQRFKGRFTLSVDRSKNTLYLQMNSLRAEDTAV | anti-HER2DIV Fab |
| | YYCARNLGPSFYFDYWGQGTLVTVSSRTVAAPSVFIDIQMTQSPSSLSA | |
| | SVGDRVTITCRASQDVNTAVAWYQQKPGKAPKLLIYSASFLYSGVPSR | |
| | FSGSRSGTDFTLTISSLQPEDFATYYCQQHYTTPPTFGQGTKVEIKRTVA | |
| | APSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTK | |
| | SFNRGEC | |
| 82 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV V_{H} |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGGSGGGGSEVQLVESGGGLVQP | |
| | GGSLRLSCAASGFNIKDTYIHWVRQAPGKGLEWVARIYPTNGYTRYA | |
| | DSVKGRFTISADTSKNTAYLQMNSLRAEDTAVYYCSRWGGDGFYAM | |
| | DYWGQGTLVTVSS | |
| 83 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV V_{H} |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGGSGGGGGSGGGGSEVQLVESG | |
| | GGLVQPGGSLRLSCAASGFNIKDTYIHWVRQAPGKGLEWVARIYPTN | |
| | GYTRYADSVKGRFTISADTSKNTAYLQMNSLRAEDTAVYYCSRWGG | |
| | DGFYAMDYWGQGTLVTVSS | |
| 84 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with hole |
| | YYCARNLGPSFYFDYWGQGTLVTVSSASTKGPSVFPLAPSSKSTSGGT | LALA-anti-HER2DIV |
| | AALGCLVKDYFPEPVTVSWNSGALTSGVHTFPAVLQSSGLYSLSSVVT | VH |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGGSGGGGGSGGGGSEVQLVESG | |
| | GGLVQPGGSLRLSCAASGFNIKDTYIHWVRQAPGKGLEWVARIYPTN | |
| | GYTRYADSVKGRFTISADTSKNTAYLQMNSLRAEDTAVYYCSRWGG | |
| | DGFYAMDYWGQGTLVTVSS | |
| 85 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with knob LALA-anti- |
| | YYCARNLGPSFYFDYWGQGTLVTVSSASTKGPSVFPLAPSSKSTSGGT | HER2DIV VH |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGKGGGGGSGGGGGSGGGGSEVQLVESG | |
| | GGLVQPGGSLRLSCAASGFNIKDTYIHWVRQAPGKGLEWVARIYPTN | |
| | GYTRYADSVKGRFTISADTSKNTAYLQMNSLRAEDTAVYYCSRWGG | |
| | DGFYAMDYWGQGTLVTVSS | |
| 86 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA-anti- |
| | YYCARNLGPSFYFDYWGQGTLVTVSSASTKGPSVFPLAPSSKSTSGGT | HER2DIV V_{H} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSEVQLVESGGGLVQPG | |
| | GSLRLSCAASGFNIKDTYIHWVRQAPGKGLEWVARIYPTNGYTRYADS | |
| | VKGRFTISADTSKNTAYLQMNSLRAEDTAVYYCSRWGGDGFYAMDY | |
| | WGQGTLVTVSS | |
| 87 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole-anti- |
| | YYCARNLGPSFYFDYWGQGTLVTVSSASTKGPSVFPLAPSSKSTSGGT | HER2DIV V_{H} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSV | |
| | MHEALHNHYTQKSLSLSPGGGGGGSGGGGSEVQLVESGGGLVQPGGS | |
| | LRLSCAASGFNIKDTYIHWVRQAPGKGLEWVARIYPTNGYTRYADSV | |
| | KGRFTISADTSKNTAYLQMNSLRAEDTAVYYCSRWGGDGFYAMDYW | |
| | GQGTLVTVSS | |
| 88 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA-anti- |
| | YYCARNLGPSFYFDYWGQGTLVTVSSASTKGPSVFPLAPSSKSTSGGT | HER2DIV V_{H} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGGSGGGGSEVQLVESGG | |
| | GLVQPGGSLRLSCAASGFNIKDTYIHWVRQAPGKGLEWVARIYPTNG | |
| | YTRYADSVKGRFTISADTSKNTAYLQMNSLRAEDTAVYYCSRWGGD | |
| | GFYAMDYWGQGTLVTVSS | |
| 89 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA-anti- |
| | TAALGCLVKDYFPEPVTVSWNSGALTSGVHTFPAVLQSSGLYSLSSVV | HER2DII V_{H} |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGKGGGGGSGGGGSEVQLVESGGGLVQ | |
| | PGGSLRLSCAASGFTFTDYTMDWVRQAPGKGLEWVADVNPNSGGSIY | |
| | NQRFKGRFTLSVDRSKNTLYLQMNSLRAEDTAVYYCARNLGPSFYFD | |
| | YWGQGTLVTVSS | |
| 90 | VQLVESGGGLVQPGGSLRLSCAASGFNIKDTYIHWVRQAPGKGLEWV | Anti-HER2DIV Fab- |
| | ARIYPTNGYTRYADSVKGRFTISADTSKNTAYLQMNSLRAEDTAVYY | CH3C.35.23.4 with |
| | CSRWGGDGFYAMDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DII V_{H} |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGGSGGGGGSGGGGSEVQLVESG | |
| | GGLVQPGGSLRLSCAASGFTFTDYTMDWVRQAPGKGLEWVADVNPN | |
| | SGGSIYNQRFKGRFTLSVDRSKNTLYLQMNSLRAEDTAVYYCARNLG | |
| | PSFYFDYWGQGTLVTVSS | |
| 91 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with hole |
| | YCSRWGGDGFYAMDYWGQGTLVTVSSASTKGPSVFPLAPSSKSTSGG | LALA-anti-HER2DII |
| | TAALGCLVKDYFPEPVTVSWNSGALTSGVHTFPAVLQSSGLYSLSSVV | VH |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGGSGGGGGSGGGGSEVQLVESG | |
| | GGLVQPGGSLRLSCAASGFTFTDYTMDWVRQAPGKGLEWVADVNPN | |
| | SGGSIYNQRFKGRFTLSVDRSKNTLYLQMNSLRAEDTAVYYCARNLG | |
| | PSFYFDYWGQGTLVTVSS | |
| 92 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with knob LALA-anti- |
| | YCSRWGGDGFYAMDYWGQGTLVTVSSASTKGPSVFPLAPSSKSTSGG | HER2DII VH |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGKGGGGGSGGGGGSGGGGSEVQLVES | |
| | GGGLVQPGGSLRLSCAASGFTFTDYTMDWVRQAPGKGLEWVADVNP | |
| | NSGGSIYNQRFKGRFTLSVDRSKNTLYLQMNSLRAEDTAVYYCARNL | |
| | GPSFYFDYWGQGTLVTVSS | |
| 93 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA-anti- |
| | YCSRWGGDGFYAMDYWGQGTLVTVSSASTKGPSVFPLAPSSKSTSGG | HER2DII VH |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGKGGGGGSGGGGGSGGGGSEVQLVESG | |
| | GGLVQPGGSLRLSCAASGFTFTDYTMDWVRQAPGKGLEWVADVNPN | |
| | SGGSIYNQRFKGRFTLSVDRSKNTLYLQMNSLRAEDTAVYYCARNLG | |
| | PSFYFDYWGQGTLVTVSS | |
| 94 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole-anti-HER2DII |
| | YCSRWGGDGFYAMDYWGQGTLVTVSSASTKGPSVFPLAPSSKSTSGG | VH |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGKGGGGGSGGGGGSGGGGSEVQLVESG | |
| | GGLVQPGGSLRLSCAASGFTFTDYTMDWVRQAPGKGLEWVADVNPN | |
| | SGGSIYNQRFKGRFTLSVDRSKNTLYLQMNSLRAEDTAVYYCARNLG | |
| | PSFYFDYWGQGTLVTVSS | |
| 95 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV V_{L} |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGGSGGGGSDIQMTQSPSSLSASV | |
| | GDRVTITCRASQDVNTAVAWYQQKPGKAPKLLIYSASFLYSGVPSRFS | |
| | GSRSGTDFTLTISSLQPEDFATYYCQQHYTTPPTFGQGTKVEIK | |
| 96 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA-anti- |
| | AALGCLVKDYFPEPVTVSWNSGALTSGVHTFPAVLQSSGLYSLSSVVT | HER2DIV V_{L} |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGKGGGGGSGGGGGSGGGGSDIQMTQSPS | |
| | SLSASVGDRVTITCRASQDVNTAVAWYQQKPGKAPKLLIYSASFLYSG | |
| | VPSRFSGSRSGTDFTLTISSLQPEDFATYYCQQHYTTPPTFGQGTKVEIK | |
| 97 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with hole |
| | YYCARNLGPSFYFDYWGQGTLVTVSSASTKGPSVFPLAPSSKSTSGGT | LALA-anti-HER2DIV |
| | AALGCLVKDYFPEPVTVSWNSGALTSGVHTFPAVLQSSGLYSLSSVVT | VL |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGGSGGGGGSGGGGSDIQMTQSPS | |
| | SLSASVGDRVTITCRASQDVNTAVAWYQQKPGKAPKLLIYSASFLYSG | |
| | VPSRFSGSRSGTDFTLTISSLQPEDFATYYCQQHYTTPPTFGQGTKVEIK | |
| 98 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with knob LALA-anti- |
| | YYCARNLGPSFYFDYWGQGTLVTVSSASTKGPSVFPLAPSSKSTSGGT | HER2DIV VL |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGKGGGGGSGGGGGSGGGGSDIQMTQSP | |
| | SSLSASVGDRVTITCRASQDVNTAVAWYQQKPGKAPKLLIYSASFLYS | |
| | GVPSRFSGSRSGTDFTLTISSLQPEDFATYYCQQHYTTPPTFGQGTKVEI | |
| | K | |
| 99 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA-anti- |
| | YYCARNLGPSFYFDYWGQGTLVTVSSASTKGPSVFPLAPSSKSTSGGT | HER2DIV V_{L} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCRASQDVNTAVAWYQQKPGKAPKLLIYSASFLYSGVPSRFSG | |
| | SRSGTDFTLTISSLQPEDFATYYCQQHYTTPPTFGQGTKVEIK | |
| 100 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole-anti- |
| | YYCARNLGPSFYFDYWGQGTLVTVSSASTKGPSVFPLAPSSKSTSGGT | HER2DIV V_{L} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSV | |
| | MHEALHNHYTQKSLSLSPGGGGGGSGGGGSDIQMTQSPSSLSASVGD | |
| | RVTITCRASQDVNTAVAWYQQKPGKAPKLLIYSASFLYSGVPSRFSGS | |
| | RSGTDFTLTISSLQPEDFATYYCQQHYTTPPTFGQGTKVEIK | |
| 101 | EVQLVESGGGLVQPGGSLRLSCAASGFTFTDYTMDWVRQAPGKGLE | Anti-HER2DII Fab-Fc |
| | WVADVNPNSGGSIYNQRFKGRFTLSVDRSKNTLYLQMNSLRAEDTAV | with hole LALA-anti- |
| | YYCARNLGPSFYFDYWGQGTLVTVSSASTKGPSVFPLAPSSKSTSGGT | HER2DIV V_{L} |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGGSGGGGSDIQMTQSPS | |
| | SLSASVGDRVTITCRASQDVNTAVAWYQQKPGKAPKLLIYSASFLYSG | |
| | VPSRFSGSRSGTDFTLTISSLQPEDFATYYCQQHYTTPPTFGQGTKVEIK | |
| 102 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA-anti- |
| | TAALGCLVKDYFPEPVTVSWNSGALTSGVHTFPAVLQSSGLYSLSSVV | HER2DII VL |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGKGGGGGSGGGGSDIQMTQSPSSLSAS | |
| | VGDRVTITCKASQDVSIGVAWYQQKPGKAPKLLIYSASYRYTGVPSRF | |
| | SGSGSGTDFTLTISSLQPEDFATYYCQQYYIYPYTFGQGTKVEIK | |
| 103 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with hole |
| | YCSRWGGDGFYAMDYWGQGTLVTVSSASTKGPSVFPLAPSSKSTSGG | LALA-anti-HER2DII |
| | TAALGCLVKDYFPEPVTVSWNSGALTSGVHTFPAVLQSSGLYSLSSVV | VL |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESYGTEWSNYKTTPPVLDSDGSFFLVSKLTVSKEEWQQGFVFSCS | |
| | VMHEALHNHYTQKSLSLSPGKGGGGGSGGGGSDIQMTQSPSSLSASV | |
| | GDRVTITCKASQDVSIGVAWYQQKPGKAPKLLIYSASYRYTGVPSRFS | |
| | GSGSGTDFTLTISSLQPEDFATYYCQQYYIYPYTFGQGTKVEIK | |
| 104 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with knob LALA-anti- |
| | YCSRWGGDGFYAMDYWGQGTLVTVSSASTKGPSVFPLAPSSKSTSGG | HER2DII VL |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGKGGGGGSGGGGSDIQMTQSPSSLSAS | |
| | VGDRVTITCKASQDVSIGVAWYQQKPGKAPKLLIYSASYRYTGVPSRF | |
| | SGSGSGTDFTLTISSLQPEDFATYYCQQYYIYPYTFGQGTKVEIK | |
| 105 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA-anti- |
| | YCSRWGGDGFYAMDYWGQGTLVTVSSASTKGPSVFPLAPSSKSTSGG | HER2DII V_{L} |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCKASQDVSIGVAWYQQKPGKAPKLLIYSASYRYTGVPSRFSG | |
| | SGSGTDFTLTISSLQPEDFATYYCQQYYIYPYTFGQGTKVEIK | |
| 106 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole-anti-HER2DII |
| | YCSRWGGDGFYAMDYWGQGTLVTVSSASTKGPSVFPLAPSSKSTSGG | V_{L} |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCKASQDVSIGVAWYQQKPGKAPKLLIYSASYRYTGVPSRFSG | |
| | SGSGTDFTLTISSLQPEDFATYYCQQYYIYPYTFGQGTKVEIK | |
| 107 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole LALA-anti- |
| | YCSRWGGDGFYAMDYWGQGTLVTVSSASTKGPSVFPLAPSSKSTSGG | HER2DII V_{L} |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGGSGGGGSDIQMTQSPS | |
| | SLSASVGDRVTITCKASQDVSIGVAWYQQKPGKAPKLLIYSASYRYTG | |
| | VPSRFSGSGSGTDFTLTISSLQPEDFATYYCQQYYIYPYTFGQGTKVEIK | |
| 108 | EVQLVESGGGLVQPGGSLRLSCAASGFTFTDYTMDWVRQAPGKGLE | Anti-HER2DII V_{H} |
| | WVADVNPNSGGSIYNQRFKGRFTLSVDRSKNTLYLQMNSLRAEDTAV | region |
| | YYCARNLGPSFYFDYWGQGTLVTVSS | |
| 109 | EVQLVESGGGLVQPGGSLRLSCAASGFNIKDTYIHWVRQAPGKGLEW | Anti-HER2DIV V_{H} |
| | VARIYPTNGYTRYADSVKGRFTISADTSKNTAYLQMNSLRAEDTAVY | region |
| | YCSRWGGDGFYAMDYWGQGTLVTVSS | |
| 110 | DIQMTQSPSSLSASVGDRVTITCKASQDVSIGVAWYQQKPGKAPKLLI | Anti-HER2DII V_{L} |
| | YSASYRYTGVPSRFSGSGSGTDFTLTISSLQPEDFATYYCQQYYIYPYTF | region |
| | GQGTKVEIK | |
| 111 | DIQMTQSPSSLSASVGDRVTITCRASQDVNTAVAWYQQKPGKAPKLLI | Anti-HER2DIV V_{L} |
| | YSASFLYSGVPSRFSGSRSGTDFTLTISSLQPEDFATYYCQQHYTTPPTF | region |
| | GQGTKVEIK | |
| 112 | EVQLVESGGGLVQPGGSLRLSCAASGFTFTDYTMDWVRQAPGKCLE | Anti-HER2DII V_{H} |
| | WVADVNPNSGGSIYNQRFKGRFTLSVDRSKNTLYLQMNSLRAEDTAV | region_Gly to Cys |
| | YYCARNLGPSFYFDYWGQGTLVTVSS | |
| 113 | EVQLVESGGGLVQPGGSLRLSCAASGFNIKDTYIHWVRQAPGKCLEW | Anti-HER2DIV V_{H} |
| | VARIYPTNGYTRYADSVKGRFTISADTSKNTAYLQMNSLRAEDTAVY | region_Gly to Cys |
| | YCSRWGGDGFYAMDYWGQGTLVTVSS | |
| 114 | DIQMTQSPSSLSASVGDRVTITCKASQDVSIGVAWYQQKPGKAPKLLI | Anti-HER2DII V_{L} |
| | YSASYRYTGVPSRFSGSGSGTDFTLTISSLQPEDFATYYCQQYYIYPYTF | region_Gln to Cys |
| | GCGTKVEIK | |
| 115 | DIQMTQSPSSLSASVGDRVTITCRASQDVNTAVAWYQQKPGKAPKLLI | Anti-HER2DIV V_{L} |
| | YSASFLYSGVPSRFSGSRSGTDFTLTISSLQPEDFATYYCQQHYTTPPTF | region_Gln to Cys |
| | GCGTKVEIK | |
| 116 | GGSGGGSGGGSGGGSGGGSG (GGSG)5 | linker |
| 117 | GGGGS (G₄S) | linker |
| 118 | GGGGSGGGGS ((G₄S)₂) | linker |
| 119 | GGGGSGGGGSGGGGS ((G₄S)₃) | linker |
| 120 | GGGGSGGGGSGGGG ((G₄S)₂-G₄) | linker |
| 121 | GGGGSGGGGSGG | linker |
| 122 | GGGGGSGGGGS | linker |
| 123 | GGGGGSGGGGGSGGGGS | linker |
| 124 | GGGGSEPKSS | linker |
| 125 | ASTKGPSVF | linker |
| 126 | RTVAAPSVFI | linker |
| 127 | EPKSCDKTHTCPPCP | human IgG1 hinge |
| | | region |
| 128 | DKTHTCPPCP | Partial hinge region |
| 129 | EPKSSDKTHTCPPCP | Hinge region with Cys |
| | | to Ser mutation |
| 130 | APELLGGPSVFLFPPKPKDTLMISRTPEVTCVVVDVSHEDPEVKFNWY | Wild-type human Fc |
| | VDGVEVHNAKTKPREEQYNSTYRVVSVLTVLHQDWLNGKEYKCKVS | sequence |
| | NKALPAPIEKTISKAKGQPREPQVYTLPPSRDELTKNQVSLTCLVKGFY | positions 231-447 EU |
| | PSDIAVEWESNGQPENNYKTTPPVLDSDGSFFLYSKLTVDKSRWQQGN | index numbering |
| | VFSCSVMHEALHNHYTQKSLSLSPGK | |
| 131 | APELLGGPSVFLFPPKPKDTLMISRTPEVTCVVVDVSHEDPEVKFNWY | Fc sequence with knob |
| | VDGVEVHNAKTKPREEQYNSTYRVVSVLTVLHQDWLNGKEYKCKVS | mutation |
| | NKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQG | |
| | NVFSCSVMHEALHNHYTQKSLSLSPGK | |
| 132 | APEAAGGPSVFLFPPKPKDTLMISRTPEVTCVVVDVSHEDPEVKFNWY | Fc sequence with knob |
| | VDGVEVHNAKTKPREEQYNSTYRVVSVLTVLHQDWLNGKEYKCKVS | and LALA mutations |
| | NKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQG | |
| | NVFSCSVMHEALHNHYTQKSLSLSPGK | |
| 133 | APELLGGPSVFLFPPKPKDTLMISRTPEVTCVVVDVSHEDPEVKFNWY | Fc sequence with hole |
| | VDGVEVHNAKTKPREEQYNSTYRVVSVLTVLHQDWLNGKEYKCKVS | mutations |
| | NKALPAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGN | |
| | VFSCSVMHEALHNHYTQKSLSLSPGK | |
| 134 | APEAAGGPSVFLFPPKPKDTLMISRTPEVTCVVVDVSHEDPEVKFNWY | Fc sequence with hole |
| | VDGVEVHNAKTKPREEQYNSTYRVVSVLTVLHQDWLNGKEYKCKVS | and LALA mutations |
| | NKALPAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGN | |
| | VFSCSVMHEALHNHYTQKSLSLSPGK | |
| 135 | APELLGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGFY | |
| | PSDIAVEWESYGTEWSNYKTTPPVLDSDGSFFLYSKLTVSKEEWQQGF | |
| | VFSCSVMHEALHNHYTQKSLSLSPGK | |
| 136 | APELLGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob mutation |
| | NKALPAPIEKTISKAKGQPREPQVYTLPPSRDELTKNQVSLWCLVKGF | |
| | YPSDIAVEWESYGTEWSNYKTTPPVLDSDGSFFLYSKLTVSKEEWQQG | |
| | FVFSCSVMHEALHNHYTQKSLSLSPGK | |
| 137 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob and LALA |
| | NKALPAPIEKTISKAKGQPREPQVYTLPPSRDELTKNQVSLWCLVKGF | mutations |
| | YPSDIAVEWESYGTEWSNYKTTPPVLDSDGSFFLYSKLTVSKEEWQQG | |
| | FVFSCSVMHEALHNHYTQKSLSLSPGK | |
| 138 | APELLGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with hole mutations |
| | NKALPAPIEKTISKAKGQPREPQVYTLPPSRDELTKNQVSLSCAVKGFY | |
| | PSDIAVEWESYGTEWSNYKTTPPVLDSDGSFFLVSKLTVSKEEWQQGF | |
| | VFSCSVMHEALHNHYTQKSLSLSPGK | |
| 139 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with hole and LALA |
| | NKALPAPIEKTISKAKGQPREPQVYTLPPSRDELTKNQVSLSCAVKGFY | mutations |
| | PSDIAVEWESYGTEWSNYKTTPPVLDSDGSFFLVSKLTVSKEEWQQGF | |
| | VFSCSVMHEALHNHYTQKSLSLSPGK | |
| 140 | APELLGGPSVFLFPPKPKDTLMISRTPEVTCVVVDVSHEDPEVKFNWY | Clone CH3C.35.23.2 |
| | VDGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGFY | |
| | PSDIAVEWESYGTEWANYKTTPPVLDSDGSFFLYSKLTVTKEEWQQGF | |
| | VFSCSVMHEALHNHYTQKSLSLSPGK | |
| 141 | APELLGGPSVFLFPPKPKDTLMISRTPEVTCVVVDVSHEDPEVKFNWY | Clone CH3C.35.23.2 |
| | VDGVEVHNAKTKPREEQYNSTYRVVSVLTVLHQDWLNGKEYKCKVS | with knob mutation |
| | NKALPAPIEKTISKAKGQPREPQVYTLPPSRDELTKNQVSLWCLVKGF | |
| | YPSDIAVEWESYGTEWANYKTTPPVLDSDGSFFLYSKLTVTKEEWQQ | |
| | GFVFSCSVMHEALHNHYTQKSLSLSPGK | |
| 142 | APEAAGGPSVFLFPPKPKDTLMISRTPEVTCVVVDVSHEDPEVKFNWY | Clone CH3C.35.23.2 |
| | VDGVEVHNAKTKPREEQYNSTYRVVSVLTVLHQDWLNGKEYKCKVS | with knob and LALA |
| | NKALPAPIEKTISKAKGQPREPQVYTLPPSRDELTKNQVSLWCLVKGF | mutations |
| | YPSDIAVEWESYGTEWANYKTTPPVLDSDGSFFLYSKLTVTKEEWQQ | |
| | GFVFSCSVMHEALHNHYTQKSLSLSPGK | |
| 143 | APELLGGPSVFLFPPKPKDTLMISRTPEVTCVVVDVSHEDPEVKFNWY | Clone CH3C.35.23.2 |
| | VDGVEVHNAKTKPREEQYNSTYRVVSVLTVLHQDWLNGKEYKCKVS | with hole mutations |
| | NKALPAPIEKTISKAKGQPREPQVYTLPPSRDELTKNQVSLSCAVKGFY | |
| | PSDIAVEWESYGTEWANYKTTPPVLDSDGSFFLVSKLTVTKEEWQQGF | |
| | VFSCSVMHEALHNHYTQKSLSLSPGK | |
| 144 | APEAAGGPSVFLFPPKPKDTLMISRTPEVTCVVVDVSHEDPEVKFNWY | Clone CH3C.35.23.2 |
| | VDGVEVHNAKTKPREEQYNSTYRVVSVLTVLHQDWLNGKEYKCKVS | with hole and LALA |
| | NKALPAPIEKTISKAKGQPREPQVYTLPPSRDELTKNQVSLSCAVKGFY | mutations |
| | PSDIAVEWESYGTEWANYKTTPPVLDSDGSFFLVSKLTVTKEEWQQGF | |
| | VFSCSVMHEALHNHYTQKSLSLSPGK | |
| 145 | APELLGGPSVFLFPPKPKDTLMISRTPEVTCVVVDVSHEDPEVKFNWY | Clone CH3C.35.20.1 |
| | VDGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGFY | |
| | PSDIAVEWESFGTEWSSYKTTPPVLDSDGSFFLYSKLTVTKEEWQQGF | |
| | VFSCSVMHEALHNHYTQKSLSLSPGK | |
| 146 | APELLGGPSVFLFPPKPKDTLMISRTPEVTCVVVDVSHEDPEVKFNWY | Clone CH3C.35.20.1 |
| | VDGVEVHNAKTKPREEQYNSTYRVVSVLTVLHQDWLNGKEYKCKVS | with knob mutation |
| | NKALPAPIEKTISKAKGQPREPQVYTLPPSRDELTKNQVSLWCLVKGF | |
| | YPSDIAVEWESFGTEWSSYKTTPPVLDSDGSFFLYSKLTVTKEEWQQG | |
| | FVFSCSVMHEALHNHYTQKSLSLSPGK | |
| 147 | APEAAGGPSVFLFPPKPKDTLMISRTPEVTCVVVDVSHEDPEVKFNWY | Clone CH3C.35.20.1 |
| | VDGVEVHNAKTKPREEQYNSTYRVVSVLTVLHQDWLNGKEYKCKVS | with knob and LALA |
| | NKALPAPIEKTISKAKGQPREPQVYTLPPSRDELTKNQVSLWCLVKGF | mutations |
| | YPSDIAVEWESFGTEWSSYKTTPPVLDSDGSFFLYSKLTVTKEEWQQG | |
| | FVFSCSVMHEALHNHYTQKSLSLSPGK | |
| 148 | APELLGGPSVFLFPPKPKDTLMISRTPEVTCVVVDVSHEDPEVKFNWY | Clone CH3C.35.20.1 |
| | VDGVEVHNAKTKPREEQYNSTYRVVSVLTVLHQDWLNGKEYKCKVS | with hole mutations |
| | NKALPAPIEKTISKAKGQPREPQVYTLPPSRDELTKNQVSLSCAVKGFY | |
| | PSDIAVEWESFGTEWSSYKTTPPVLDSDGSFFLVSKLTVTKEEWQQGF | |
| | VFSCSVMHEALHNHYTQKSLSLSPGK | |
| 149 | APEAAGGPSVFLFPPKPKDTLMISRTPEVTCVVVDVSHEDPEVKFNWY | Clone CH3C.35.20.1 |
| | VDGVEVHNAKTKPREEQYNSTYRVVSVLTVLHQDWLNGKEYKCKVS | with hole and LALA |
| | NKALPAPIEKTISKAKGQPREPQVYTLPPSRDELTKNQVSLSCAVKGFY | mutations |
| | PSDIAVEWESFGTEWSSYKTTPPVLDSDGSFFLVSKLTVTKEEWQQGF | |
| | VFSCSVMHEALHNHYTQKSLSLSPGK | |
| 150 | MMDQARSAFSNLFGGEPLSYTRFSLARQVDGDNSHVEMKLAVDEEEN | Human transferrin |
| | ADNNTKANVTKPKRCSGSICYGTIAVIVFFLIGFMIGYLGYCKGVEPKT | receptor protein 1 |
| | ECERLAGTESPVREEPGEDFPAARRLYWDDLKRKLSEKLDSTDFTGTI | (TFR1) |
| | KLLNENSYVPREAGSQKDENLALYVENQFREFKLSKVWRDQHFVKIQ | |
| | VKDSAQNSVIIVDKNGRLVYLVENPGGYVAYSKAATVTGKLVHANFG | |
| | TKKDFEDLYTPVNGSIVIVRAGKITFAEKVANAESLNAIGVLIYMDQTK | |
| | FPIVNAELSFFGHAHLGTGDPYTPGFPSFNHTQFPPSRSSGLPNIPVQTIS | |
| | RAAAEKLFGNMEGDCPSDWKTDSTCRMVTSESKNVKLTVSNVLKEIK | |
| | ILNIFGVIKGFVEPDHYVVVGAQRDAWGPGAAKSGVGTALLLKLAQM | |
| | FSDMVLKDGFQPSRSIIFASWSAGDFGSVGATEWLEGYLSSLHLKAFT | |
| | YINLDKAVLGTSNFKVSASPLLYTLIEKTMQNVKHPVTGQFLYQDSNW | |
| | ASKVEKLTLDNAAFPFLAYSGIPAVSFCFCEDTDYPYLGTTMDTYKELI | |
| | ERIPELNKVARAAAEVAGQFVIKLTHD VELNLDYERYNSQLLSFVRDL | |
| | NQYRADIKEMGLSLQWLYSARGDFFRATSRLTTDFGNAEKTDRFVMK | |
| | KLNDRVMRVEYHFLSPYVSPKESPFRHVFWGSGSHTLPALLENLKLRK | |
| | QNNGAFNETLFRNQLALATWTIQGAANALSGDVWDIDNEF | |
| 151 | GGSG | Linker |
| 152 | GSGG | Linker |
| 153 | SGGG | Linker |
| 154 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | wild-type human Fc |
| | YCSRWGGDGFYAMDYWGQGTLVTVSSASTKGPSVFPLAPSSKSTSGG | sequence |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSCS | |
| | VMHEALHNHYTQKSLSLSPGK | |
| 155 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | wild-type Fc sequence |
| | YYCARNLGPSFYFDYWGQGTLVTVSSASTKGPSVFPLAPSSKSTSGGT | |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK | |
| 156 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole_v1 |
| | DIQMTQSPSSLSASVGDRVTITCKASQDVSIGVAWYQQKPGKAPKLLI | |
| | YSASYRYTGVPSRFSGSGSGTDFTLTISSLQPEDFATYYCQQYYIYPYTF | |
| | GQGTKVEIKGGGGSGGGGSEVQLVESGGGLVQPGGSLRLSCAASGFNI | |
| | KDTYIHWVRQAPGKGLEWVARIYPTNGYTRYADSVKGRFTISADTSK | |
| | NTAYLQMNSLRAEDTAVYYCSRWGGDGFYAMDYWGQGTLVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSRDE | |
| | LTKNQVSLSCAVKGFYPSDIAVEWESNGQPENNYKTTPPVLDSDGSFF | |
| | LVSKLTVDKSRWQQGNVFSCSVMHEALHNHYTQKSLSLSPGK | |
| 157 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole-anti-HER2DII |
| | YCSRWGGDGFYAMDYWGQGTLVTVSSASTKGPSVFPLAPSSKSTSGG | SCFv (V_{L}-V_{H} scFV) |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGSDIQMTQSPSSLSASVG | |
| | DRVTITCKASQDVSIGVAWYQQKPGKAPKLLIYSASYRYTGVPSRFSG | |
| | SGSGTDFTLTISSLQPEDFATYYCQQYYIYPYTFGQGTKVEIKGGSGGG | |
| | SGGGSGGGSGGGSGEVQLVESGGGLVQPGGSLRLSCAASGFTFTDYT | |
| | MDWVRQAPGKGLEWVADVNPNSGGSIYNQRFKGRFTLSVDRSKNTL | |
| | YLQMNSLRAEDTAVYYCARNLGPSFYFDYWGQGTLVTVSS | |
| 158 | EVQLVESGGGLVQPGGSLRLSCAASGFNIKDTYIHWVRQAPGKGLEW | Anti-HER2DIV Fab-Fc |
| | VARIYPTNGYTRYADSVKGRFTISADTSKNTAYLQMNSLRAEDTAVY | with hole-anti-HER2DII |
| | YCSRWGGDGFYAMDYWGQGTLVTVSSASTKGPSVFPLAPSSKSTSGG | V_{L} |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSC | |
| | SVMHEALHNHYTQKSLSLSPGGGGGGSGGGGGSGGGGSDIQMTQSPS | |
| | SLSASVGDRVTITCKASQDVSIGVAWYQQKPGKAPKLLIYSASYRYTG | |
| | VPSRFSGSGSGTDFTLTISSLQPEDFATYYCQQYYIYPYTFGQGTKVEIK | |
| 159 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGGGSGGGGSGGGGSEVQLVESGGGLVQPGGSLRLSCA | scFv)_v2 3(G4S) |
| | ASGFNIKDTYIHWVRQAPGKGLEWVARIYPTNGYTRYADSVKGRFTIS | |
| | ADTSKNTAYLQMNSLRAEDTAVYYCSRWGGDGFYAMDYWGQGTLV | |
| | TVSSGGGGSEPKSSDKTHTCPPCPAPELLGGPSVFLFPPKPKDTLMISRT | |
| | PEVTCVVVDVSHEDPEVKFNWYVDGVEVHNAKTKPREEQYNSTYRV | |
| | VSVLTVLHQDWLNGKEYKCKVSNKALPAPIEKTISKAKGQPREPQVYT | |
| | LPPSRDELTKNQVSLSCAVKGFYPSDIAVEWESNGQPE | |
| | NNYKTTPPVLDSDGSFFLVSKLTVDKSRWQQGNVFSCSVMHEALHNH | |
| | YTQKSLSLSPGK | |
| 160 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole v2 |
| | DIQMTQSPSSLSASVGDRVTITCKASQDVSIGVAWYQQKPGKAPKLLI | 5(GGSG)_(G4S) |
| | YSASYRYTGVPSRFSGSGSGTDFTLTISSLQPEDFATYYCQQYYIYPYTF | |
| | GQGTKVEIKGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLTVD | |
| | KSRWQQGNVFSCSVMHEALHNHYTQKSLSLSPGK | |
| 161 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGGGSGGGGSGGGGSDIQMT | with hole_v3 |
| | QSPSSLSASVGDRVTITCKASQDVSIGVAWYQQKPGKAPKLLIYSASYR | 3(G4S)_(G4S) |
| | YTGVPSRFSGSGSGTDFTLTISSLQPEDFATYYCQQYYIYPYTFGQGTK | |
| | VEIKGGGGSEVQLVESGGGLVQPGGSLRLSCAASGFNIKDTYIHWVRQ | |
| | APGKGLEWVARIYPTNGYTRYADSVKGRFTISADTSKNTAYLQMNSL | |
| | RAEDTAVYYCSRWGGDGFYAMDYWGQGTLVTVSSASTKGPSVFPLA | |
| | PSSKSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSR | |
| | WQQGNVFSCSVMHEALHNHYTQKSLSLSPGK | |
| 162 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGGGSGGGGSGGGGSDIQMT | with hole_v4 |
| | QSPSSLSASVGDRVTITCKASQDVSIGVAWYQQKPGKAPKLLIYSASYR | 3(G4S)_2(G4S) |
| | YTGVPSRFSGSGSGTDFTLTISSLQPEDFATYYCQQYYIYPYTFGQGTK | |
| | VEIKGGGGSGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLTVD | |
| | KSRWQQGNVFSCSVMHEALHNHYTQKSLSLSPGK | |
| 163 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA.kD |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPDVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 164 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA.kD.PG |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPDVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALG | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 165 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALAkD.PS |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPDVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALS | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 166 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA.PG |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALG | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 167 | EVQLVESGGGLVQPGGSLRLSCAASGFTFTDYTMDWVRQAPGKGLE | Anti-HER2DII Fab- |
| | WVADVNPNSGGSIYNQRFKGRFTLSVDRSKNTLYLQMNSLRAEDTAV | CH3C.35.23.4 with |
| | YYCARNLGPSFYFDYWGQGTLVTVSSASTKGPSVFPLAPSSKSTSGGT | knob LALA.PS |
| | AALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALS | |
| | APIEKTISKAKGQPREPQVYTLPPSRDELTKNQVSLWCLVKGFYPSDIA | |
| | VEWESYGTEWSNYKTTPPVLDSDGSFFLYSKLTVSKEEWQQGFVFSCS | |
| | VMHEALHNHYTQKSLSLSPGK | |
| 168 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA.kD |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPDVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKA | |
| | LPAPIEKTISKAKGQPREPQVYTLPPSRDELTKNQVSLWCLVKGFYPSD | |
| | IAVEWESYGTEWSNYKTTPPVLDSDGSFFLYSKLTVSKEEWQQGFVFS | |
| | CSVMHEALHNHYTQKSLSLSPGK | |
| 169 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA.kD.PG |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPDVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKA | |
| | LGAPIEKTISKAKGQPREPQVYTLPPSRDELTKNQVSLWCLVKGFYPSD | |
| | IAVEWESYGTEWSNYKTTPPVLDSDGSFFLYSKLTVSKEEWQQGFVFS | |
| | CSVMHEALHNHYTQKSLSLSPGK | |
| 170 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA.kD.PS |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPDVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKA | |
| | LSAPIEKTISKAKGQPREPQVYTLPPSRDELTKNQVSLWCLVKGFYPSD | |
| | IAVEWESYGTEWSNYKTTPPVLDSDGSFFLYSKLTVSKEEWQQGFVFS | |
| | CSVMHEALHNHYTQKSLSLSPGK | |
| 171 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA.PG |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | GAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGK | |
| 172 | EVQLVESGGGLVQPGGSLRLSCAASGFNIKDTYIHWVRQAPGKGLEW | Anti-HER2DIV Fab- |
| | VARIYPTNGYTRYADSVKGRFTISADTSKNTAYLQMNSLRAEDTAVY | CH3C.35.23.4 with |
| | YCSRWGGDGFYAMDYWGQGTLVTVSSASTKGPSVFPLAPSSKSTSGG | knob LALA.PS |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPEA | |
| | AGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | SAPIEKTISKAKGQPREPQVYTLPPSRDELTKNQVSLWCLVKGFYPSDI | |
| | AVEWESYGTEWSNYKTTPPVLDSDGSFFLYSKLTVSKEEWQQGFVFS | |
| | CSVMHEALHNHYTQKSLSLSPGK | |
| 173 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGSGGGSGGGSGGGSGGGSGEVQLVESGGGLVQPGGSL | scFv)_v1_hE |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSEPKSSDKTHTCPPCPAPELLGGPSVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPEEKTISKAKGQPR | |
| | EPQVYTLPPSRDELTKNQVSLSCAVKGFYPSDIAVEWESNGQPENNYK | |
| | TTPPVLDSDGSFFLVSKLTVDKSRWQQGNVFSCSVMHEALHNHYTQK | |
| | SLSLSPGK | |
| 174 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGSGGGSGGGSGGGSGGGSGEVQLVESGGGLVQPGGSL | scFv)_v1_hDE |
| | RLSCAASGFNIKDTYIHWVRQAPGKGLEWVARIYPTNGYTRYADSVK | |
| | GRFTISADTSKNTAYLQMNSLRAEDTAVYYCSRWGGDGFYAMDYWG | |
| | QGTLVTVSSGGGGSEPKSSDKTHTCPPCPAPELLGGPDVFLFPPKPKDT | |
| | LMISRTPEVTCVVVDVSHEDPEVKFNWYVDGVEVHNAKTKPREEQYN | |
| | STYRVVSVLTVLHQDWLNGKEYKCKVSNKALPAPEEKTISKAKGQPR | |
| | EPQVYTLPPSRDELTKNQVSLSCAVKGFYPSDIAVEWESNGQPENNYK | |
| | TTPPVLDSDGSFFLVSKLTVDKSRWQQGNVFSCSVMHEALHNHYTQK | |
| | SLSLSPGK | |
| 175 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGGGSGGGGSGGGGSEVQLVESGGGLVQPGGSLRLSCA | scFv)_v2 3(G4S)_hE |
| | ASGFNIKDTYIHWVRQAPGKGLEWVARIYPTNGYTRYADSVKGRFTIS | |
| | ADTSKNTAYLQMNSLRAEDTAVYYCSRWGGDGFYAMDYWGQGTLV | |
| | TVSSGGGGSEPKSSDKTHTCPPCPAPELLGGPSVFLFPPKPKDTLMISRT | |
| | PEVTCVVVDVSHEDPEVKFNWYVDGVEVHNAKTKPREEQYNSTYRV | |
| | VSVLTVLHQDWLNGKEYKCKVSNKALPAPEEKTISKAKGQPREPQVY | |
| | TLPPSRDELTKNQVSLSCAVKGFYPSDIAVEWESNGQPENNYKTTPPV | |
| | LDSDGSFFLVSKLTVDKSRWQQGNVFSCSVMHEALHNHYTQKSLSLS | |
| | PGK | |
| 176 | DIQMTQSPSSLSASVGDRVTITCRASQDVNTAVAWYQQKPGKAPKLLI | Anti-HER2DIV scFv- |
| | YSASFLYSGVPSRFSGSRSGTDFTLTISSLQPEDFATYYCQQHYTTPPTF | Fc with hole (V_{L}-V_{H} |
| | GQGTKVEIKGGGGSGGGGSGGGGSEVQLVESGGGLVQPGGSLRLSCA | scFv)_v2 3(G4S)_hDE |
| | ASGFNIKDTYIHWVRQAPGKGLEWVARIYPTNGYTRYADSVKGRFTIS | |
| | ADTSKNTAYLQMNSLRAEDTAVYYCSRWGGDGFYAMDYWGQGTLV | |
| | TVSSGGGGSEPKSSDKTHTCPPCPAPELLGGPDVFLFPPKPKDTLMISRT | |
| | PEVTCVVVDVSHEDPEVKFNWYVDGVEVHNAKTKPREEQYNSTYRV | |
| | VSVLTVLHQDWLNGKEYKCKVSNKALPAPEEKTISKAKGQPREPQVY | |
| | TLPPSRDELTKNQVSLSCAVKGFYPSDIAVEWESNGQPENNYKTTPPV | |
| | LDSDGSFFLVSKLTVDKSRWQQGNVFSCSVMHEALHNHYTQKSLSLS | |
| | PGK | |
| 177 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole_v1_hE |
| | DIQMTQSPSSLSASVGDRVTITCKASQDVSIGVAWYQQKPGKAPKLLI | |
| | YSASYRYTGVPSRFSGSGSGTDFTLTISSLQPEDFATYYCQQYYIYPYTF | |
| | GQGTKVEIKGGGGSGGGGSEVQLVESGGGLVQPGGSLRLSCAASGFNI | |
| | KDTYIHWVRQAPGKGLEWVARIYPTNGYTRYADSVKGRFTISADTSK | |
| | NTAYLQMNSLRAEDTAVYYCSRWGGDGFYAMDYWGQGTLVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPEEKTISKAKGQPREPQVYTLPPSRDE | |
| | LTKNQVSLSCAVKGFYPSDIAVEWESNGQPENNYKTTPPVLDSDGSFF | |
| | LVSKLTVDKSRWQQGNVFSCSVMHEALHNHYTQKSLSLSPGK | |
| 178 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole_v1_hDE |
| | DIQMTQSPSSLSASVGDRVTITCKASQDVSIGVAWYQQKPGKAPKLLI | |
| | YSASYRYTGVPSRFSGSGSGTDFTLTISSLQPEDFATYYCQQYYIYPYTF | |
| | GQGTKVEIKGGGGSGGGGSEVQLVESGGGLVQPGGSLRLSCAASGFNI | |
| | KDTYIHWVRQAPGKGLEWVARIYPTNGYTRYADSVKGRFTISADTSK | |
| | NTAYLQMNSLRAEDTAVYYCSRWGGDGFYAMDYWGQGTLVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPELLGGPDVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPEEKTISKAKGQPREPQVYTLPPSRDE | |
| | LTKNQVSLSCAVKGFYPSDIAVEWESNGQPENNYKTTPPVLDSDGSFF | |
| | LVSKLTVDKSRWQQGNVFSCSVMHEALHNHYTQKSLSLSPGK | |
| 179 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole_v2 |
| | DIQMTQSPSSLSASVGDRVTITCKASQDVSIGVAWYQQKPGKAPKLLI | 5(GGSG)_(G4S)_hE |
| | YSASYRYTGVPSRFSGSGSGTDFTLTISSLQPEDFATYYCQQYYIYPYTF | |
| | GQGTKVEIKGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPEEKTISKAKGQPREPQVYTLPPSRDELTKNQVS | |
| | LSCAVKGFYPSDIAVEWESNGQPENNYKTTPPVLDSDGSFFLVSKLTV | |
| | DKSRWQQGNVFSCSVMHEALHNHYTQKSLSLSPGK | |
| 180 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGSGGGSGGGSGGGSGGGSG | with hole v2 |
| | DIQMTQSPSSLSASVGDRVTITCKASQDVSIGVAWYQQKPGKAPKLLI | 5(GGSG)_(G4S)_hDE |
| | YSASYRYTGVPSRFSGSGSGTDFTLTISSLQPEDFATYYCQQYYIYPYTF | |
| | GQGTKVEIKGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPDVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPEEKTISKAKGQPREPQVYTLPPSRDELTKNQVS | |
| | LSCAVKGFYPSDIAVEWESNGQPENNYKTTPPVLDSDGSFFLVSKLTV | |
| | DKSRWQQGNVFSCSVMHEALHNHYTQKSLSLSPGK | |
| 181 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGGGSGGGGSGGGGSDIQMT | with hole v4 |
| | QSPSSLSASVGDRVTITCKASQDVSIGVAWYQQKPGKAPKLLIYSASYR | 3(G4S)_2(G4S)_hE |
| | YTGVPSRFSGSGSGTDFTLTISSLQPEDFATYYCQQYYIYPYTFGQGTK | |
| | VEIKGGGGSGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPEEKTISKAKGQPREPQVYTLPPSRDELTKNQVS | |
| | LSCAVKGFYPSDIAVEWESNGQPENNYKTTPPVLDSDGSFFLVSKLTV | |
| | DKSRWQQGNVFSCSVMHEALHNHYTQKSLSLSPGK | |
| 182 | EVQLVESGGGLVQPGGSLRLSCAASGFTFTDYTMDWVRQAPGKGLE | Anti-HER2DII scFv- |
| | WVADVNPNSGGSIYNQRFKGRFTLSVDRSKNTLYLQMNSLRAEDTAV | anti-HER2DIV Fab-Fc |
| | YYCARNLGPSFYFDYWGQGTLVTVSSGGGGSGGGGSGGGGSDIQMT | with hole v4 |
| | QSPSSLSASVGDRVTITCKASQDVSIGVAWYQQKPGKAPKLLIYSASYR | 3(G4S)_2(G4S)_hDE |
| | YTGVPSRFSGSGSGTDFTLTISSLQPEDFATYYCQQYYIYPYTFGQGTK | |
| | VEIKGGGGSGGGGSEVQLVESGGGLVQPGGSLRLSCAASGFNIKDTYI | |
| | HWVRQAPGKGLEWVARIYPTNGYTRYADSVKGRFTISADTSKNTAYL | |
| | QMNSLRAEDTAVYYCSRWGGDGFYAMDYWGQGTLVTVSSASTKGPS | |
| | VFPLAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPKSCD | |
| | KTHTCPPCPAPELLGGPDVFLFPPKPKDTLMISRTPEVTCVVVDVSHED | |
| | PEVKFNWYVDGVEVHNAKTKPREEQYNSTYRVVSVLTVLHQDWLNG | |
| | KEYKCKVSNKALPAPEEKTISKAKGQPREPQVYTLPPSRDELTKNQVS | |
| | LSCAVKGFYPSDIAVEWESNGQPENNYKTTPPVLDSDGSFFLVSKLTV | |
| | DKSRWQQGNVFSCSVMHEALHNHYTQKSLSLSPGK | |
| 183 | APELLGGPDVFLFPPKPKDTLMISRTPEVTCVVVDVSHEDPEVKFNWY | Fc sequence with hole |
| | VDGVEVHNAKTKPREEQYNSTYRVVSVLTVLHQDWLNGKEYKCKVS | mutations.hD |
| | NKALPAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGN | |
| | VFSCSVMHEALHNHYTQKSLSLSPGK | |
| 184 | APELLGGPSVFLFPPKPKDTLMISRTPEVTCVVVDVSHEDPEVKFNWY | Fc sequence with hole |
| | VDGVEVHNAKTKPREEQYNSTYRVVSVLTVLHQDWLNGKEYKCKVS | mutations.hE |
| | NKALPAPEEKTISKAKGQPREPQVYTLPPSRDELTKNQVSLSCAVKGF | |
| | YPSDIAVEWESNGQPENNYKTTPPVLDSDGSFFLVSKLTVDKSRWQQG | |
| | NVFSCSVMHEALHNHYTQKSLSLSPGK | |
| 185 | APELLGGPDVFLFPPKPKDTLMISRTPEVTCVVVDVSHEDPEVKFNWY | Fc sequence with hole |
| | VDGVEVHNAKTKPREEQYNSTYRVVSVLTVLHQDWLNGKEYKCKVS | mutations.hDE |
| | NKALPAPEEKTISKAKGQPREPQVYTLPPSRDELTKNQVSLSCAVKGF | |
| | YPSDIAVEWESNGQPENNYKTTPPVLDSDGSFFLVSKLTVDKSRWQQG | |
| | NVFSCSVMHEALHNHYTQKSLSLSPGK | |
| 186 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, and |
| | NKALPAPIEKTISKAKGQPREPQVYTLPPSRDELTKNQVSLWCLVKGF | kD |
| | YPSDIAVEWESYGTEWSNYKTTPPVLDSDGSFFLYSKLTVSKEEWQQG | |
| | FVFSCSVMHEALHNHYTQKSLSLSPGK | |
| 187 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, and |
| | NKALPAPEEKTISKAKGQPREPQVYTLPPSRDELTKNQVSLWCLVKGF | kE |
| | YPSDIAVEWESYGTEWSNYKTTPPVLDSDGSFFLYSKLTVSKEEWQQG | |
| | FVFSCSVMHEALHNHYTQKSLSLSPGK | |
| 188 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, and |
| | NKALPAPEEKTISKAKGQPREPQVYTLPPSRDELTKNQVSLWCLVKGF | kDE |
| | YPSDIAVEWESYGTEWSNYKTTPPVLDSDGSFFLYSKLTVSKEEWQQG | |
| | FVFSCSVMHEALHNHYTQKSLSLSPGK | |
| 189 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, and |
| | NKALGAPIEKTISKAKGQPREPQVYTLPPSRDELTKNQVSLWCLVKGF | PG |
| | YPSDIAVEWESYGTEWSNYKTTPPVLDSDGSFFLYSKLTVSKEEWQQG | |
| | FVFSCSVMHEALHNHYTQKSLSLSPGK | |
| 190 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, and |
| | NKALSAPIEKTISKAKGQPREPQVYTLPPSRDELTKNQVSLWCLVKGF | PS |
| | YPSDIAVEWESYGTEWSNYKTTPPVLDSDGSFFLYSKLTVSKEEWQQG | |
| | FVFSCSVMHEALHNHYTQKSLSLSPGK | |
| 191 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PG, |
| | NKALGAPIEKTISKAKGQPREPQVYTLPPSRDELTKNQVSLWCLVKGF | and kD |
| | YPSDIAVEWESYGTEWSNYKTTPPVLDSDGSFFLYSKLTVSKEEWQQG | |
| | FVFSCSVMHEALHNHYTQKSLSLSPGK | |
| 192 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PS, |
| | NKALSAPIEKTISKAKGQPREPQVYTLPPSRDELTKNQVSLWCLVKGF | and kD |
| | YPSDIAVEWESYGTEWSNYKTTPPVLDSDGSFFLYSKLTVSKEEWQQG | |
| | FVFSCSVMHEALHNHYTQKSLSLSPGK | |
| 193 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PG, |
| | NKALGAPEEKTISKAKGQPREPQVYTLPPSRDELTKNQVSLWCLVKGF | and kE |
| | YPSDIAVEWESYGTEWSNYKTTPPVLDSDGSFFLYSKLTVSKEEWQQG | |
| | FVFSCSVMHEALHNHYTQKSLSLSPGK | |
| 194 | APEAAGGPSVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PS, |
| | NKALSAPEEKTISKAKGQPREPQVYTLPPSRDELTKNQVSLWCLVKGF | and kE |
| | YPSDIAVEWESYGTEWSNYKTTPPVLDSDGSFFLYSKLTVSKEEWQQG | |
| | FVFSCSVMHEALHNHYTQKSLSLSPGK | |
| 195 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PG, |
| | NKALGAPEEKTISKAKGQPREPQVYTLPPSRDELTKNQVSLWCLVKGF | and kDE |
| | YPSDIAVEWESYGTEWSNYKTTPPVLDSDGSFFLYSKLTVSKEEWQQG | |
| | FVFSCSVMHEALHNHYTQKSLSLSPGK | |
| 196 | APEAAGGPDVFLFPPKPKDTLMISRTPEVTCVVVDVSHEDPEVKFNWY | Clone CH3C.35.23.4 |
| | VDGVEVHNAKTKPREEQYNSTYRVVSVLTVLHQDWLNGKEYKCKVS | with knob, LALA, PS, |
| | NKALSAPEEKTISKAKGQPREPQVYTLPPSRDELTKNQVSLWCLVKGF | and kDE |
| | YPSDIAVEWESYGTEWSNYKTTPPVLDSDGSFFLYSKLTVSKEEWQQG | |
| | FVFSCSVMHEALHNHYTQKSLSLSPGK | |

What is claimed is:

1. A protein comprising:
(a) a first Fc polypeptide that is fused at the N-terminus to an Fd portion of a Fab;
(b) a second Fc polypeptide that is fused at the N-terminus to a single-chain variable fragment (scFv), wherein the first and second Fc polypeptides form an Fc dimer; and
(c) a light chain polypeptide that pairs with the Fd portion recited in (a) to form a Fab,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.

2. The protein of claim 1, wherein the first Fc polypeptide and/or the second Fc polypeptide independently comprising the S239D and/or the I332E substitution is capable of enhancing HER2-mediated effector function.

3. The protein of claim 1, wherein:
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.

4. The protein of claim 3, wherein:
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.

5. The protein of claim 4, wherein:
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at 332.

6. The protein of claim 1, wherein the Fab binds to subdomain II of human HER2 and the scFv binds to subdomain IV of human HER2.

7. The protein of claim 1, wherein the Fab binds to subdomain IV of human HER2 and the scFv binds to subdomain II of human HER2.

8. The protein of claim 1, wherein the second Fc polypeptide is fused to the scFv via a first linker.

9. The protein of claim 8, wherein the first linker has a length from 1 to 20 amino acids.

10. The protein of claim 8, wherein the first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

11. The protein of claim 1, wherein the scFv comprises a V_{L} region and a V_{H} region that are connected via a second linker.

12. The protein of claim 11, wherein the second linker has a length from 1 to 20 amino acids.

13. The protein of claim 11, wherein the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

14. The protein of claim 1, wherein the first Fc polypeptide and/or the second Fc polypeptide specifically binds to a transferrin receptor (TfR).

15. The protein of claim 1, wherein the first Fc polypeptide and the second Fc polypeptide each comprises modifications that promote heterodimerization.

16. The protein of claim 15, wherein the first Fc polypeptide comprises a T366W substitution and the second Fc polypeptide comprises T366S, L368A, and Y407V substitutions, according to EU numbering.

17. The protein of claim 15, wherein the first Fc polypeptide comprises T366S, L368A, and Y407V substitutions and the second Fc polypeptide comprises a T366W substitution, according to EU numbering.

18. The protein of claim 14, wherein the first Fc polypeptide and/or the second Fc polypeptide independently comprises modifications that reduce TfR-mediated effector function.

19. The protein of claim 18, wherein the modifications that reduce effector function are L234A and L235A substitutions, according to EU numbering.

20. The protein of claim 19, wherein the first Fc polypeptide specifically binds to TfR and comprises L234A and L235A substitutions.

21. The protein of claim 20, wherein the first Fc polypeptide further comprises a P329G or a P329S substitution, according to EU numbering.

22. The protein of claim 21, wherein the second Fc polypeptide comprises Leu at positions 234 and 235 and a proline at position 329, according to EU numbering.

23. The protein of claim 19, wherein the second Fc polypeptide specifically binds to TfR and comprises L234A and L235A substitutions.

24. The protein of claim 23, wherein the second Fc polypeptide further comprises a P329G or a P329S substitution, according to EU numbering.

25. The protein of claim 24, wherein the first Fc polypeptide comprises Leu at positions 234 and 235 and a proline at position 329, according to EU numbering.

26. The protein of claim 1, wherein a hinge region or a portion thereof is linked to the N-terminus of the first Fc polypeptide and/or the second Fc polypeptide.

27. The protein of claim 1, wherein the first Fc polypeptide and/or the second Fc polypeptide independently comprises a sequence having at least 90% identity to a sequence selected from the group consisting of SEQ ID NOS:131-149 and 183-196.

28. The protein of claim 27, wherein the first Fc polypeptide or the second Fc polypeptide comprises a sequence having at least 90% identity to a sequence selected from SEQ ID NOS:133 and 183-185.

29. The protein of claim 27, wherein the first Fc polypeptide or the second Fc polypeptide comprises a sequence having at least 90% identity to a sequence selected from SEQ ID NOS:137 and 186-196.

30. The protein of claim 1, wherein the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to a sequence selected from SEQ ID NOS:135-139 and 186-196.

31. The protein of claim 1, wherein the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137, and
the second Fc polypeptide comprises Ser at position 366, Ala at position 368, and Val at position 407, according to EU numbering, and a sequence having at least 90% identity to the sequence of SEQ ID NO:133.

32. The protein of claim 1, wherein the first Fc polypeptide comprises Ser at position 366, Ala at position 368, and Val at position 407, according to EU numbering, and a sequence having at least 90% identity to the sequence of SEQ ID NO:133, and
the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137.

33. A protein comprising:
(a) (i) a first heavy chain polypeptide comprising a sequence having at least 90% identity to the sequence of SEQ ID NO:1, (ii) a second heavy chain polypeptide comprising a sequence having at least 90% identity to the sequence of SEQ ID NO:159, and (iii) a light chain polypeptide comprising a sequence having at least 90% identity to the sequence of SEQ ID NO:25; or
(b) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(c) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:174, and (iii) a light chain polypeptide comprising a sequence having at least 90% identity to the sequence of SEQ ID NO:25; or
(d) (i) a first heavy chain polypeptide comprising a sequence having at least 90% identity to the sequence of SEQ ID NO: 166, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(e) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(f) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:174, and (iii) a light chain polypeptide comprising a sequence having at least 90% identity to the sequence of SEQ ID NO:25; or
(g) (i) a first heavy chain polypeptide comprising a sequence having at least 90% identity to the sequence of SEQ ID NO: 167, (ii) a second heavy chain polypeptide comprising a sequence having at least 90% identity to the sequence of SEQ ID NO:173, and (iii) a light chain polypeptide comprising a sequence having at least 90% identity to the sequence of SEQ ID NO:25; or
(h) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(i) (i) a first heavy chain polypeptide comprising a sequence having at least 90% identity to the sequence of SEQ ID NO: 164, (ii) a second heavy chain polypeptide comprising a sequence having at least 90% identity to the sequence of SEQ ID NO:176, and (iii) a light chain polypeptide comprising a sequence having at least 90% identity to the sequence of SEQ ID NO:25; or
(j) (i) a first heavy chain polypeptide comprising a sequence having at least 90% identity to the sequence of SEQ ID NO: 166, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(k) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25; or
(l) (i) a first heavy chain polypeptide comprising a sequence having at least 90% identity to the sequence of SEQ ID NO: 165, (ii) a second heavy chain polypeptide comprising a sequence having at least 90% identity to the sequence of SEQ ID NO:176, and (iii) a light chain polypeptide comprising a sequence having at least 90% identity to the sequence of SEQ ID NO:25; or
(m) (i) a first heavy chain polypeptide comprising a sequence having at least 90% identity to the sequence of SEQ ID NO: 167, (ii) a second heavy chain polypeptide comprising a sequence having at least 90% identity to the sequence of SEQ ID NO:175, and (iii) a light chain polypeptide comprising a sequence having at least 90% identity to the sequence of SEQ ID NO:25.

34. The protein of claim 33, wherein:
(a) (i) the first heavy chain polypeptide comprises the sequence of SEQ ID NO:1, (ii) the second heavy chain polypeptide comprises the sequence of SEQ ID NO:159, and (iii) the light chain polypeptide comprises the sequence of SEQ ID NO:25; or
(b) (i) the first heavy chain polypeptide comprises the sequence of SEQ ID NO:164, (ii) the second heavy chain polypeptide comprises the sequence of SEQ ID NO:173, and (iii) the light chain polypeptide comprises the sequence of SEQ ID NO:25; or
(c) (i) the first heavy chain polypeptide comprises the sequence of SEQ ID NO:164, (ii) the second heavy chain polypeptide comprises the sequence of SEQ ID NO:174, and (iii) the light chain polypeptide comprises the sequence of SEQ ID NO:25; or
(d) (i) the first heavy chain polypeptide comprises the sequence of SEQ ID NO:166, (ii) the second heavy chain polypeptide comprises the sequence of SEQ ID NO:173, and (iii) the light chain polypeptide comprises the sequence of SEQ ID NO:25; or
(e) (i) the first heavy chain polypeptide comprises the sequence of SEQ ID NO:165, (ii) the second heavy chain polypeptide comprises the sequence of SEQ ID NO: 173, and (iii) the light chain polypeptide comprises the sequence of SEQ ID NO:25; or
(f) (i) the first heavy chain polypeptide comprises the sequence of SEQ ID NO:165, (ii) the second heavy chain polypeptide comprises the sequence of SEQ ID NO:174, and (iii) the light chain polypeptide comprises the sequence of SEQ ID NO:25; or
(g) (i) the first heavy chain polypeptide comprises the sequence of SEQ ID NO:167, (ii) the second heavy chain polypeptide comprises the sequence of SEQ ID NO:173, and (iii) the light chain polypeptide comprises the sequence of SEQ ID NO:25; or
(h) (i) the first heavy chain polypeptide comprises the sequence of SEQ ID NO:164, (ii) the second heavy chain polypeptide comprises the sequence of SEQ ID NO:175, and (iii) the light chain polypeptide comprises the sequence of SEQ ID NO:25; or
(i) (i) the first heavy chain polypeptide comprises the sequence of SEQ ID NO:164, (ii) the second heavy chain polypeptide comprises the sequence of SEQ ID NO:176, and (iii) the light chain polypeptide comprises the sequence of SEQ ID NO:25; or
(j) (i) the first heavy chain polypeptide comprises the sequence of SEQ ID NO:166, (ii) the second heavy chain polypeptide comprises the sequence of SEQ ID NO:175, and (iii) the light chain polypeptide comprises the sequence of SEQ ID NO:25; or
(k) (i) the first heavy chain polypeptide comprises the sequence of SEQ ID NO:165, (ii) the second heavy chain polypeptide comprises the sequence of SEQ ID NO:175, and (iii) the light chain polypeptide comprises the sequence of SEQ ID NO:25; or
(l) (i) the first heavy chain polypeptide comprises the sequence of SEQ ID NO:165, (ii) the second heavy chain polypeptide comprises the sequence of SEQ ID NO:176, and (iii) the light chain polypeptide comprises the sequence of SEQ ID NO:25; or
(m) (i) the first heavy chain polypeptide comprises the sequence of SEQ ID NO:167, (ii) the second heavy chain polypeptide comprises the sequence of SEQ ID NO:175, and (iii) the light chain polypeptide comprises the sequence of SEQ ID NO:25.

35. A protein comprising:
(a) a first Fc polypeptide that is fused at the N-terminus to an Fd portion of a Fab;
(b) a second Fc polypeptide that is fused at the N-terminus to an Fd portion of a Fab, wherein the first and second Fc polypeptides form an Fc dimer; and
(c) two light chain polypeptides that each pairs with each Fd portion recited in (a) and (b) to form a Fab,
wherein the Fd portion in (a) and/or (b) is fused at the N-terminus to an scFv,
wherein the Fab binds to subdomain II of human HER2 and the scFv binds to subdomain IV of human HER2, or wherein the Fab binds to subdomain IV of human HER2 and the scFv binds to subdomain II of human HER2, and
wherein the first Fc polypeptide and/or the second Fc polypeptide independently comprises a S239D and/or a I332E substitution, according to EU numbering.

36. The protein of claim 35, wherein the first Fc polypeptide and/or the second Fc polypeptide independently comprising the S239D and/or the I332E substitution is capable of enhancing HER2-mediated effector function.

37. The protein of claim 35, wherein:
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.

38. The protein of claim 37, wherein:
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.

39. The protein of claim 38, wherein:
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at position 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at position 332.

40. The protein of claim 35, wherein the Fab binds to subdomain II of human HER2 and the scFv binds to subdomain IV of human HER2.

41. The protein of claim 35, wherein the Fab binds to subdomain IV of human HER2 and the scFv binds to subdomain II of human HER2.

42. The protein of claim 35, wherein the Fd portion in (a) or (b) is fused at the N-terminus to the scFv.

43. The protein of claim 35, wherein the Fd portion in (a) and/or (b) is fused to the scFv via a first linker.

44. The protein of claim 43, wherein the first linker has a length from 1 to 20 amino acids.

45. The protein of claim 43, wherein the first linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

46. The protein of claim 35, wherein the scFv comprises a V_{L} region and a V_{H} region that are connected via a second linker.

47. The protein of claim 46, wherein the second linker has a length from 1 to 20 amino acids.

48. The protein of claim 46, wherein the second linker comprises a sequence of any one of GGSGGGSGGGSGGGSGGGSG (SEQ ID NO:116; (GGSG)₅), GGGGS (SEQ ID NO:117; G₄S), GGGGSGGGGS (SEQ ID NO:118; (G₄S)₂), GGGGSGGGGSGGGGS (SEQ ID NO:119; (G₄S)₃), GGGGSGGGGSGGGG (SEQ ID NO:120; (G₄S)₂-G₄), GGGGSGGGGSGG (SEQ ID NO:121), GGGGGSGGGGS (SEQ ID NO:122), and GGGGGSGGGGGSGGGGS (SEQ ID NO:123).

49. The protein of claim 35, wherein the first Fc polypeptide and/or the second Fc polypeptide specifically binds to a transferrin receptor (TfR).

50. The protein of claim 35, wherein the first Fc polypeptide and the second Fc polypeptide each comprises modifications that promote heterodimerization.

51. The protein of claim 50, wherein the first Fc polypeptide comprises a T366W substitution and the second Fc polypeptide comprises T366S, L368A, and Y407V substitutions, according to EU numbering.

52. The protein of claim 50, wherein the first Fc polypeptide comprises T366S, L368A, and Y407V substitutions and the second Fc polypeptide comprises a T366W substitution, according to EU numbering.

53. The protein of claim 49, wherein the first Fc polypeptide and/or the second Fc polypeptide independently comprises modifications that reduce TfR-mediated effector function.

54. The protein of claim 53, wherein the modifications that reduce effector function are L234A and L235A substitutions, according to EU numbering.

55. The protein of claim 54, wherein the first Fc polypeptide specifically binds to TfR and comprises L234A and L235A substitutions.

56. The protein of claim 55, wherein the first Fc polypeptide further comprises a P329G or a P329S substitution, according to EU numbering.

57. The protein of claim 56, wherein the second Fc polypeptide comprises Leu at positions 234 and 235 and proline at position 329, according to EU numbering.

58. The protein of claim 54, wherein the second Fc polypeptide specifically binds to TfR and comprises L234A and L235A substitutions.

59. The protein of claim 58, wherein the second Fc polypeptide further comprises a P329G or a P329S substitution, according to EU numbering.

60. The protein of claim 59, wherein the first Fc polypeptide comprises Leu at positions 234 and 235 and proline at position 329, according to EU numbering.

61. The protein of claim 35, wherein a hinge region or a portion thereof is linked to the N-terminus of the first Fc polypeptide and/or the second Fc polypeptide.

62. The protein of claim 35, wherein the first Fc polypeptide and/or the second Fc polypeptide independently comprises a sequence having at least 90% identity to a sequence selected from the group consisting of SEQ ID NOS:131-149 and 183-196.

63. The protein of claim 62, wherein the first Fc polypeptide or the second Fc polypeptide comprises a sequence having at least 90% identity to a sequence selected from SEQ ID NOS:133 and 183-185.

64. The protein of claim 62, wherein the first Fc polypeptide or the second Fc polypeptide comprises a sequence having at least 90% identity to a sequence selected from SEQ ID NOS:137 and 186-196.

65. The protein of claim 35, wherein the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to a sequence selected from SEQ ID NOS:135-139 and 186-196.

66. The protein of claim 35, wherein the first Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137, and
the second Fc polypeptide comprises Ser at position 366, Ala at position 368, and Val at position 407, according to EU numbering, and a sequence having at least 90% identity to the sequence of SEQ ID NO:133.

67. The protein of claim 35, wherein the first Fc polypeptide comprises Ser at position 366, Ala at position 368, and Val at position 407, according to EU numbering, and a sequence having at least 90% identity to the sequence of SEQ ID NO:133, and
the second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:137.

68. A protein comprising:
(a) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:160, and (iii) two light chain polypeptides each comprising a sequence having at least 90% identity to the sequence of SEQ ID NO:26; or
(b) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:161, and (iii) two light chain polypeptides each comprising a sequence having at least 90% identity to the sequence of SEQ ID NO:26; or
(c) (i) a first heavy chain polypeptide comprising a sequence having at least 90% identity to the sequence of SEQ ID NO:6, (ii) a second heavy chain polypeptide comprising a sequence having at least 90% identity to the sequence of SEQ ID NO:162, and (iii) two light chain polypeptides each comprising a sequence having at least 90% identity to the sequence of SEQ ID NO:26; or
(d) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(e) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:178, and (iii) two light chain polypeptides each comprising a sequence having at least 90% identity to the sequence of SEQ ID NO:26; or
(f) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(g) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(h) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:178, and (iii) two light chain polypeptides each comprising a sequence having at least 90% identity to the sequence of SEQ ID NO:26; or
(i) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:177, and (iii) two light chain polypeptides each comprising a sequence having at least 90% identity to the sequence of SEQ ID NO:26; or
(j) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(k) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:180, and (iii) two light chain polypeptides each comprising a sequence having at least 90% identity to the sequence of SEQ ID NO:26; or
(l) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(m) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(n) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:180, and (iii) two light chain polypeptides each comprising a sequence having at least 90% identity to the sequence of SEQ ID NO:26; or
(o) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:179, and (iii) two light chain polypeptides each comprising a sequence having at least 90% identity to the sequence of SEQ ID NO:26; or
(p) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(q) (i) a first heavy chain polypeptide comprising a sequence having at least 90% identity to the sequence of SEQ ID NO: 169, (ii) a second heavy chain polypeptide comprising a sequence having at least 90% identity to the sequence of SEQ ID NO:182, and (iii) two light chain polypeptides each comprising a sequence having at least 90% identity to the sequence of SEQ ID NO:26; or
(r) (i) a first heavy chain polypeptide comprising a sequence having at least 90% identity to the sequence of SEQ ID NO: 171, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(s) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26; or
(t) (i) a first heavy chain polypeptide comprising a sequence having at least 90% identity to the sequence of SEQ ID NO: 170, (ii) a second heavy chain polypeptide comprising a sequence having at least 90% identity to the sequence of SEQ ID NO:182, and (iii) two light chain polypeptides each comprising a sequence having at least 90% identity to the sequence of SEQ ID NO:26; or
(u) (i) a first heavy chain polypeptide comprising a sequence having at least 90% identity to the sequence of SEQ ID NO: 172, (ii) a second heavy chain polypeptide comprising a sequence having at least 90% identity to the sequence of SEQ ID NO:181, and (iii) two light chain polypeptides each comprising a sequence having at least 90% identity to the sequence of SEQ ID NO:26.

69. The protein of claim 68, wherein:
(a) (i) the first heavy chain polypeptide comprises the sequence of SEQ ID NO:6, (ii) the second heavy chain polypeptide comprises the sequence of SEQ ID NO:160, and (iii) the two light chain polypeptides each comprises the sequence of SEQ ID NO:26; or
(b) (i) the first heavy chain polypeptide comprises the sequence of SEQ ID NO:6, (ii) the second heavy chain polypeptide comprises the sequence of SEQ ID NO:161, and (iii) the two light chain polypeptides each comprises the sequence of SEQ ID NO:26; or
(c) (i) the first heavy chain polypeptide comprises the sequence of SEQ ID NO:6, (ii) the second heavy chain polypeptide comprises the sequence of SEQ ID NO: 162, and (iii) the two light chain polypeptides each comprises the sequence of SEQ ID NO:26; or
(d) (i) the first heavy chain polypeptide comprises the sequence of SEQ ID NO:169, (ii) the second heavy chain polypeptide comprises the sequence of SEQ ID NO:177, and (iii) the two light chain polypeptides each comprises the sequence of SEQ ID NO:26; or
(e) (i) the first heavy chain polypeptide comprises the sequence of SEQ ID NO:169, (ii) the second heavy chain polypeptide comprises the sequence of SEQ ID NO:178, and (iii) the two light chain polypeptides each comprises the sequence of SEQ ID NO:26; or
(f) (i) the first heavy chain polypeptide comprises the sequence of SEQ ID NO:171, (ii) the second heavy chain polypeptide comprises the sequence of SEQ ID NO:177, and (iii) the two light chain polypeptides each comprises the sequence of SEQ ID NO:26; or
(g) (i) the first heavy chain polypeptide comprises the sequence of SEQ ID NO:170, (ii) the second heavy chain polypeptide comprises the sequence of SEQ ID NO:177, and (iii) the two light chain polypeptides each comprises the sequence of SEQ ID NO:26; or
(h) (i) the first heavy chain polypeptide comprises the sequence of SEQ ID NO:170, (ii) the second heavy chain polypeptide comprises the sequence of SEQ ID NO:178, and (iii) the two light chain polypeptides each comprises the sequence of SEQ ID NO:26; or
(i) (i) the first heavy chain polypeptide comprises the sequence of SEQ ID NO:172, (ii) the second heavy chain polypeptide comprises the sequence of SEQ ID NO:177, and (iii) the two light chain polypeptides each comprises the sequence of SEQ ID NO:26; or
(j) (i) the first heavy chain polypeptide comprises the sequence of SEQ ID NO:169, (ii) the second heavy chain polypeptide comprises the sequence of SEQ ID NO:179, and (iii) the two light chain polypeptides each comprises the sequence of SEQ ID NO:26; or
(k) (i) the first heavy chain polypeptide comprises the sequence of SEQ ID NO:169, (ii) the second heavy chain polypeptide comprises the sequence of SEQ ID NO:180, and (iii) the two light chain polypeptides each comprises the sequence of SEQ ID NO:26; or
(l) (i) the first heavy chain polypeptide comprises the sequence of SEQ ID NO:171, (ii) the second heavy chain polypeptide comprises the sequence of SEQ ID NO:179, and (iii) the two light chain polypeptides each comprises the sequence of SEQ ID NO:26; or
(m) (i) the first heavy chain polypeptide comprises the sequence of SEQ ID NO:170, (ii) the second heavy chain polypeptide comprises the sequence of SEQ ID NO:179, and (iii) the two light chain polypeptides each comprises the sequence of SEQ ID NO:26; or
(n) (i) the first heavy chain polypeptide comprises the sequence of SEQ ID NO:170, (ii) the second heavy chain polypeptide comprises the sequence of SEQ ID NO:180, and (iii) the two light chain polypeptides each comprises the sequence of SEQ ID NO:26; or
(o) (i) the first heavy chain polypeptide comprises the sequence of SEQ ID NO:172, (ii) the second heavy chain polypeptide comprises the sequence of SEQ ID NO:179, and (iii) the two light chain polypeptides each comprises the sequence of SEQ ID NO:26; or
(p) (i) the first heavy chain polypeptide comprises the sequence of SEQ ID NO:169, (ii) the second heavy chain polypeptide comprises the sequence of SEQ ID NO:181, and (iii) the two light chain polypeptides each comprises the sequence of SEQ ID NO:26; or
(q) (i) the first heavy chain polypeptide comprises the sequence of SEQ ID NO:169, (ii) the second heavy chain polypeptide comprises the sequence of SEQ ID NO:182, and (iii) the two light chain polypeptides each comprises the sequence of SEQ ID NO:26; or
(r) (i) the first heavy chain polypeptide comprises the sequence of SEQ ID NO:171, (ii) the second heavy chain polypeptide comprises the sequence of SEQ ID NO:181, and (iii) the two light chain polypeptides each comprises the sequence of SEQ ID NO:26; or
(s) (i) the first heavy chain polypeptide comprises the sequence of SEQ ID NO:170, (ii) the second heavy chain polypeptide comprises the sequence of SEQ ID NO:181, and (iii) the two light chain polypeptides each comprises the sequence of SEQ ID NO:26; or
(t) (i) the first heavy chain polypeptide comprises the sequence of SEQ ID NO:170, (ii) the second heavy chain polypeptide comprises the sequence of SEQ ID NO:182, and (iii) the two light chain polypeptides each comprises the sequence of SEQ ID NO:26; or
(u) (i) the first heavy chain polypeptide comprises the sequence of SEQ ID NO:172, (ii) the second heavy chain polypeptide comprises the sequence of SEQ ID NO:181, and (iii) the two light chain polypeptides each comprises the sequence of SEQ ID NO:26.

70. A pharmaceutical composition comprising the protein of claim 1 and a pharmaceutically acceptable carrier.

71. An isolated polynucleotide comprising a nucleotide sequence encoding the protein of claim 1.

72. A vector comprising the polynucleotide of claim 71.

73. A host cell comprising the polynucleotide of claim 71 or the vector of claim 72.

74. A method for treating a cancer or treating brain metastasis of a cancer in a subject, the method comprising administering to the subject a therapeutically effective amount of the protein of claim 1 or the pharmaceutical composition of claim 70.

75. The method of claim 74, wherein the protein is adminstered in combination with a chemotherapy or radiation therapy.

76. The method of claim 74, wherein the cancer is a metastatic cancer.

77. The method of claim 74, wherein the cancer is a breast cancer.

78. The method of claim 74, wherein the cancer is a HER2-positive cancer.


SEQUENCE LISTING (196 sequences)

SEQ ID NO: 1 - protein, 449 aa, synthetic construct
  REGION 1..449: note = Description of sequence: Anti-HER2DII Fab-CH3C.35.23.4 with knob LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 2 - protein, 449 aa, synthetic construct
  REGION 1..449: note = Description of sequence: Anti-HER2DII Fab-CH3C.35.23.4 with hole LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 3 - protein, 449 aa, synthetic construct
  REGION 1..449: note = Description of sequence: Anti-HER2DII Fab-Fc with knob LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 449 aa, synthetic construct
  REGION 1..449: note = Description of sequence: Anti-HER2DII Fab-Fc with hole LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 449 aa, synthetic construct
  REGION 1..449: note = Description of sequence: Anti-HER2DII Fab-Fc with hole
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 450 aa, synthetic construct
  REGION 1..450: note = Description of sequence: Anti-HER2DIV Fab-CH3C.35.23.4 with knob LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 7 - protein, 450 aa, synthetic construct
  REGION 1..450: note = Description of sequence: Anti-HER2DIV Fab-CH3C.35.23.4 with hole LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 8 - protein, 450 aa, synthetic construct
  REGION 1..450: note = Description of sequence: Anti-HER2DIV Fab-Fc with knob LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 450 aa, synthetic construct
  REGION 1..450: note = Description of sequence: Anti-HER2DIV Fab-Fc with hole LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 450 aa, synthetic construct
  REGION 1..450: note = Description of sequence: Anti-HER2DIV Fab-Fc with hole
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 483 aa, synthetic construct
  REGION 1..483: note = Description of sequence: Anti-HER2DII scFv-CH3C.35.23.4 with knob LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 12 - protein, 483 aa, synthetic construct
  REGION 1..483: note = Description of sequence: Anti-HER2DII scFv-CH3C.35.23.4 with knob LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 13 - protein, 488 aa, synthetic construct
  REGION 1..488: note = Description of sequence: Anti-HER2DII scFv-CH3C.35.23.4 with hole LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 14 - protein, 488 aa, synthetic construct
  REGION 1..488: note = Description of sequence: Anti-HER2DII scFv-Fc with knob LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 483 aa, synthetic construct
  REGION 1..483: note = Description of sequence: Anti-HER2DII scFv-Fc with hole LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 483 aa, synthetic construct
  REGION 1..483: note = Description of sequence: Anti-HER2DII scFv-Fc with hole
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 483 aa, synthetic construct
  REGION 1..483: note = Description of sequence: Anti-HER2DII scFv-Fc with hole LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 484 aa, synthetic construct
  REGION 1..484: note = Description of sequence: Anti-HER2DIV scFv-CH3C.35.23.4 with knob LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 19 - protein, 489 aa, synthetic construct
  REGION 1..489: note = Description of sequence: Anti-HER2DIV scFv-CH3C.35.23.4 with hole LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 20 - protein, 489 aa, synthetic construct
  REGION 1..489: note = Description of sequence: Anti-HER2DIV scFv-Fc with knob LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 484 aa, synthetic construct
  REGION 1..484: note = Description of sequence: Anti-HER2DIV scFv-Fc with hole LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 484 aa, synthetic construct
  REGION 1..484: note = Description of sequence: Anti-HER2DIV scFv-Fc with hole (VL-VH scFv)_v1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 484 aa, synthetic construct
  REGION 1..484: note = Description of sequence: Anti-HER2DIV scFv-Fc with hole (VL-VH scFv) (with two Cys mutations in scFv and one Cys mutation in hinge)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 484 aa, synthetic construct
  REGION 1..484: note = Description of sequence: Anti-HER2DIV scFv-CH3C.35.23.4 with knob LALA (VL-VH scFv) (with two Cys mutations in scFv and one Cys mutation in hinge)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 25 - protein, 214 aa, synthetic construct
  REGION 1..214: note = Description of sequence: LC Anti-HER2DII Fab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, synthetic construct
  REGION 1..214: note = Description of sequence: LC Anti-HER2DIV Fab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 706 aa, synthetic construct
  REGION 1..706: note = Description of sequence: Anti-HER2DII Fab-CH3C.35.23.4 with knob LALA-anti-HER2DIV scFv (VL-VH scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 28 - protein, 706 aa, synthetic construct
  REGION 1..706: note = Description of sequence: Anti-HER2DII Fab-CH3C.35.23.4 with knob LALA-anti-HER2DIV scFv (VH-VL scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGGSGGGSGGGSGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 29 - protein, 706 aa, synthetic construct
  REGION 1..706: note = Description of sequence: Anti-HER2DII Fab-CH3C.35.23.4 with hole LALA-anti-HER2DIV scFv
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 30 - protein, 706 aa, synthetic construct
  REGION 1..706: note = Description of sequence: Anti-HER2DII Fab-Fc with knob LALA-anti-HER2DIV scFv
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 31 - protein, 705 aa, synthetic construct
  REGION 1..705: note = Description of sequence: Anti-HER2DII Fab-Fc with hole LALA-anti-HER2DIV scFv (VL-VH scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 32 - protein, 705 aa, synthetic construct
  REGION 1..705: note = Description of sequence: Anti-HER2DII Fab-Fc with hole-anti-HER2DIV scFv (VL-VH scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 33 - protein, 707 aa, synthetic construct
  REGION 1..707: note = Description of sequence: Anti-HER2DIV Fab-CH3C.35.23.4 with knob LALA-anti-HER2DII scFv (VL-VH scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 34 - protein, 707 aa, synthetic construct
  REGION 1..707: note = Description of sequence: Anti-HER2DIV Fab-CH3C.35.23.4 with knob LALA-anti-HER2DII scFv (VH-VL scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 35 - protein, 707 aa, synthetic construct
  REGION 1..707: note = Description of sequence: Anti-HER2DIV Fab-CH3C.35.23.4 with hole LALA-anti-HER2DII scFv (VL-VH scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 36 - protein, 707 aa, synthetic construct
  REGION 1..707: note = Description of sequence: Anti-HER2DIV Fab-Fc with knob LALA-anti-HER2DII scFv (VL-VH scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 37 - protein, 706 aa, synthetic construct
  REGION 1..706: note = Description of sequence: Anti-HER2DIV Fab-Fc with hole LALA-anti-HER2DII scFv (VL-VH scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 38 - protein, 706 aa, synthetic construct
  REGION 1..706: note = Description of sequence: Anti-HER2DIV Fab-Fc with hole-anti-HER2DII scFv (VL-VH scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 39 - protein, 706 aa, synthetic construct
  REGION 1..706: note = Description of sequence: Anti-HER2DIV Fab-Fc with hole LALA-anti-HER2DII scFv (VH-VL scFv)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 40 - protein, 706 aa, synthetic construct
  REGION 1..706: note = Description of sequence: Anti-HER2DIV scFv-anti-HER2DII Fab-CH3C.35.23.4 with knob LALA (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 41 - protein, 706 aa, synthetic construct
  REGION 1..706: note = Description of sequence: Anti-HER2DIV scFv-anti-HER2DII Fab-CH3C.35.23.4 with hole LALA (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 42 - protein, 706 aa, synthetic construct
  REGION 1..706: note = Description of sequence: Anti-HER2DIV scFv-anti-HER2DII Fab-Fc with knob LALA (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 706 aa, synthetic construct
  REGION 1..706: note = Description of sequence: Anti-HER2DIV scFv-anti-HER2DII Fab-Fc with hole LALA (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 706 aa, synthetic construct
  REGION 1..706: note = Description of sequence: Anti-HER2DIV scFv-anti-HER2DII Fab-Fc with hole (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 706 aa, synthetic construct
  REGION 1..706: note = Description of sequence: Anti-HER2DII scFv-anti-HER2DIV Fab-CH3C.35.23.4 with knob LALA (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 46 - protein, 706 aa, synthetic construct
  REGION 1..706: note = Description of sequence: Anti-HER2DII scFv-anti-HER2DIV Fab-CH3C.35.23.4 with knob LALA (VH-VL scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 47 - protein, 450 aa, synthetic construct; identical to SEQ ID NO: 7
  REGION 1..450: note = Description of sequence: Anti-HER2DII scFv-anti-HER2DIV Fab-CH3C.35.23.4 with hole LALA

SEQ ID NO: 48 - protein, 706 aa, synthetic construct
  REGION 1..706: note = Description of sequence: Anti-HER2DII scFv-anti-HER2DIV Fab-Fc with knob LALA (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 706 aa, synthetic construct
  REGION 1..706: note = Description of sequence: Anti-HER2DII scFv-anti-HER2DIV Fab-Fc with hole LALA (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 706 aa, synthetic construct
  REGION 1..706: note = Description of sequence: Anti-HER2DII scFv-anti-HER2DIV Fab-Fc with hole (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 706 aa, synthetic construct
  REGION 1..706: note = Description of sequence: Anti-HER2DII scFv-anti-HER2DIV Fab-Fc with hole LALA (VH-VL scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 472 aa, synthetic construct
  REGION 1..472: note = Description of sequence: LC anti-HER2DII Fab-anti-HER2DIV scFv (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 53 - protein, 470 aa, synthetic construct
  REGION 1..470: note = Description of sequence: LC anti-HER2DIV Fab-anti-HER2DII scFv (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 54 - protein, 470 aa, synthetic construct
  REGION 1..470: note = Description of sequence: LC anti-HER2DIV Fab-anti-HER2DII scFv (VH-VL scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 55 - protein, 471 aa, synthetic construct
  REGION 1..471: note = Description of sequence: Anti-HER2DIV scFv-LC anti-HER2DII Fab (VL-VH scFv)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 470 aa, synthetic construct
  REGION 1..470: note = Description of sequence: Anti-HER2DII scFv-LC anti-HER2DIV Fab (VL-VH scFv)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGGSGGGSGGGSGGGSGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 470 aa, synthetic construct
  REGION 1..470: note = Description of sequence: Anti-HER2DII scFv-LC anti-HER2DIV Fab (VH-VL scFv)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 578 aa, synthetic construct
  REGION 1..578: note = Description of sequence: Anti-HER2DIV VH-anti-HER2DII Fab-CH3C.35.23.4 with knob LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 59 - protein, 578 aa, synthetic construct
  REGION 1..578: note = Description of sequence: Anti-HER2DIV VH-anti-HER2DII Fab-CH3C.35.23.4 with hole LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 60 - protein, 578 aa, synthetic construct
  REGION 1..578: note = Description of sequence: Anti-HER2DIV VH-anti-HER2DII Fab-Fc with knob LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 578 aa, synthetic construct
  REGION 1..578: note = Description of sequence: Anti-HER2DIV VH-anti-HER2DII Fab-Fc with hole LALA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 578 aa, synthetic construct
  REGION 1..578: note = Description of sequence: Anti-HER2DIV VH-anti-HER2DII Fab-Fc with hole
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 578 aa, synthetic construct
  REGION 1..578: note = Description of sequence: Anti-HER2DII VH-anti-HER2DIV Fab-CH3C.35.23.4 with knob LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 64 - protein, 578 aa, synthetic construct
  REGION 1..578: note = Description of sequence: Anti-HER2DII VH-anti-HER2DIV Fab-CH3C.35.23.4 with hole LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 65 - protein, 578 aa, synthetic construct
  REGION 1..578: note = Description of sequence: Anti-HER2DII VH-anti-HER2DIV Fab-Fc with knob LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 578 aa, synthetic construct
  REGION 1..578: note = Description of sequence: Anti-HER2DII VH-anti-HER2DIV Fab-Fc with hole LALA
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - protein, 578 aa, synthetic construct
  REGION 1..578: note = Description of sequence: Anti-HER2DII VH-anti-HER2DIV Fab-Fc with hole
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 565 aa, synthetic construct
  REGION 1..565: note = Description of sequence: Anti-HER2DIV VL-anti-HER2DII Fab-CH3C.35.23.4 with knob LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 69 - protein, 565 aa, synthetic construct
  REGION 1..565: note = Description of sequence: Anti-HER2DIV VL-anti-HER2DII Fab-CH3C.35.23.4 with hole LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 70 - protein, 565 aa, synthetic construct
  REGION 1..565: note = Description of sequence: Anti-HER2DIV VL-anti-HER2DII Fab-Fc with knob LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 565 aa, synthetic construct
  REGION 1..565: note = Description of sequence: Anti-HER2DIV VL-anti-HER2DII Fab-Fc with hole LALA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 565 aa, synthetic construct
  REGION 1..565: note = Description of sequence: Anti-HER2DIV VL-anti-HER2DII Fab-Fc with hole
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 566 aa, synthetic construct
  REGION 1..566: note = Description of sequence: Anti-HER2DII VL-anti-HER2DIV Fab-CH3C.35.23.4 with knob LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 74 - protein, 566 aa, synthetic construct
  REGION 1..566: note = Description of sequence: Anti-HER2DII VL-anti-HER2DIV Fab-CH3C.35.23.4 with hole LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 75 - protein, 566 aa, synthetic construct
  REGION 1..566: note = Description of sequence: Anti-HER2DII VL-anti-HER2DIV Fab-Fc with knob LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 566 aa, synthetic construct
  REGION 1..566: note = Description of sequence: Anti-HER2DII VL-anti-HER2DIV Fab-Fc with hole LALA
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 566 aa, synthetic construct
  REGION 1..566: note = Description of sequence: Anti-HER2DII VL-anti-HER2DIV Fab-Fc with hole
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKASTKGPSVFEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 331 aa, synthetic construct
  REGION 1..331: note = Description of sequence: Anti-HER2DIV VL-LC anti-HER2DII Fab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 331 aa, synthetic construct
  REGION 1..331: note = Description of sequence: Anti-HER2DII VL-LC anti-HER2DIV Fab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 344 aa, synthetic construct
  REGION 1..344: note = Description of sequence: Anti-HER2DIV VH-LC anti-HER2DII Fab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRTVAAPSVFIDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 343 aa, synthetic construct
  REGION 1..343: note = Description of sequence: Anti-HER2DII VH-LC anti-HER2DIV Fab
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSRTVAAPSVFIDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 580 aa, synthetic construct
  REGION 1..580: note = Description of sequence: Anti-HER2DII Fab-CH3C.35.23.4 with knob LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 83 - protein, 586 aa, synthetic construct
  REGION 1..586: note = Description of sequence: Anti-HER2DII Fab-CH3C.35.23.4 with knob LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 84 - protein, 586 aa, synthetic construct
  REGION 1..586: note = Description of sequence: Anti-HER2DII Fab-CH3C.35.23.4 with hole LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGGSGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 85 - protein, 586 aa, synthetic construct
  REGION 1..586: note = Description of sequence: Anti-HER2DII Fab-Fc with knob LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGSGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 86 - protein, 579 aa, synthetic construct
  REGION 1..579: note = Description of sequence: Anti-HER2DII Fab-Fc with hole LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 87 - protein, 579 aa, synthetic construct
  REGION 1..579: note = Description of sequence: Anti-HER2DII Fab-Fc with hole-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 88 - protein, 585 aa, synthetic construct
  REGION 1..585: note = Description of sequence: Anti-HER2DII Fab-Fc with hole LALA-anti-HER2DIV VH
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 89 - protein, 580 aa, synthetic construct
  REGION 1..580: note = Description of sequence: Anti-HER2DIV Fab-CH3C.35.23.4 with knob LALA-anti-HER2DII VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 90 - protein, 585 aa, synthetic construct
  REGION 1..585: note = Description of sequence: Anti-HER2DIV Fab-CH3C.35.23.4 with knob LALA-anti-HER2DII VH
VQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 91 - protein, 586 aa, synthetic construct
  REGION 1..586: note = Description of sequence: Anti-HER2DIV Fab-CH3C.35.23.4 with hole LALA-anti-HER2DII VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGGSGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 92 - protein, 586 aa, synthetic construct
  REGION 1..586: note = Description of sequence: Anti-HER2DIV Fab-Fc with knob LALA-anti-HER2DII VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGSGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 93 - protein, 586 aa, synthetic construct
  REGION 1..586: note = Description of sequence: Anti-HER2DIV Fab-Fc with hole LALA-anti-HER2DII VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGSGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 94 - protein, 586 aa, synthetic construct
  REGION 1..586: note = Description of sequence: Anti-HER2DIV Fab-Fc with hole-anti-HER2DII VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGSGGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 95 - protein, 567 aa, synthetic construct
  REGION 1..567: note = Description of sequence: Anti-HER2DII Fab-CH3C.35.23.4 with knob LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 96 - protein, 573 aa, synthetic construct
  REGION 1..573: note = Description of sequence: Anti-HER2DII Fab-CH3C.35.23.4 with knob LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 97 - protein, 573 aa, synthetic construct
  REGION 1..573: note = Description of sequence: Anti-HER2DII Fab-CH3C.35.23.4 with hole LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGGSGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 98 - protein, 573 aa, synthetic construct
  REGION 1..573: note = Description of sequence: Anti-HER2DII Fab-Fc with knob LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGSGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 99 - protein, 566 aa, synthetic construct
  REGION 1..566: note = Description of sequence: Anti-HER2DII Fab-Fc with hole LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 100 - protein, 566 aa, synthetic construct
  REGION 1..566: note = Description of sequence: Anti-HER2DII Fab-Fc with hole-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 101 - protein, 572 aa, synthetic construct
  REGION 1..572: note = Description of sequence: Anti-HER2DII Fab-Fc with hole LALA-anti-HER2DIV VL
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 102 - protein, 568 aa, synthetic construct
  REGION 1..568: note = Description of sequence: Anti-HER2DIV Fab-CH3C.35.23.4 with knob LALA-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 103 - protein, 568 aa, synthetic construct
  REGION 1..568: note = Description of sequence: Anti-HER2DIV Fab-CH3C.35.23.4 with hole LALA-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 104 - protein, 568 aa, synthetic construct
  REGION 1..568: note = Description of sequence: Anti-HER2DIV Fab-Fc with knob LALA-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 105 - protein, 567 aa, synthetic construct
  REGION 1..567: note = Description of sequence: Anti-HER2DIV Fab-Fc with hole LALA-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 106 - protein, 567 aa, synthetic construct
  REGION 1..567: note = Description of sequence: Anti-HER2DIV Fab-Fc with hole-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 107 - protein, 573 aa, synthetic construct
  REGION 1..573: note = Description of sequence: Anti-HER2DIV Fab-Fc with hole LALA-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 108 - protein, 119 aa, synthetic construct [sequence omitted]

SEQ ID NO: 109 - protein, 120 aa, synthetic construct [sequence omitted]

SEQ ID NO: 110 - protein, 107 aa, synthetic construct [sequence omitted]

SEQ ID NO: 111 - protein, 107 aa, synthetic construct [sequence omitted]

SEQ ID NO: 112 - protein, 119 aa, synthetic construct
  REGION 1..119: note = Description of sequence: Anti-HER2DII VH region_Gly to Cys
EVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSS

SEQ ID NO: 113 - protein, 120 aa, synthetic construct
  REGION 1..120: note = Description of sequence: Anti-HER2DIV VH region_Gly to Cys
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 114 - protein, 107 aa, synthetic construct
  REGION 1..107: note = Description of sequence: Anti-HER2DII VL region_Gln to Cys
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIK

SEQ ID NO: 115 - protein, 107 aa, synthetic construct
  REGION 1..107: note = Description of sequence: Anti-HER2DIV VL region_Gln to Cys
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 116 - protein, 20 aa, synthetic construct
  REGION 1..20: note = Description of sequence: linker
GGSGGGSGGGSGGGSGGGSG

SEQ ID NO: 117 - protein, 5 aa, synthetic construct
  REGION 1..5: note = Description of sequence: linker
GGGGS

SEQ ID NO: 118 - protein, 10 aa, synthetic construct
  REGION 1..10: note = Description of sequence: linker
GGGGSGGGGS

SEQ ID NO: 119 - protein, 15 aa, synthetic construct
  REGION 1..15: note = Description of sequence: linker
GGGGSGGGGSGGGGS

SEQ ID NO: 120 - protein, 14 aa, synthetic construct
  REGION 1..14: note = Description of sequence: linker
GGGGSGGGGSGGGG

SEQ ID NO: 121 - protein, 12 aa, synthetic construct
  REGION 1..12: note = Description of sequence: linker
GGGGSGGGGSGG

SEQ ID NO: 122 - protein, 11 aa, synthetic construct
  REGION 1..11: note = Description of sequence: linker
GGGGGSGGGGS

SEQ ID NO: 123 - protein, 17 aa, synthetic construct
  REGION 1..17: note = Description of sequence: linker
GGGGGSGGGGGSGGGGS

SEQ ID NO: 124 - protein, 10 aa, synthetic construct
  REGION 1..10: note = Description of sequence: linker
GGGGSEPKSS

SEQ ID NO: 125 - protein, 9 aa, synthetic construct
  REGION 1..9: note = Description of sequence: linker
ASTKGPSVF

SEQ ID NO: 126 - protein, 10 aa, synthetic construct
  REGION 1..10: note = Description of sequence: linker
RTVAAPSVFI

SEQ ID NO: 127 - protein, 15 aa, synthetic construct
  REGION 1..15: note = Description of sequence: human IgG1 hinge region
EPKSCDKTHTCPPCP

SEQ ID NO: 128 - protein, 10 aa, synthetic construct
  REGION 1..10: note = Description of sequence: Partial hinge region
DKTHTCPPCP

SEQ ID NO: 129 - protein, 15 aa, synthetic construct
  REGION 1..15: note = Description of sequence: Hinge region with Cys to Ser mutation
EPKSSDKTHTCPPCP

SEQ ID NO: 130 - protein, 217 aa, synthetic construct
  REGION 1..217: note = Description of sequence: Wild-type human Fc sequence positions 231-447 EU index numbering
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 217 aa, synthetic construct
  REGION 1..217: note = Description of sequence: Fc sequence with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 217 aa, synthetic construct
  REGION 1..217: note = Description of sequence: Fc sequence with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 217 aa, synthetic construct
  REGION 1..217: note = Description of sequence: Fc sequence with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 134 - protein, 217 aa, synthetic construct
  REGION 1..217: note = Description of sequence: Fc sequence with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - protein, 217 aa, synthetic construct
  REGION 1..217: note = Description of sequence: Clone CH3C.35.23.4
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 136 - protein, 217 aa, synthetic construct
  REGION 1..217: note = Description of sequence: Clone CH3C.35.23.4 with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 137 - protein, 217 aa, synthetic construct
  REGION 1..217: note = Description of sequence: Clone CH3C.35.23.4 with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 138 - protein, 217 aa, synthetic construct
  REGION 1..217: note = Description of sequence: Clone CH3C.35.23.4 with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 139 - protein, 217 aa, synthetic construct
  REGION 1..217: note = Description of sequence: Clone CH3C.35.23.4 with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 140 - protein, 217 aa, synthetic construct
  REGION 1..217: note = Description of sequence: Clone CH3C.35.23.2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 141 - protein, 217 aa, synthetic construct
  REGION 1..217: note = Description of sequence: Clone CH3C.35.23.2 with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 142 - protein, 217 aa, synthetic construct
  REGION 1..217: note = Description of sequence: Clone CH3C.35.23.2 with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 143 - protein, 217 aa, synthetic construct
  REGION 1..217: note = Description of sequence: Clone CH3C.35.23.2 with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 144 - protein, 217 aa, synthetic construct
  REGION 1..217: note = Description of sequence: Clone CH3C.35.23.2 with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 145 - protein, 217 aa, synthetic construct
  REGION 1..217: note = Description of sequence: Clone CH3C.35.20.1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 146 - protein, 217 aa, synthetic construct
  REGION 1..217: note = Description of sequence: Clone CH3C.35.20.1 with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 147 - protein, 217 aa, synthetic construct
  REGION 1..217: note = Description of sequence: Clone CH3C.35.20.1 with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 148 - protein, 217 aa, synthetic construct
  REGION 1..217: note = Description of sequence: Clone CH3C.35.20.1 with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 149 - protein, 217 aa, synthetic construct
  REGION 1..217: note = Description of sequence: Clone CH3C.35.20.1 with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESFGTEWSSYKTTPPVLDSDGSFFLVSKLTVTKEEWQQGFVFSCSVMHEALHNHYTQKSLSLSPGK

SEQ ID NO: 150 - protein, 760 aa, synthetic construct
  REGION 1..760: note = Description of sequence: Human transferrin receptor protein 1 (TFR1)
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 151 - protein, 4 aa, synthetic construct
  REGION 1..4: note = Description of sequence: Linker
GGSG

SEQ ID NO: 152 - protein, 4 aa, synthetic construct
  REGION 1..4: note = Description of sequence: Linker
GSGG

SEQ ID NO: 153 - protein, 4 aa, synthetic construct
  REGION 1..4: note = Description of sequence: Linker
SGGG

SEQ ID NO: 154 - protein, 450 aa, synthetic construct
  REGION 1..450: note = Description of sequence: Anti-HER2DIV Fab-wild-type human Fc sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 449 aa, synthetic construct
  REGION 1..449: note = Description of sequence: Anti-HER2DII Fab-wild-type Fc sequence
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 706 aa, synthetic construct
  REGION 1..706: note = Description of sequence: Anti-HER2DII scFv-anti-HER2DIV Fab-Fc with hole_v1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 157 - protein, 706 aa, synthetic construct; identical to SEQ ID NO: 38
  REGION 1..706: note = Description of sequence: Anti-HER2DIV Fab-Fc with hole-anti-HER2DII scFv (VL-VH scFv)

SEQ ID NO: 158 - protein, 573 aa, synthetic construct
  REGION 1..573: note = Description of sequence: Anti-HER2DIV Fab-Fc with hole-anti-HER2DII VL
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 159 - protein, 479 aa, synthetic construct
  REGION 1..479: note = Description of sequence: Anti-HER2DIV scFv-Fc with hole (VL-VH scFv)_v2 3(G4S)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 160 - protein, 701 aa, synthetic construct
  REGION 1..701: note = Description of sequence: Anti-HER2DII scFv-anti-HER2DIV Fab-Fc with hole_v2 5(GGSG)_(G4S)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 161 - protein, 696 aa, synthetic construct
  REGION 1..696: note = Description of sequence: Anti-HER2DII scFv-anti-HER2DIV Fab-Fc with hole_v3
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 162 - protein, 701 aa, synthetic construct
  REGION 1..701: note = Description of sequence: Anti-HER2DII scFv-anti-HER2DIV Fab-Fc with hole_v4
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 163 - protein, 449 aa, synthetic construct
  REGION 1..449: note = Description of sequence: Anti-HER2DII Fab-CH3C.35.23.4 with knob LALA.kD
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 164 - protein, 449 aa, synthetic construct
  REGION 1..449: note = Description of sequence: Anti-HER2DII Fab-CH3C.35.23.4 with knob LALA.kD.PG
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 165 - protein, 449 aa, synthetic construct
  REGION 1..449: note = Description of sequence: Anti-HER2DII Fab-CH3C.35.23.4 with knob LALA kD.PS
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 166 - protein, 449 aa, synthetic construct
  REGION 1..449: note = Description of sequence: Anti-HER2DII Fab-CH3C.35.23.4 with knob LALA.PG
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 167 - protein, 449 aa, synthetic construct
  REGION 1..449: note = Description of sequence: Anti-HER2DII Fab-CH3C.35.23.4 with knob LALA.PS
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 168 - protein, 450 aa, synthetic construct
  REGION 1..450: note = Description of sequence: Anti-HER2DIV Fab-CH3C.35.23.4 with knob LALA.kD
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 169 - protein, 450 aa, synthetic construct
  REGION 1..450: note = Description of sequence: Anti-HER2DIV Fab-CH3C.35.23.4 with knob LALA.kD.PG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 170 - protein, 450 aa, synthetic construct
  REGION 1..450: note = Description of sequence: Anti-HER2DIV Fab-CH3C.35.23.4 with knob LALA.kD.PS
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 171 - protein, 450 aa, synthetic construct
  REGION 1..450: note = Description of sequence: Anti-HER2DIV Fab-CH3C.35.23.4 with knob LALA.PG
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 172 - protein, 450 aa, synthetic construct
  REGION 1..450: note = Description of sequence: Anti-HER2DIV Fab-CH3C.35.23.4 with knob LALA.PS
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 173 - protein, 484 aa, synthetic construct
  REGION 1..484: note = Description of sequence: Anti-HER2DIV scFv-Fc with hole
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 174 - protein, 484 aa, synthetic construct
  REGION 1..484: note = Description of sequence: Anti-HER2DIV scFv-Fc with hole (VL-VH scFv)_v1_hDE
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 175 - protein, 479 aa, synthetic construct
  REGION 1..479: note = Description of sequence: Anti-HER2DIV scFv-Fc with hole (VL-VH scFv)_v2 3(G4S)_hE
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 176 - protein, 479 aa, synthetic construct
  REGION 1..479: note = Description of sequence: Anti-HER2DIV scFv-Fc with hole (VL-VH scFv)_v2 3(G4S)_hDE
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 177 - protein, 706 aa, synthetic construct
  REGION 1..706: note = Description of sequence: Anti-HER2DII scFv-anti-HER2DIV Fab-Fc with hole_v1_hE
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 178 - protein, 706 aa, synthetic construct
  REGION 1..706: note = Description of sequence: Anti-HER2DII scFv-anti-HER2DIV Fab-Fc with hole_v1_hDE
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 179 - protein, 701 aa, synthetic construct
  REGION 1..701: note = Description of sequence: Anti-HER2DII scFv-anti-HER2DIV Fab-Fc with hole_v2 5(GGSG)_(G4S)_hE
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 180 - protein, 701 aa, synthetic construct
  REGION 1..701: note = Description of sequence: Anti-HER2DII scFv-anti-HER2DIV Fab-Fc with hole_v2 5(GGSG)_(G4S)_hDE
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGSGGGSGGGSGGGSGGGSGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 181 - protein, 701 aa, synthetic construct
  REGION 1..701: note = Description of sequence: Anti-HER2DII scFv-anti-HER2DIV Fab-Fc with hole_v4
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 182 - protein, 701 aa, synthetic construct
  REGION 1..701: note = Description of sequence: Anti-HER2DII scFv-anti-HER2DIV Fab-Fc with hole_v4 3(G4S)_2(G4S)_hDE
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 183 - protein, 217 aa, synthetic construct
  REGION 1..217: note = Description of sequence: Fc sequence with hole mutations.hD
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 184 - protein, 217 aa, synthetic construct
  REGION 1..217: note = Description of sequence: Fc sequence with hole mutations.hE
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 185 - protein, 217 aa, synthetic construct
  REGION 1..217: note = Description of sequence: Fc sequence with hole mutations.hDE
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 186 - protein, 217 aa, synthetic construct
  REGION 1..217: note = Description of sequence: Clone CH3C.35.23.4 with knob& LALA& and kD
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 187 - protein, 217 aa, synthetic construct
  REGION 1..217: note = Description of sequence: Clone CH3C.35.23.4 with knob& LALA& and kE
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 188 - protein, 217 aa, synthetic construct
  REGION 1..217: note = Description of sequence: Clone CH3C.35.23.4 with knob& LALA& and kDE
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 189 - protein, 217 aa, synthetic construct
  REGION 1..217: note = Description of sequence: Clone CH3C.35.23.4 with knob& LALA& and PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 190 - protein, 217 aa, synthetic construct
  REGION 1..217: note = Description of sequence: Clone CH3C.35.23.4 with knob& LALA& and PS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 191 - protein, 217 aa, synthetic construct
  REGION 1..217: note = Description of sequence: Clone CH3C.35.23.4 with knob& LALA& PG& and kD
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 192 - protein, 217 aa, synthetic construct
  REGION 1..217: note = Description of sequence: Clone CH3C.35.23.4 with knob& LALA& PS& and kD
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 193 - protein, 217 aa, synthetic construct
  REGION 1..217: note = Description of sequence: Clone CH3C.35.23.4 with knob& LALA& PG& and kE
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 194 - protein, 217 aa, synthetic construct
  REGION 1..217: note = Description of sequence: Clone CH3C.35.23.4 with knob& LALA& PS& and kE
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 195 - protein, 217 aa, synthetic construct
  REGION 1..217: note = Description of sequence: Clone CH3C.35.23.4 with knob& LALA& PG& and kDE
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 196 - protein, 217 aa, synthetic construct
  REGION 1..217: note = Description of sequence: Clone CH3C.35.23.4 with knob& LALA& PS& and kDE
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK